SEQUENCE LISTING (33 sequences)

SEQ ID NO: 1 - DNA, 4099 bp, Homo sapiens
tttgcagagtactagacggtgaggagtgaggtggggaggaagaaccccaaatttcttgccctatttgcccccatcaaattcctcaacatggtcaacattgtttctagaacatgtcctgggattgtgggaagggagaccactcatttgcccctccctaaagcttctgggcttccagacccagctactttgcggaactcagcaacccaggcatctctgagtctccgcccaagaccgggatgccccccaggggaggtgtccgggagcccagcctttcccagatagcacgctccgccagtcccaagggtgcgcaaccggctgcactcccctcccgcgacccagggcccgggagcagcccccatgacccacacgcacgtctgcagcagccccgctcacgccccggcgagcctcaacccaggcgtcctgcccctgctctgaccccgggtggcccctacccctggcgacccctcacgcacacagcctctcccccacccccacccgcgcacgcacacatgcagataacagccccgacccccggccagagccgcagagtccctgggccaccccggccgctcgctgcgctgcgccgcaccgcgctgtcctcccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacggtgagtactcgcgggctgggcgctcccgcccgcccgggtccctgtttgagcggggatttagcgccccggctattggccaggaggtggctgggttcaaggaccggcgacttgtcaaggaccccggaagggggaggggggtggggcagcctccacgtgccagcggggacttgggggagtccttggggatggcaaaaacctgacctgtgaaggggacacagtttgggggttgaggggaagaaggtttgggggttctgctgtgccagtggagaggaagctgataagctgataacctgggcgctggagccaccacttatctgccagaggggaagcctctgtcacaccaggattgaagtttggccggagaagtggatgctggtagctgggggtggggtgtgcacacggcagcaggattgaatgaaggccagggaggcagcacctgagtgcttgcatggttggggacaggaaggacgagctggggcagagacgtggggatgaaggaagctgtccttccacagccacccttctccctccccgcctgactctcagcctggctatctgttctagaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtgagaccccttccccagcacattccacagaactcacgctcagggcttcagggaactcctcccagatccaggaacctggcacttggtttggggtggagttgggaagctagacactgcccccctacataagaataagtctggtggccccaaaccatacctggaaactaggcaaggagcaaagccagcagatcctacggcctgtgggccagggccagagccttcagggacccttgactccccgggctgtgtgcatttcagacgggctgtgctgaacactgcagcttgaatgagaatatcactgtcccagacaccaaagttaatttctatgcctggaagaggatggaggtgagttcctttttttttttttttcctttcttttggagaatctcatttgcgagcctgattttggatgaaagggagaatgatcgagggaaaggtaaaatggagcagcagagatgaggctgcctgggcgcagaggctcacgtctataatcccaggctgagatggccgagatgggagaattgcttgagccctggagtttcagaccaacctaggcagcatagtgagatcccccatctctacaaacatttaaaaaaattagtcaggtgaggtggtgcatggtggtagtcccagatatttggaaggctgaggcgggaggatcgcttgagcccaggaatttgaggctgcagtgagctgtgatcacaccactgcactccagcctcagtgacagagtgaggccctgtctcaaaaaagaaaagaaaaaagaaaaataatgagggctgtatggaatacattcattattcattcactcactcactcactcactcattcattcattcattcattcaacaagtcttattgcataccttctgtttgctcagcttggtgcttggggctgctgaggggcaggagggagagggtgacatgggtcagctgactcccagagtccactccctgtaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgtaagaaggggagaagggtcttgctaaggagtacaggaactgtccgtattccttccctttctgtggcactgcagcgacctcctgttttctccttggcagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggataacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaatatgactcttggcttttctgttttctgggaacctccaaatcccctggctctgtcccactcctggcagcagtgcagcaggtccaggtccgggaaacgaggggtggagggggctgggccctacgtgctgtctcacacagcctgtctgacctctcgaccctaccgggcctgaggccacaagctctgcctacgctggtcaataaggtggctccattcaaggcctcaccgcagtaaggcagctgccaaccctgcccagggcaaggctgcagtgcgctgagattgtcatcaaggagagggaggccagaggacgggtccttttgggagttttgggggctggtaacagctgccaaccctggtcactggcccttgttaatttctgcctcttctttggtctcttcggctgatgccacactcccagcatcagccttccgcctcaatgcacatcctcgctaagtccagctgcctcctttactcacctggggactgtcaccatgaatactcgcctctgaaattcacaaaaggggcggtacacacaggtgcggttggagccacctctataaacagcatctgctcggggacaagggccactctaggtggtcca

SEQ ID NO: 2 - DNA, 1328 bp, Homo sapiens
cccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggataacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccacc

SEQ ID NO: 3 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 4 - protein, 164 aa, Artificial
  EPOv1 protein
MGVHECPAWLWLLLSLLSLPLGLPVLGAPPRLICDSRVLERYLLEAKEAENITVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 5 - DNA, 492 bp, Artificial
  EPOv1 cDNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 6 - protein, 104 aa, Artificial
  EPOv2 protein
MGVHECPAWLWLLLSLLSLPLGLPVLGAPPRLICDSRVLERYLLEAKEAENITKEAISPPDAASAAPLRTITADTFRKLFRVYSNFLRGKLKLYTGEACRTGDR

SEQ ID NO: 7 - DNA, 312 bp, Artificial
  EPOv2 cDNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 8 - protein, 12 aa, Artificial
  EPOv3 C-ter
VSRSGHFCLPFL

SEQ ID NO: 9 - protein, 154 aa, Artificial
  EPOv3 protein
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQVSRSGHFCLPFL

SEQ ID NO: 10 - DNA, 36 bp, Artificial
  EPOv3 cDNA C-Term
gtgagtaggagcggacacttctgcttgccctttctg

SEQ ID NO: 11 - DNA, 462 bp, Artificial
  EPOv3 cDNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctg

SEQ ID NO: 12 - DNA, 168 bp, Artificial
  EPOv cDNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctga

SEQ ID NO: 13 - protein, 55 aa, Artificial
  EPOv protein
MGVHECPAWLWLLLSLLSLPLGLPVLGAPPRLICDSRVLERYLLEAKEAENITTG

SEQ ID NO: 14 - DNA, 168 bp, Artificial
  EPOv C34S cDNA
atgggggtgcacgaatgtcctgcctggctgtggcttctcctgtccctgctgtcgctccctctgggcctcccagtcctgggcgccccaccacgcctcatcagtgacagccgagtcctggagaggtacctcttggaggccaaggaggccgagaatatcacgacgggctga

SEQ ID NO: 15 - protein, 55 aa, Artificial
  EPOv C34S
MGVHECPAWLWLLLSLLSLPLGLPVLGAPPRLISDSRVLERYLLEAKEAENITTG

SEQ ID NO: 16 - DNA, 25 bp, Artificial
  RecF1
tgagggaccccggccaggcgcggag

SEQ ID NO: 17 - DNA, 25 bp, Artificial
  RecR1
atgcccaggtggacacacctggtca

SEQ ID NO: 18 - DNA, 57 bp, Artificial
  CloF1
ggggacaagtttgtacaaaaaagcaggcttcgccaccatgggggtgcacgaatgtcc

SEQ ID NO: 19 - DNA, 71 bp, Artificial
  CloR1
ggggaccactttgtacaagaaagctgggtttcaatggtgatggtgatggtgtctgtcccctgtcctgcagg

SEQ ID NO: 20 - DNA, 7349 bp, Artificial
  pEAK12d_EPOv1-HIS
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttgggttcattctcaagcctcagacagtggttcaaattaatacgactcactatagggagacttctttctcccatttcaggtgtcgtaagctatcaaacaagtttgtacaaaaaagcaggcttcgccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagacaccatcaccatcaccattgaaacccagctttcttgtacaaagtggttcgatggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatccgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccggcacctcctccagccccagcacctccaccagccccagctcccccagctccagccccaccagcaccagcccctccagccccaccagccccagcccctccggcacctcctccagccccagcacctccaccagccccagctcccccagctccagccccaccagcaccagcccctccagccccaccagccccagcccctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaagctaagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 21 - DNA, 7169 bp, Artificial
  pEAK12d_EPOv2-HIS
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttgggttcattctcaagcctcagacagtggttcaaattaatacgactcactatagggagacttctttctcccatttcaggtgtcgtaagctatcaaacaagtttgtacaaaaaagcaggcttcgccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagacaccatcaccatcaccattgaaacccagctttcttgtacaaagtggttcgatggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatccgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccggcacctcctccagccccagcacctccaccagccccagctcccccagctccagccccaccagcaccagcccctccagccccaccagccccagcccctccggcacctcctccagccccagcacctccaccagccccagctcccccagctccagccccaccagcaccagcccctccagccccaccagccccagcccctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaagctaagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 22 - DNA, 71 bp, Artificial
  CloR3
ggggaccactttgtacaagaaagctgggtttcaatggtgatggtgatggtgcagaaagggcaagcagaagt

SEQ ID NO: 23 - DNA, 7319 bp, Artificial
  pEAK12d_EPOv3-HIS
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttgggttcattctcaagcctcagacagtggttcaaattaatacgactcactatagggagacttctttctcccatttcaggtgtcgtaagctatcaaacaagtttgtacaaaaaagcaggcttcgccaccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgcaccatcaccatcaccattgaaacccagctttcttgtacaaagtggttcgatggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatccgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccggcacctcctccagccccagcacctccaccagccccagctcccccagctccagccccaccagcaccagcccctccagccccaccagccccagcccctccggcacctcctccagccccagcacctccaccagccccagctcccccagctccagccccaccagcaccagcccctccagccccaccagccccagcccctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaagctaagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 24 - DNA, 20 bp, Artificial
  AS671
caagtgtatcatatgccaag

SEQ ID NO: 25 - DNA, 20 bp, Artificial
  AS672
caagcagcaagcatatgcag

SEQ ID NO: 26 - DNA, 35 bp, Artificial
  AS673
cacgacgggccaccatcaccatcaccattgaaacc

SEQ ID NO: 27 - DNA, 23 bp, Artificial
  AS674
ggtggcccgtcgtgatattctcg

SEQ ID NO: 28 - DNA, 25 bp, Artificial
  AS675
ccacgcctcatcagtgacagccgag

SEQ ID NO: 29 - DNA, 25 bp, Artificial
  AS676
ctcggctgtcactgatgaggcgtgg

SEQ ID NO: 30 - DNA, 17 bp, Artificial
  21M13
gtaaaacgacggccagt

SEQ ID NO: 31 - DNA, 18 bp, Artificial
  M13rev
caggaaacagctatgacc

SEQ ID NO: 32 - DNA, 25 bp, Artificial
  pDONR forward primer
tcgcgttaacgctagcatggatctc

SEQ ID NO: 33 - DNA, 24 bp, Artificial
  pDONR reverse primer
gtaacatcagagattttgagacac

(19) United States
(12) Patent Application Publication
(10) Pub. No.: US 2008/0260746 A1
(43) Pub. Date: Oct. 23, 2008

(54) Erythropoietin Polypeptides and Uses Thereof

(75) Inventors: Hadi Abderrahim, Maisons-Alfort (FR); Gwenael Primas, Auvers St Georges (FR); Yolande Chvatchko, Confignon (CH); Kinsey Maundrell, Chene-Bougeries (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Laboratoires Serono SA, Coinsins, Vaud (CH)

(21) Appl. No.: 12/094,869

(22) PCT Filed: Nov. 23, 2006
(86) PCT No.: PCT/EP06/68859

§ 371 Date: May 23, 2008

Related U.S. Application Data
(60) Provisional application No. 60/753,668, filed on Dec. 22, 2005.
(60) Provisional application No. 60/753,706, filed on Dec. 22, 2005.

(30) Foreign Application Priority Data
Nov. 24, 2005 (EP) 05111262.1
Nov. 24, 2005 (EP) 05111265.4

(51) Int. Cl. A61K 39/395; C07K 14/505; C07H 21/00; C12N 5/00; A61K 38/14; A61P 25/00; A61P 9/00; A61P 35/00; C12P 21/00; C12N 1/00; C07K 16/00

(52) U.S. Cl. 424/139.1; 530/397; 536/23.51; 435/325; 435/243; 530/387.9; 435/69.1; 514/8

(57) ABSTRACT

The present invention relates to EPO polypeptides and their uses, particularly for therapeutic or prophylactic treatment in human subjects. The invention also relates to nucleic acids encoding said polypeptides, vectors comprising such nucleic acids and recombinant cells containing the same. The invention further discloses methods of producing such polypeptides, as well as methods and tools for detecting or dosing these polypeptides in any sample.

The present invention relates to new EPO (Erythropoietin) polypeptides and their uses, particularly for therapeutic or prophylactic treatment in human subjects. The invention also relates to nucleic acids encoding said polypeptides, vectors comprising such nucleic acids and recombinant cells containing the same, as well as corresponding pharmaceutical compositions. The invention further discloses methods of producing such polypeptides, as well as methods and tools for detecting or dosing these polypeptides in any sample.

### BACKGROUND

Erythropoietin (EPO) is a hematopoietic growth hormone produced in the kidney and involved in stimulating production of red blood cells (erythrocytes) (Carnot, P and Deflandre, C (1906) C. R. Acad. Sci. 143: 432; Erslev, A J (1953) Blood 8: 349; Reissmann, K R (1950) Blood 5: 372; Jacobson, L O, Goldwasser, E, Freid, W and Plzak, L F (1957) Nature 179: 6331-4). EPO stimulates the division and differentiation of committed erythroid progenitors in the bone marrow and exerts its biological activity by binding to receptors on erythroid precursors (Krantz, B S (1991) Blood 77: 419). It activates cells by binding and orientating two cell-surface erythropoietin receptors (EPORs) which trigger an intracellular phosphorylation cascade (Damen J E, Krystal G. (1996) Exp Hematol. 24(13):1455-9).

Human erythropoietin is an acidic glycoprotein of approximately 34,000 dalton (34 kDa) molecular weight. Native human EPO may occur in three forms: alpha, beta and asialo. The alpha and beta forms differ slightly in carbohydrate components, but have the same potency, biological activity and molecular weight. The asialo form is an alpha or beta form with the terminal carbohydrate (sialic acid) removed.

EPO is normally present in very low concentration in plasma when the body is in a healthy state. This normal low concentration is enough to stimulate constant low-level replacement of red blood cells which are lost normally through cell aging. The amount of EPO in the circulation is increased under conditions of hypoxia when oxygen transport by blood cells in the circulation is reduced. Hypoxia may be caused by loss of large amounts of blood through haemorrhage, destruction of red blood cells by over-exposure to radiation, reduction in oxygen intake due to high altitudes or prolonged unconsciousness, or various forms of anemia. In response to tissues undergoing hypoxic stress, erythropoietin will increase red blood cell production by stimulating the conversion of primitive precursor cells in the bone marrow into proerythroblasts which subsequently mature, synthesize hemoglobin and are released into the circulation as red blood cells. When the number of red blood cells in circulation is greater than needed for normal tissue oxygen requirements, EPO in circulation decreases.

EPO has also been reported to be neuroprotective (Siren A L, et al., (Proc Natl Acad Sci USA. 2001, 98(7):4044-9) and cardioprotective (Parsa C J et al. (J Clin Invest. 2003. 112(7):999-1007).

Recombinant human EPO (rHuEPO) is currently being used to treat patients with anemias associated with chronic renal failure, AIDS patients with anemia due to treatment with zidovudine, nonmyeloid malignancies in patients treated with chemotherapeutic agents, perioperative surgical patients, and autologous blood donation.

Considering the biological activities of EPO, it would be highly valuable to obtain biologically active EPO variants. Ideally, such variants would include ligands, such as agonists, reverse agonists, partial agonists, mixed agonists/antagonists and full antagonists, which bind at the EPO receptor and initiate, inhibit, activate, or otherwise control, the biological activities of this protein. It would be of particular interest to obtain new agonists of human EPO.

It would also be of particular interest to obtain new biologically active EPO variants having a tissue protective activity (in particular neurotrophic activity) in a mammal in particular in human, without substantially increasing hematocrit level in said mammal.

### SUMMARY OF THE PRESENT INVENTION

The present invention relates to novel EPO polypeptides and their uses, particularly for therapeutic or prophylactic treatment in human subjects. The invention further discloses methods of producing such polypeptides, as well as methods and tools for detecting or dosing these polypeptides in a sample. The invention also discloses nucleic acids encoding said polypeptides, vectors comprising such nucleic acids, in particular expression vectors, and recombinant cells containing the same, as well as corresponding pharmaceutical compositions. Further included are antibodies specific for the novel EPO polypeptides of the present invention.

The present invention results in part from the identification, isolation and characterization of novel transcriptional variants of EPO having particular structural and biological properties. These transcriptional variants and derivatives thereof represent valuable pharmaceutical products.

The present invention results also in part from the characterization of novel transcriptional variants and shorter version of EPO having a tissue protective activity without substantially increasing hematocrit level. These shorter versions of EPO and transcriptional variants and derivatives thereof represent valuable pharmaceutical products. The present invention results also in part from the identification of the domain of EPO having tissue protective activity (in particular neurotrophic activity) but lacking an hematotrophic activity.

An object of this invention thus resides in an isolated erythropoietin variant polypeptide having a tissue protective activity in mammals in particular in human, without substantially increasing hematocrit level in said mammal.

Another object of this invention resides in an isolated erythropoietin variant polypeptide, said variant polypeptide consisting of a polypeptide differing from the sequence set forth at SEQ ID NO: 3 by the lack of at least one of the amino acids 56 (Cysteine) to 193 (Arginine) of SEQ ID NO: 3, or a variant or an analog of said polypeptide. In a particular embodiment, the invention resides in an isolated erythropoietin variant polypeptide, said variant polypeptide consisting of a polypeptide differing from the sequence set forth at SEQ ID NO: 3 by the lack of amino acids 56 (Cysteine) to 193 (Arginine) of SEQ ID NO: 3 or a variant or an analog of said polypeptide. The polypeptide lacking amino acids 56 to 193 of SEQ ID NO: 3 is shown at SEQ ID NO: 13 (named hereafter EPOv). In a preferred embodiment, these peptides are mature peptide lacking the N-terminal signal peptide.

Another object of this invention resides in a polypeptide comprising or consisting of an EPO polypeptide differing from the sequence set forth at SEQ ID NO: 3 by the lack of at least one of the amino acids 54 (Threonine) to 82 (Glutamic acid) of SEQ ID NO: 3 or a variant or an analog of said polypeptides. In a particular embodiment, the invention resides in an isolated polypeptide comprising or consisting of an EPO polypeptide differing from the sequence set forth at SEQ ID NO: 3 by the lack of the amino acids 54 (Threonine) to 82 (Glutamic acid) of SEQ ID NO: 3. The polypeptide lacking amino acids 54 to 82 of SEQ ID NO: 3 is shown at SEQ ID NO: 4 (named hereafter EPOvl) and is a novel transcriptional variant of EPO, which is encoded by exons 1, 2, 4 and 5 of the human gene EPO (the transcript of this transcriptional variant therefore lacks the internal exon 3). In a preferred embodiment, these peptides are mature peptide lacking the N-terminal signal peptide.

Another object of this invention resides in an isolated polypeptide comprising or consisting of an EPO polypeptide differing from the sequence set forth at SEQ ID NO: 3 by the lack of at least one of the amino acids 54 (Threonine) to 142 (Glutamine) of SEQ ID NO: 3 or a variant or an analog of said polypeptides. In a particular embodiment, the invention resides in an isolated polypeptide comprising or consisting of an EPO polypeptide differing from the sequence set forth at SEQ ID NO: 3 by the lack of the amino acids 54 (Threonine) to 142 (Glutamine) of SEQ ID NO: 3. The polypeptide lacking amino acids 54 to 142 of SEQ ID NO: 3 is shown at SEQ ID NO: 6 (named hereafter EPOv2) and is a novel transcriptional variant of EPO, which is encoded by exons 1, 2 and 5 of the human gene EPO (the transcript of this transcriptional variant therefore lacks the internal exons 3 and 4). In a preferred embodiment, these peptides are mature peptide lacking the N-terminal signal peptide.

Another object of the present invention resides in an isolated polypeptide comprising or consisting of the sequence set forth at SEQ ID NO: 8 or a variant of the polypeptide set forth at SEQ ID NO: 8. The polypeptide having the sequence set forth at SEQ ID NO: 8 corresponds to the C-terminal part of a novel transcriptional variant of EPO disclosed here for the first time and is encoded by the 3′ end of exon 4A. Said exon 4A is longer at the 3′ end as compared to exon 4 which encode the wild-type EPO (see FIGS. 3 and 6).

In a further aspect, the invention resides in an isolated polypeptide comprising or consisting of the sequence set forth at SEQ ID NO: 9 or a variant of said polypeptide. The polypeptide of SEQ ID NO: 9 (named hereafter EPOv3) is a novel transcriptional variant of EPO, which is encoded by exons 1, 2, 3 and 4A of the human gene EPO. In a preferred embodiment, these peptides are mature peptide lacking the N-terminal signal peptide.

Another object of the present invention resides in a fusion protein comprising an EPO polypeptide or variant or analog as defined above operably linked to an additional amino acid domain.

A further object of this invention resides in a nucleic acid encoding an EPO polypeptide or variant or analog or a fusion protein as defined above, as well as any cloning or expression vector comprising such a nucleic acid.

The invention also relates to recombinant host cells comprising a vector or nucleic acid as defined above, as well as to methods of producing an EPO polypeptide or variant or analog as defined above using such recombinant cells.

Another object of the present invention resides in a polypeptide as defined above in the form of active conjugates or complex.

A further object of this invention also relates to an antibody, or a fragment or derivative of such an antibody, which selectively binds to a polypeptide as defined above.

The invention also relates to an immunoconjugate comprising an antibody as defined above conjugated to a heterologous moiety.

A further object of this invention also resides in a pharmaceutical composition comprising a polypeptide, nucleic acid, vector or recombinant cell as defined above and a pharmaceutically acceptable carrier, excipient, or stabilizer.

The invention further relates to a method of treating, preventing or ameliorating the symptoms of a disorder in a patient, the disorder involving disregulation of EPO expression or activity, the method comprising administering to the patient a pharmaceutical composition as defined above.

The invention also relates to a method of treating, preventing or ameliorating the symptoms of a disorder in a patient, wherein the disorder is selected from the group consisting of: blood disorders characterized by low or defective red blood cell production, anemia, Chronic Renal Failure patients hypertension, surgery patients, Pediatric patients on dialysis, diseases or conditions associated with insufficient hematocrit levels, AIDS, disorders connected with chemotherapy treatments, cancers and tumors, infectious diseases, venereal diseases, immunologically related diseases and/or autoimmune diseases and disorders, cardiovascular diseases such as stroke, hypotension, cardiac arrest, ischemia in particular ischemia-reperfusion injury, myocardial infarction such as acute myocardial infarctions, chronic heart failure, angina, cardiac hypertrophy, cardiopulmonary diseases, heart-lung bypass, respiratory diseases, kidney, urinary and reproductive diseases, endocrine and metabolic abnormalities, gastrointestinal diseases, diseases of the central nervous system (CNS) or peripheral nervous system which have primarily neurological or psychiatric symptoms, age-related loss of cognitive function, cerebral palsy, neurodegenerative disease, Alzheimer's disease, Parkinson's disease, Leigh's disease, dementia, memory loss, amyotrophic lateral sclerosis, alcoholism, mood disorder, anxiety disorder, attention deficit disorder, hyperactivity, autism, schizophrenia, depression, brain or spinal cord trauma or ischemia, Creutzfeld-Jakob disease, ophthalmic diseases, seizure disorder, multiple sclerosis, inflammation, radiation damage, macular degeneration, diabetic neuropathy, diabetic retinopathy, glaucoma, retinal ischemia, and retinal trauma, the method comprising administering to the patient a therapeutically effective amount of a polypeptide or a pharmaceutical composition as defined above.

The invention further resides in the use of a polypeptide as defined above or of a pharmaceutical composition as defined above in the manufacture of a medicament for the treatment of a disorder in a patient, the disorder being selected from the group consisting of: blood disorders characterized by low or defective red blood cell production, anemia, Chronic Renal Failure patients hypertension, surgery patients, Pediatric patients on dialysis, diseases or conditions associated with insufficient hematocrit levels, AIDS, disorders connected with chemotherapy treatments, cancers and tumors, infectious diseases, venereal diseases, immunologically related diseases and/or autoimmune diseases and disorders, cardiovascular diseases such as stroke, hypotension, cardiac arrest, ischemia in particular ischemia-reperfusion injury, myocardial infarction such as acute myocardial infarctions, chronic heart failure, angina, cardiac hypertrophy, cardiopulmonary diseases, heart-lung bypass, respiratory diseases, kidney, urinary and reproductive diseases, endocrine and metabolic abnormalities, gastrointestinal diseases, diseases of the central nervous system (CNS) or peripheral nervous system which have primarily neurological or psychiatric symptoms, age-related loss of cognitive function, cerebral palsy, neurodegenerative disease, Alzheimer's disease, Parkinson's disease, Leigh's disease, dementia, memory loss, amyotrophic lateral sclerosis, alcoholism, mood disorder, anxiety disorder, attention deficit disorder, hyperactivity, autism, schizophrenia, depression, brain or spinal cord trauma or ischemia, Creutzfeld-Jakob disease, ophthalmic diseases, seizure disorder, multiple sclerosis, inflammation, radiation damage, macular degeneration, diabetic neuropathy, diabetic retinopathy, glaucoma, retinal ischemia, and retinal trauma.

A further object of this invention also resides in a pharmaceutical composition comprising an antibody, or a fragment or a derivative thereof as described here above, and a pharmaceutically acceptable carrier, excipient, or stabilizer.

The invention also relates to a method of treating, preventing or ameliorating the symptoms of a cancer in a subject, the method comprising administering to the patient an effective amount of an antibody, or a fragment or a derivative thereof as described here above. The invention also resides in the use of an antibody, or a fragment or a derivative thereof as described here above, in the manufacture of a medicament for the treatment of a cancer.

Other aspects of this invention include primers and probes specific for a nucleic acid as defined above, as well as their uses to detect or diagnose the presence of such a nucleic acid in a sample.

### LEGEND TO THE FIGURES

FIG. 1: Genomic sequence of 4099 nucleotides, which represent the human reference wild-type EPO gene region (SEQ ID NO: 1). The EPO gene has been described as containing five exons whose positions on the nucleotide sequence of FIG. 1 are the following: Exon 1: from nucleotide 601 to nucleotide 794 (comprises the start codon at position 782), Exon 2: from nucleotide 1359 to nucleotide 1504, Exon 3: from nucleotide 1763 to nucleotide 1849, Exon 4: from nucleotide 2465 to nucleotide 2644, Exon 5: from nucleotide 2779 to nucleotide 3499 (comprises the stop codon at position 2763). Each exon is coloured in grey. Start and stop codon are underlined.

FIG. 2: The transcript sequence of 1328 nucleotides of wild-type EPO is presented (excluding the polyA tail) (SEQ ID NO: 2). The start and stop codons, respectively at position 182 and 761, have been underlined.

FIG. 3: The transcript sequence of wild-type EPO as well as the encoded protein are presented. The start and stop codons, are at position 182 and 761 respectively. The transcript encodes an immature protein of 193 amino acids (named hereafter EPOwt) (SEQ ID NO: 3).

FIG. 4: The transcript sequence of EPOv1 as well as the encoded protein EPOv1 are presented. The start and stop codons, are at position 182 and 674 respectively (the coding sequence is therefore from position 182 to 676 including both the start and stop codons). The transcript encodes an immature protein of 164 amino acids (named hereafter EPOv1) (SEQ ID NO: 4). EPOv1 is a novel transcriptional variant of EPO, which is encoded by exons 1, 2, 4 and 5 of the human gene EPO.

FIG. 5: The transcript sequence of EPOv2 as well as the encoded protein EPOv2 are presented. The start and stop codons, are at position 182 and 494 respectively (the coding sequence is therefore from position 182 to 496 including both the start and stop codons). The transcript encodes an immature protein of 164 amino acids (named hereafter EPOv2) (SEQ ID NO: 6). EPOv2 is a novel transcriptional variant of EPO, which is encoded by exons 1, 2 and 5 of the human gene EPO.

FIG. 6: The transcript sequence of EPOv3 as well as the encoded protein EPOv3 are presented. The start and stop codons, are at position 182 and 644 respectively (the coding sequence is therefore from position 182 to 646 including both the start and stop codons). The transcript encodes an immature protein of 154 amino acids (named hereafter EPOv3) (SEQ ID NO: 9). EPOv3 is a novel transcriptional variant of EPO, which is encoded by exons 1, 2, 3 and 4A of the human gene EPO. The polypeptide having the sequence set forth at SEQ ID NO: 8 corresponds to the C-terminal part of the novel transcriptional variant of EPO disclosed here for the first time and is encoded by the 3′ end of exon 4A. Exon 4A is longer at the 3′ end as compared to exon 4 which encode for the wild-type EPO (see FIG. 3).

FIG. 7: The transcript sequence of EPOv as well as the encoded protein EPOv are presented. The start and stop codons, are at position 182 and 347 respectively (the coding sequence is therefore from position 182 to 349 including both the start and stop codons). The transcript encodes an immature protein of 55 amino acids (named hereafter EPOv) (SEQ ID NO: 13). EPOv is a synthetic truncated variant of EPO, which is encoded by exons 1, 2, and the first 6 nucleotides of exon 3 of the human gene EPO. In its mature form (EPOvm), it corresponds to the N-terminal 28 amino acids of the EPO mature polypeptide which form the first alpha helical motif.

FIG. 8: The sequence of a mutant of EPOv (EPOv C34S mutant) is presented. In this mutant, the free cysteine residue at position 7 of the mature protein (position 34 of the unmature form) is replaced by a serine. The transcript encodes an immature protein of 55 amino acids (named hereafter EPOv C34S) (SEQ ID NO: 15).

FIG. 9: Effect of electroporated Epo and Epo variant cDNAs on the CMAP at times after nerve crush. Groups of 6 female C57BL/6 mice were injected with the pDEST12.2 expression vector containing cDNAs for: EPO wild type (dots) or EPOv1 (horizontal-lines), EPOv2 (diagonal lines), EPOv3 (open squares), and EPOv (cross-hatching). As negative controls the pDEST12.2 was electroporated alone (black bars) or containing the cDNA for human IL4 (checkered bars). On days 7 and 14 following nerve crush the CMAP parameters latency, duration and amplitude were recorded in the crushed leg of all animals, and also in the contralateral leg of the animals injected with the pDEST12.2 vector alone (empty symbols). Statistical analysis is performed using the Mann-Whitney test.

FIG. 10: Effect of Epo variants on red blood cell count (haematocrit). Blood samples were taken on day 12 following electroporation from all animals described in FIG. 9. The haematocrit (red blood cell volume estimated as a percentage of the total blood volume) was determined.

FIG. 11: Effects of Epo variants on growth of TF-1. TF-1 cells (ATCC #CRL-2003) were cultured in 6-well cell culture dishes in the medium as recommended by ATCC, and supplemented either with 1 ng/ml GM-CSF or with the indicated doses of Eprex (top panel), rEPOwtm-6His (middle panel) or rEPOv1m (bottom panel). Cell cultures were set up at approximately 5×10⁴ cells/ml and the number of viable cells was estimated every day over a 4 days period, by counting duplicate chambers of a Neubauer Improved haemocytometer.

FIG. 12: Effect of injected EpoWT and Epo variant protein on the CMAP at times after nerve crush. Groups of 6 female C57BL/6 mice were injected with: 50 μg/kg Eprex (checkered bars), 52.4 μg/kg recombinant EPOwtm (dots), 43.5 μg/Kg EPOv1m (horizontal-lines), or 10.7 μg/Kg EPOvm C34S (cross-hatching), or 10.7 μg/Kg EPOv C34S shuffled (vertical-lines). As negative control, vehicle (PBS) was injected alone (black bars). On days 8 and 15 following nerve crush the CMAP parameters latency, duration and amplitude were recorded in the crushed leg of all animals, and also in the contralateral leg of the animals injected with vehicle alone (empty symbols). Statistical analysis is performed using the Mann-Whitney test.

FIG. 13: MBP content of sciatic nerves following nerve crush. On day 16 of the experiment described in FIG. 12, animals were sacrificed and the section ofthe sciatic nerve distal to the crush site was removed. The corresponding section of nerve from the contralateral leg of each animal was also removed and processed in parallel. Protein content of each sample was determined by the BCA method; MBP content was determined by Elisa. The MBP content of each sample was expressed as ngMBP/μg total protein and the MBP content of the crushed nerves was normalized to the MBP content of the corresponding contralateral nerve (% ContraL). Statistical analysis was performed using the One-Way ANOVA test. Groups are designated as follows: Vehicle (solid bars), Eprex (checkered bars), recombinant EPOwtm (stipled bars), EPOv1m (horizontal-lines), EPOvm C34S (dots), EPOv C34S shuffled (vertical-lines).

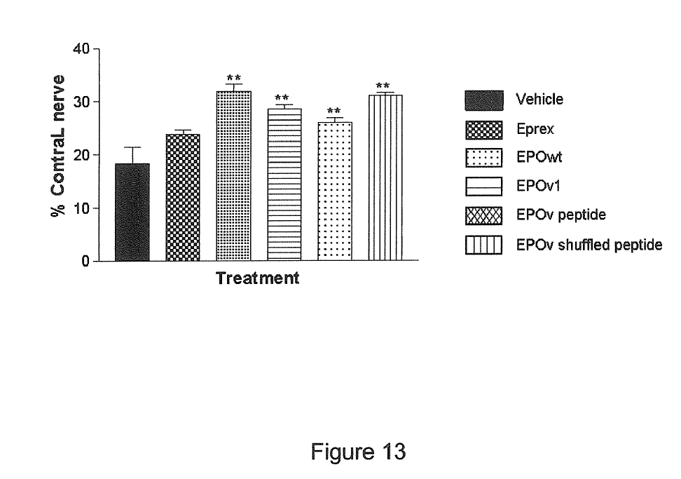

### DETAILED DESCRIPTION OF THE INVENTION

The present invention results in part from the identification, isolation and characterization of novel transcriptional variants of EPO having particular structural and biological properties. These transcriptional variants and derivatives thereof represent valuable pharmaceutical products. The present invention results also in part from the identification of the domain of EPO having tissue protective activity (in particular neurotrophic activity) and the identification of EPO variants having tissue protective activity (in particular neurotrophic activity) but lacking an hematotrophic activity.

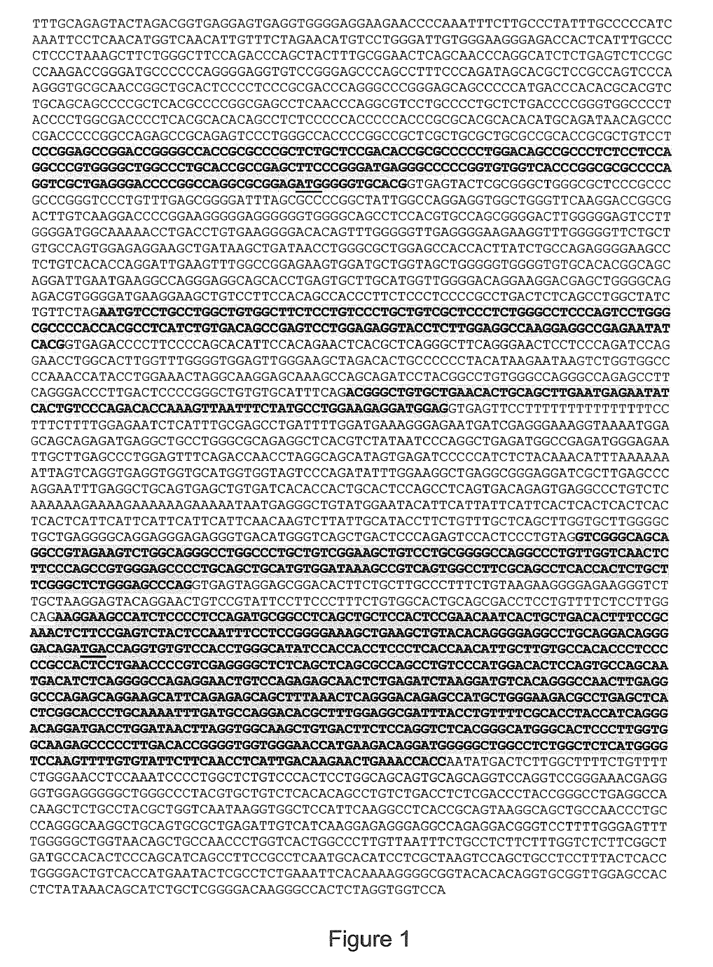

A genomic sequence of 4099 nucleotides, which represent the human reference wild-type EPO gene region, is presented at FIG. 1 (SEQ ID NO: 1). The EPO gene has been described as containing five exons whose positions on the nucleotide sequence SEQ ID NO: 1 are the following:
Exon 1: from nucleotide 601 to nucleotide 794 (comprises the start codon at position 782).
Exon 2: from nucleotide 1359 to nucleotide 1504.
Exon 3: from nucleotide 1763 to nucleotide 1849.
Exon 4: from nucleotide 2465 to nucleotide 2644.
Exon 5: from nucleotide 2779 to nucleotide 3499 (comprises the stop codon at position 2763).

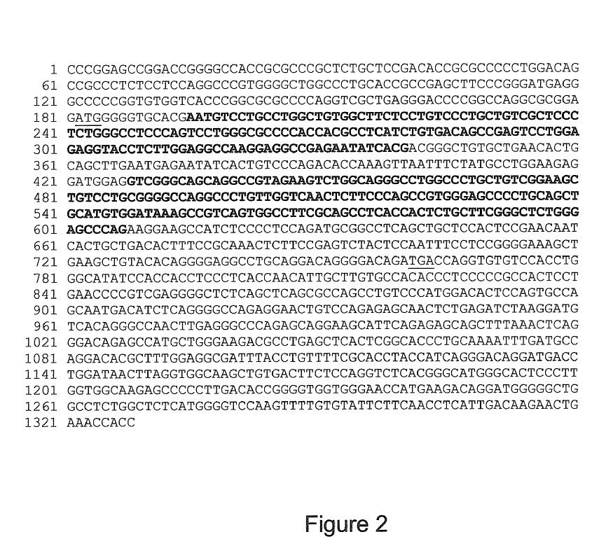

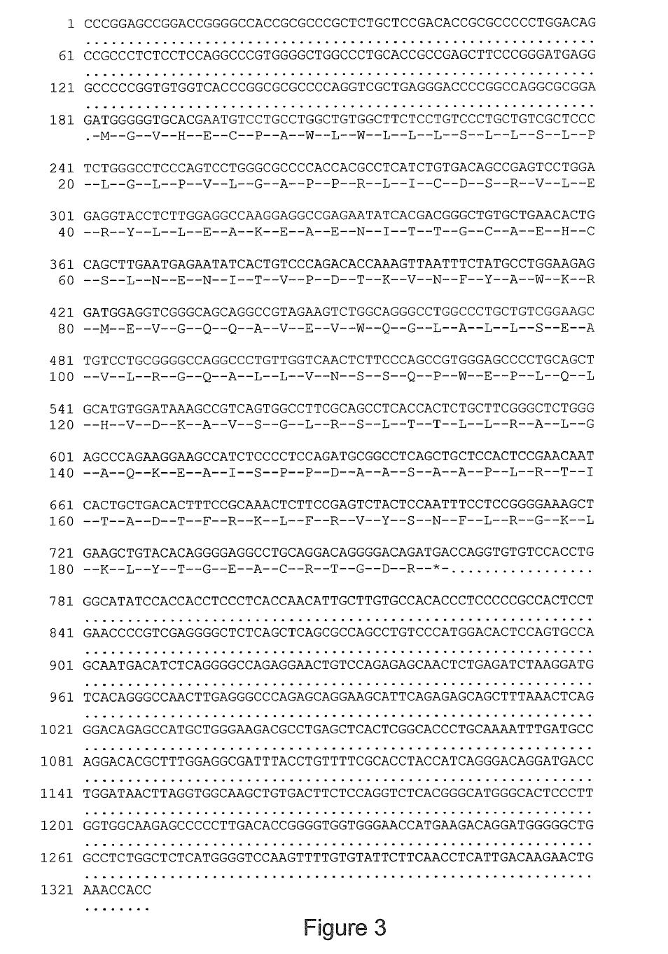

The corresponding transcript is presented on FIG. 2 (excluding the polyA tail). The start and stop codons, respectively at position 182 and 761, have been underlined. This transcript encodes an immature protein of 193 amino acids (named hereafter EPOwt) as shown in FIG. 3 (SEQ ID NO: 3). This immature protein is processed and the N-terminal signal peptide that includes the first 27 amino acids is cleaved. The resulting protein is 166 amino acids long and is named hereafter EPOwtm (amino acid 28 to 193 of SEQ ID NO: 3). Moreover, it has been described that the carboxyl-terminal residue is removed such that the protein expressed by the cells is a 165 amino acid long protein (amino acid 28 to 192 of SEQ ID NO: 3).

Erythropoietin has an up-up-down-down four-helical bundle topology with interhelical angles similar to those of the long-chain class, for example hGH and granulocyte colony-stimulating factor (see Syed R S et al., Nature 395 (6701):511-6 (1998)). However, it also contains two small antiparallel β-strands typical of the short-chain class, for example macrophage colony-stimulating factor, stem-cell factor, interleukin-4 and interleukin-5. One pair of antiparallel long helices, αA (residues 8-26 of EPOwtm) and αD (residues 138-161 of EPOwtm), is held together by a disulphide bridge, Cys 7 to Cys 161. The other pair, αB (residues 55-83 of EPOwtm) and αC (residues 90-112 of EPOwtm), is linked by a short loop. The αD helix is slightly irregular because of a small kink at Gly 151. The short segments of amino acids from the long AB and CD crossover loops interact with each other to form an antiparallel β-sheet: β1 (residues 39-41 of EPOwtm) and β2 (residues 133-135 of EPOwtm). Several aromatic and hydrophobic residues, such as Phe 138, Phe 142, Tyr 145, Phe 148, Leu 153 and Tyr 156, on the interior face of the D-helix, pack against the non-polar side chains from the A, B and C helices to form the hydrophobic core of erythropoietin. A second disulphide bond, Cys 29 to Cys 33, links the end of the αA helix with part of the AB loop. Erythropoietin has two additional short helices, the αB′ helix (residues 47-52 of EPOwtm) orthogonal to αB and the mini-helix αC′ (residues 114-121 of EPOwtm) following αC with a 90° tilt beginning at Gly 113.

Both human urinary derived EPO (Miyake et al. J. Biol. Chem. 252, 5558 (1977)) and recombinant human EPO expressed in mammalian cells contain three N-linked and one 0-linked oligosaccharide chains which together comprise about 40% of the total molecular weight of the glycoprotein. N-linked glycosylation occurs at asparagine residues located at positions 24, 38 and 83 of EPOwtm, while 0-linked glycosylation occurs at a serine residue located at position 126 (Lai et al. J. Biol. Chem.261, 3116 (1986); Broudy et al. Arch. Biochem. Biophys. 265, 329(1988)). The oligosaccharide chains have been shown to be modified with terminal sialic acid residues with N-linked chains typically having up to four sialic acids per chain and 0-linked chains having up to two sialic acids. An EPO polypeptide may therefore accommodate up to a total of 14 sialic acids. Various studies have shown that alterations of EPO carbohydrate chains can affect biological activity. For example, it has been shown that enzymatic removal of all sialic acid residues from the glycosylated erythropoietin results in loss of in vivo activity but not in vitro activity because sialylation of erythropoietin prevents its binding, and subsequent clearance, by hepatic binding protein.

The applicant has now identified novel transcriptional variants of human EPO. These transcriptional variants and derivatives thereof represent valuable pharmaceutical products.

1. EPO Polypeptides and Variants of the Present Invention:

The inventors of the present invention have identified the domain of EPO having tissue protective activity (in particular neurotrophic activity) and EPO variants having tissue protective activity (in particular neurotrophic activity) but lacking an hematotrophic activity. These short versions of EPO and the EPO variants identified represent valuable pharmaceutical products.

1.1 EPOshort Polypeptides and Variants Thereof:

In a first aspect, the invention resides in an isolated erythropoietin variant polypeptide, said variant polypeptide consisting of a polypeptide differing solely from the sequence set forth at SEQ ID NO: 3 by the lack of at least one, preferably at least two, more preferably at least three, even more preferably at least four, even more preferably at least five, even more preferably at least six, even more preferably at least seven, even more preferably at least eight, even more preferably at least nine, even more preferably at least ten, even more preferably at least eleven, even more preferably at least twelve, even more preferably at least thirteen, even more preferably at least fourteen, even more preferably at least fifteen, even more preferably at least sixteen, even more preferably at least seventeen, even more preferably at least eighteen, even more preferably at least nineteen, even more preferably at least twenty, even more preferably at least twenty-one, even more preferably at least twenty-two, even more preferably at least twenty-three, even more preferably at least twenty-four, even more preferably at least twenty-five, even more preferably at least twenty-six, even more preferably at least twenty-seven, even more preferably at least twenty-eight, even more preferably at least twenty-nine, even more preferably at least thirty, even more preferably at least thirty-one, even more preferably at least thirty-two, even more preferably at least thirty-three, even more preferably at least thirty-four, even more preferably at least thirty-five, even more preferably at least thirty-six, even more preferably at least thirty-seven, even more preferably at least thirty-eight, even more at least preferably thirty-nine, even more preferably at least forty, even more preferably at least forty-one, even more preferably at least forty-two, even more preferably at least forty-three, even more preferably at least forty-four, even more preferably at least forty-five, even more preferably at least forty-six, even more preferably at least forty-seven, even more preferably at least forty-eight, even more preferably at least forty-nine, even more preferably at least fifty, even more preferably at least fifty-one, even more preferably at least fifty-two, even more preferably at least fifty-three, even more preferably at least fifty-four, even more preferably at least fifty-five, even more preferably at least fifty-six, even more preferably at least fifty-seven, even more preferably at least fifty-eight, even more preferably at least fifty-nine, even more preferably at least sixty, even more preferably at least sixty-one, even more preferably at least sixty-two, even more preferably at least sixty-three, even more preferably at least sixty-four, even more preferably at least sixty-five, even more preferably at least sixty-six, even more preferably at least sixty-seven, even more preferably at least sixty-eight, even more preferably at least sixty-nine, even more preferably at least seventy, even more preferably at least seventy-one, even more preferably at least seventy-two, even more preferably at least seventy-three, even more preferably at least seventy-four, even more preferably at least seventy-five, even more preferably at least seventy-six, even more preferably at least seventy-seven, even more preferably at least seventy-eight, even more preferably at least seventy-nine, even more preferably at least eighty, even more preferably at least eighty-one, even more preferably at least eighty-two, even more preferably at least eighty-three, even more preferably at least eighty-four, even more preferably at least eighty-five, even more preferably at least eighty-six, even more preferably at least eighty-seven, even more preferably at least eighty-eight, even more preferably at least eighty-nine, even more preferably at least ninety, even more preferably at least ninety-one, even more preferably at least ninety-two, even more preferably at least ninety-three, even more preferably at least ninety-four, even more preferably at least ninety-five, even more preferably at least ninety-six, even more preferably at least ninety-seven, even more preferably at least ninety-eight, even more preferably at least ninety-nine, even more preferably at least one hundred, even more preferably at least one hundred and one, even more preferably at least one hundred and two, even more preferably at least one hundred and three, even more preferably at least one hundred and four, even more preferably at least one hundred and five, even more preferably at least one hundred and six, even more preferably at least one hundred and seven, even more preferably at least one hundred and eight, even more preferably at least one hundred and nine, even more preferably at least one hundred and ten, even more preferably at least one hundred and eleven, even more preferably at least one hundred and twelve, even more preferably at least one hundred and thirteen, even more preferably at least one hundred and fourteen, even more preferably at least one hundred and fiveteen, even more preferably at least one hundred and sixteen, even more preferably at least one hundred and seventeen, even more preferably at least one hundred and eighteen, even more preferably at least one hundred and nineteen, even more preferably at least one hundred and twenty, even more preferably at least one hundred and twenty one, even more preferably at least one hundred and twenty two, even more preferably at least one hundred and twenty three, even more preferably at least one hundred and twenty four, even more preferably at least one hundred and twenty five, even more preferably at least one hundred and twenty six, even more preferably at least one hundred and twenty seven, even more preferably at least one hundred and twenty eight, even more preferably at least one hundred and twenty nine, even more preferably at least one hundred and thirty, even more preferably at least one hundred and thirty one, even more preferably at least one hundred and thirty two, even more preferably at least one hundred and thirty three, even more preferably at least one hundred and thirty four, even more preferably at least one hundred and thirty five, even more preferably at least one hundred and thirty six, even more preferably at least one hundred and thirty seven, and most preferably one hundred and thirty eight amino acids of the amino acids 56 (Cysteine) to 193 (Arginine) of SEQ ID NO: 3.

In a particular embodiment, the invention resides in an isolated erythropoietin variant polypeptide, said variant polypeptide consisting of a polypeptide differing solely from the sequence set forth at SEQ ID NO: 3 by the lack of at least one hundred of the amino acids 56 (Cysteine) to 193 (Arginine) of SEQ ID NO: 3. In another particular embodiment, the invention resides in an isolated erythropoietin variant polypeptide, said variant polypeptide consisting of a polypeptide differing solely from the sequence set forth at SEQ ID NO: 3 by the lack of at least one hundred and twenty or at least one hundred and thirty or at least one hundred and thirty five, of the amino acids 56 (Cysteine) to 193 (Arginine) of SEQ ID NO: 3. In another particular embodiment, the invention resides in an isolated erythropoietin variant polypeptide, said variant polypeptide consisting of a polypeptide differing solely from the sequence set forth at SEQ ID NO: 3 by the lack of at least one hundred and thirty six or at least one hundred and thirty seven, of the amino acids 56 (Cysteine) to 193 (Arginine) of SEQ ID NO: 3. In another particular embodiment the erythropoietin variant polypeptide has the sequence set forth at SEQ ID NO: 13 (named here after EPOv). The term “isolated” when used to describe the various polypeptides disclosed herein, means polypeptide that has been identified and separated and/or recovered from a component of its natural environment. Isolated products of this invention may thus be contained in a culture supernatant, partially enriched or purified, produced from heterologous sources, cloned in a vector or formulated with a vehicle, etc.

In yet another particular embodiment, the invention resides in an isolated erythropoietin variant polypeptide, said polypeptide consisting of a polypeptide differing solely from the sequence set forth at SEQ ID NO: 3 by the lack of amino acid 193 (Arginine), preferably the lack of amino acids 192-193, more preferably the lack of amino acids 191-193, even more preferably the lack of amino acids 190-193, even more preferably the lack of amino acids 189-193, even more preferably the lack of amino acids 188-193, even more preferably the lack of amino acids 187-193, even more preferably the lack of amino acids 186-193, even more preferably the lack of amino acids 185-193, even more preferably the lack of amino acids 184-193, even more preferably the lack of amino acids 183-193, even more preferably the lack of amino acids 182-193, even more preferably the lack of amino acids 181-193, even more preferably the lack of amino acids 180-193, even more preferably the lack of amino acids 179-193, even more preferably the lack of amino acids 178-193, even more preferably the lack of amino acids 177-193, even more preferably the lack of amino acids 176-193, even more preferably the lack of amino acids 175-193, even more preferably the lack of amino acids 174-193, even more preferably the lack of amino acids 173-193, even more preferably the lack of amino acids 172-193, even more preferably the lack of amino acids 171-193, even more preferably the lack of amino acids 170-193, even more preferably the lack of amino acids 169-193, even more preferably the lack of amino acids 168-193, even more preferably the lack of amino acids 167-193, even more preferably the lack of amino acids 166-193, even more preferably the lack of amino acids 165-193, even more preferably the lack of amino acids 164-193, even more preferably the lack of amino acids 163-193, even more preferably the lack of amino acids 162-193, even more preferably the lack of amino acids 161-193, even more preferably the lack of amino acids 160-193, even more preferably the lack of amino acids 159-193, even more preferably the lack of amino acids 158-193, even more preferably the lack of amino acids 157-193, even more preferably the lack of amino acids 156-193, even more preferably the lack of amino acids 155-193, even more preferably the lack of amino acids 154-193, even more preferably the lack of amino acids 153-193, even more preferably the lack of amino acids 152-193, even more preferably the lack of amino acids 151-193, even more preferably the lack of amino acids 150-193, even more preferably the lack of amino acids 149-193, even more preferably the lack of amino acids 148-193, even more preferably the lack of amino acids 147-193, even more preferably the lack of amino acids 146-193, even more preferably the lack of amino acids 145-193, even more preferably the lack of amino acids 144-193, even more preferably the lack of amino acids 143-193, even more preferably the lack of amino acids 142-193, even more preferably the lack of amino acids 141-193, even more preferably the lack of amino acids 140-193, even more preferably the lack of amino acids 139-193, even more preferably the lack of amino acids 138-193, even more preferably the lack of amino acids 137-193, even more preferably the lack of amino acids 136-193, even more preferably the lack of amino acids 135-193, even more preferably the lack of amino acids 134-193, even more preferably the lack of amino acids 133-193, even more preferably the lack of amino acids 132-193, even more preferably the lack of amino acids 131-193, even more preferably the lack of amino acids 130-193, even more preferably the lack of amino acids 129-193, even more preferably the lack of amino acids 128-193, even more preferably the lack of amino acids 127-193, even more preferably the lack of amino acids 126-193, even more preferably the lack of amino acids 125-193, even more preferably the lack of amino acids 124-193, even more preferably the lack of amino acids 123-193, even more preferably the lack of amino acids 122-193, even more preferably the lack of amino acids 121-193, even more preferably the lack of amino acids 120-193, even more preferably the lack of amino acids 119-193, even more preferably the lack of amino acids 118-193, even more preferably the lack of amino acids 117-193, even more preferably the lack of amino acids 116-193, even more preferably the lack of amino acids 115-193, even more preferably the lack of amino acids 114-193, even more preferably the lack of amino acids 113-193, even more preferably the lack of amino acids 112-193, even more preferably the lack of amino acids 111-193, even more preferably the lack of amino acids 110-193, even more preferably the lack of amino acids 109-193, even more preferably the lack of amino acids 108-193, even more preferably the lack of amino acids 107-193, even more preferably the lack of amino acids 106-193, even more preferably the lack of amino acids 105-193, even more preferably the lack of amino acids 104-193, even more preferably the lack of amino acids 103-193, even more preferably the lack of amino acids 102-193, even more preferably the lack of amino acids 101-193, even more preferably the lack of amino acids 100-193, even more preferably the lack of amino acids 99-193, even more preferably the lack of amino acids 98-193, even more preferably the lack of amino acids 97-193, even more preferably the lack of amino acids 96-193, even more preferably the lack of amino acids 95-193, even more preferably the lack of amino acids 94-193, even more preferably the lack of amino acids 93-193, even more preferably the lack of amino acids 92-193, even more preferably the lack of amino acids 91-193, even more preferably the lack of amino acids 90-193, even more preferably the lack of amino acids 89-193, even more preferably the lack of amino acids 88-193, even more preferably the lack of amino acids 87-193, even more preferably the lack of amino acids 86-193, even more preferably the lack of amino acids 85-193, even more preferably the lack of amino acids 84-193, even more preferably the lack of amino acids 83-193, even more preferably the lack of amino acids 82-193, even more preferably the lack of amino acids 81-193, even more preferably the lack of amino acids 80-193, even more preferably the lack of amino acids 79-193, even more preferably the lack of amino acids 78-193, even more preferably the lack of amino acids 77-193, even more preferably the lack of amino acids 76-193, even more preferably the lack of amino acids 75-193, even more preferably the lack of amino acids 74-193, even more preferably the lack of amino acids 73-193, even more preferably the lack of amino acids 72-193, even more preferably the lack of amino acids 71-193, even more preferably the lack of amino acids 70-193, even more preferably the lack of amino acids 69-193, even more preferably the lack of amino acids 68-193, even more preferably the lack of amino acids 67-193, even more preferably the lack of amino acids 66-193, even more preferably the lack of amino acids 65-193, even more preferably the lack of amino acids 64-193, even more preferably the lack of amino acids 63-193, even more preferably the lack of amino acids 62-193, even more preferably the lack of amino acids 61-193, even more preferably the lack of amino acids 60-193, even more preferably the lack of amino acids 59-193, even more preferably the lack of amino acids 58-193, even more preferably the lack of amino acids 57-193, even more preferably the lack of amino acids 56-193. In yet another particular embodiment, the invention resides in an isolated erythropoietin variant polypeptide, said polypeptide consisting of a polypeptide differing solely from the sequence set forth at SEQ ID NO: 3 by the lack of amino acids 100-193. In yet another particular embodiment, the invention resides in an isolated erythropoietin variant polypeptide, said polypeptide consisting of a polypeptide differing solely from the sequence set forth at SEQ ID NO: 3 by the lack of amino acids 61-193. In yet another particular embodiment, the invention resides in an isolated erythropoietin variant polypeptide, said polypeptide consisting of a polypeptide differing solely from the sequence set forth at SEQ ID NO: 3 by the lack of amino acids 60-193. In yet another particular embodiment, the invention resides in an isolated erythropoietin variant polypeptide, said polypeptide consisting of a polypeptide differing solely from the sequence set forth at SEQ ID NO: 3 by the lack of amino acids 59-193. In yet another particular embodiment, the invention resides in an isolated erythropoietin variant polypeptide, said polypeptide consisting of a polypeptide differing solely from the sequence set forth at SEQ ID NO: 3 by the lack of amino acids 58-193. In yet another particular embodiment, the invention resides in an isolated erythropoietin variant polypeptide, said polypeptide consisting of a polypeptide differing solely from the sequence set forth at SEQ ID NO: 3 by the lack of amino acids 57-193. In a particular embodiment the isolated erythropoietin variant polypeptide has the sequence set forth at SEQ ID NO: 13 (named here after EPOv).

In a further preferred embodiment, the peptides described here above are mature peptide lacking the N-terminal signal peptide. More particularly, the peptides described here above lack the signal peptide consisting of amino acids 1 to 27 of SEQ ID NO: 3. Therefore, in a particular aspect, the invention resides in a polypeptide consisting of the sequence of amino acids 28 to 55 of SEQ ID NO: 13 (named here after EPOvm).

The polypeptides described here above in this section 1.1 will be named here after “EPOshort polypeptides”. In another aspect, the invention resides in an isolated polypeptide comprising an EPOshort polypeptide.

In a further aspect, the present invention resides in an isolated polypeptide comprising or consisting of a variant of the EPOshort polypeptides described hereabove. A variant of the EPOshort polypeptides being defined as polypeptides comprising one or several amino acid substitutions as compared to EPOshort polypeptides described hereabove, typically from 0 to 10 amino acid substitutions, even more typically from 0 to 5, 4, 3, 2 or 1 amino acid substitutions. In a particular embodiment, the variant polypeptide differs from the EPOshort polypeptides described hereabove by at least one, two, three, four, five, six, seven, eight, nine or ten mutations chosen in the group consisting of: E40Q, Q85QQ, G104S, L129G, L129P, L129S, S131N, L132F, SL131-132NF, T134D, G14OR and S147C. In a preferred embodiment, the variant polypeptide differs from the sequence of the EPOshort polypeptides described hereabove by one or two mutation chosen in the group consisting of: G104S and S147C. The notation used herein for modification of amino acid sequence means that the wild-type amino acid at the indicated position is changed to the amino acid that immediately follows the respective number. The numbering given is relative to the numbering of the amino acids at SEQ ID NO: 3. Thus for example, the E40Q mutation corresponds to a mutation of the amino acid E (Glutamic acid) at position 40 of SEQ ID NO: 3 into an amino acid Q (Glutamine). One or more of these mutation sites might however be absent depending on the number of amino acids that are missing in EPOshort polypeptide, compared to EPOwt. In another embodiment, the variant of the EPOshort polypeptides differs from the EPOshort polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A. In still another embodiment, the variant of the EPOshort polypeptides differs from the EPOshort polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A and N51K. In still another embodiment, a variant of the EPOshort polypeptides differs from the EPOshort polypeptides described hereabove by one of the combination mutations chosen in the group consisting of: K72D/S127E, A57N/H59T, K72D/R177E, R130E/L135S, K79A/K167A, K72A/K79A/K167A, K124A/K179A, K72A/K124A/K179A, K72A/K79A/K124A/K179A, K72A/K79A/K124A/K167A/K179A, K72A/K79A/K124A/K167A/K179A/K181A, N51K/N65K/N110K, and Y42A/N51K. In a preferred embodiment, a variant of the EPOshort polypeptides differs from the sequence of the EPOshort polypeptides described hereabove by the mutation consisting of C34S. One or more of these mutation sites might however be absent depending on the number of amino acids that are missing in EPOshort polypeptide, compared to EPOwt. In a preferred embodiment, the variant of the EPOshort peptides described here above are mature peptide lacking the N-terminal signal peptide. More particularly, the peptides described here above lack the signal peptide consisting of amino acids 1 to 27 of SEQ ID NO: 3. Therefore, in a particular aspect, the invention resides in a polypeptide consisting of an amnino acid sequence differing from EPOvm by the mutation C34S (amino acids 28 to 55 of SEQ ID NO: 15).

In another embodiment, the present invention resides in analogs of EPO polypeptides corresponding to an isolated polypeptide comprising or consisting of an EPOshort or a variant of the EPOshort polypeptides described hereabove, which differ in addition from such polypeptides such as having from 1 to 6 additional sites for glycosylation. Glycosylation of a protein, with one or more oligosaccharide groups, occurs at specific locations along a polypeptide backbone and affects the physical properties of the protein such as protein stability, secretion, subcellular localisation, and biological activity. Glycosylation is usually oftwo types: O-linked oligosaccharides are attached to serine or threonine residues and N-linked oligosaccharides are attached to asparagine residues. One type of oligosaccharide found on both N-linked and 0-linked oligosaccharides is N-acetylneuraminic acid (sialic acid), which is a family of amino sugars containing 9 or more carbon atoms. Sialic acid is usually the terminal residue on both N-linked and 0-linked oligosaccharides and, because it bears a negative charge, confers acidic properties to the glycoprotein. The polypeptides of the present invention include analogs of EPOshort or of a variant of the EPOshort polypeptides described hereabove with one or more changes in the amino acid sequence which result in an increase in the number of sites for sialic acid attachment. These glycoprotein analogs may be generated by site-directed mutagenesis having additions, deletions, or substitutions of amino acid residues that increase or alter sites that are available for glycosylation. EPO analogs of the present invention having levels of sialic acid greater than those found in human erythropoietin are generated by adding glycosylation sites which do not perturb the secondary or tertiary conformation required for biological activity. The polypeptides of the present invention also include EPO analogs having increased levels of carbohydrate attachment at a glycosylation site which usually involve the substitution of one or more amino acids in close proximity to an N-linked or 0-linked site. The polypeptides of the present invention also include EPO analogs having one or more amino acids extending from the carboxy terminal end of erythropoietin and providing at least one additional carbohydrate site. The polypeptides of the present invention also include EPO analogs having an amino acid sequence which includes a rearrangement of at least one site for glycosylation. Such a rearrangement of glycosylation site involves the deletion of one or more glycosylation sites in EPOshort or in a variant of the EPOshort polypeptides described hereabove and the addition of one or more non-naturally occurring glycosylation sites. Increasing the number of carbohydrate chains on erythropoietin, and therefore the number of sialic acids per erythropoietin molecules may confer advantageous properties such as increased solubility, greater resistance to proteolysis, reduced immunogenecity, increased serum half-life, and increased biological activity. Erythropoietin analogs with additional glycosylation sites are disclosed in more detail in European Patent Application 640 619, PCT application WO0024893 and WO0181405.

In a preferred embodiment, such EPO analogs of the present invention comprise or consist of the EPOshort polypeptide or of a variant of the EPOshort polypeptides described hereabove, which includes at least one additional N-linked glycosylation site at position 84, 96, 113, 115, 116 or 141. As already explained hereabove, the position given is relative to the numbering of the amino acids at SEQ ID NO: 3. One or more of these site might be absent depending on the number of amino acids that are missing in EPOshort, or in the variant of the EPOshort polypeptides, compared to EPOwt. In another embodiment, such EPO analogs includes at least two additional glycosylation sites, or at least three additional glycosylation sites, or at least four additional glycosylation sites.

In a preferred embodiment, these EPO analogs of the present invention comprise or consist of the EPOshort polypeptide or of a variant of the EPOshort polypeptides described hereabove, modified by a modification selected from the following:

G84N and Q86T;

L96N;

L96N and S98T;

A95S, L96N and S98T;

Q113N, P114V and W115T;

P114V, W115N and P117T;

P114V, W115N and P117S;

P114A, W115N and P117T;

P114S, W115N and P117T;

P114S W115N, E116G and P117T;

P114V, W115N, E116G and P117T;

P114S W115N, P117T and Q119T;

N110Q, P114S, W115N and P117T;

P114S W115N, P117T, R189A;

L96N, S98T, P114S, W115N and P117T;

E116N, P117I and L118T;

P114S, E116N, P117I and L118T;

A141N;

A141N and K143T;

P114V, W115N, P117T, A141N and K143T;

A151P and A152T;

A152T;

A152N and A154S;

D163N and F165T;

F165N and K167T.

As already mentioned hereabove, the position given is relative to the numbering of the amino acids at SEQ ID NO: 3. One or more of these sites might be absent depending on the number of amino acids that are missing in EPOshort, or in the variant of the EPOshort polypeptides, compared to EPOwt.

In a further aspect, the present invention resides in an isolated polypeptide comprising or consisting of a homolog of an EPOshort polypeptide, or a variant of said EPOshort polypeptide or an analog of EPO polypeptides described here above in this section 1.1. In a particular embodiment, said homolog is defined as an active polypeptide having at least 80% amino acid sequence identity with the EPOshort polypeptide, or the variant of said EPOshort polypeptide or the analog of EPO polypeptides. Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide is from 180 to 28 amino acids in length, from 140 to 28 amino acids in length, often from 100 to 28 amino acids in length, often from 75 to 28 amino acids in length, more often from 55 to 28 amino acids in length, more from 40 to 28 amino acids in length, more often 30 amino acids in length, more often 29 amino acids in length, more often 28 amino acids in length. In a particular embodiment, said homolog consists or comprises an active polypeptide having at least 80% amino acid sequence identity with the polypeptide set forth at SEQ ID NO: 13 (EPOv) or the polypeptide having the sequence of amino acids 28 to 55 of SEQ ID NO: 13 (EPOvm). Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide of EPOv is 60 amino acids in length, more often 59 amino acids in length, more often 58 amino acids in length, more often 57 amino acids in length, more often from 56 amino acids in length, more often 55 amino acids in length. Ordinarily, the homolog polypeptide of EPOvm is 35 amino acids in length, more often 34 amino acids in length, more often 33 amino acids in length, more often 32 amino acids in length, more often from 31 amino acids in length, more often 30 amino acids in length, more often 29 amino acids in length, more often 28 amino acids in length.

“Percent (%) amino acid sequence identity” with respect to the EPO polypeptide sequences identified herein is defined as the percentage of amino acid residues in a candidate sequence that are identical with the amino acid residues in the specific EPO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol Biol. (1990). 215 (3): 403-410). Those skilled in the art can determine appropriate parameters for measuring alignment, including any algorithms needed to achieve maximal alignment over the full length of the sequences being compared.

1.2 EPOshort1 Polypeptides and Variants Thereof:

In a further aspect, the invention resides in an isolated polypeptide consisting of an EPO polypeptide differing from the sequence set forth at SEQ ID NO: 3 by the lack of at least one, preferably at least two, more preferably at least three, even more preferably at least four, even more preferably at least five, even more preferably at least six, even more preferably at least seven, even more preferably at least eight, even more preferably at least nine, even more preferably at least ten, even more preferably at least eleven, even more preferably at least twelve, even more preferably at least thirteen, even more preferably at least fourteen, even more preferably at least fifteen, even more preferably at least sixteen, even more preferably at least seventeen, even more preferably at least eighteen, even more preferably at least nineteen, even more preferably at least twenty, even more preferably at least twenty-one, even more preferably at least twenty-two, even more preferably at least twenty-three, even more preferably at least twenty-four, even more preferably at least twenty-five, even more preferably at least twenty-six, even more preferably at least twenty-seven, even more preferably at least twenty-eight and most preferably twenty-nine amino acids of the amino acids 54 (Threonine) to 82 (Glutamic acid) of SEQ ID NO: 3. These EPO polypeptides will be named “EPOshort1” here after. In another aspect, the invention resides in an isolated polypeptide comprising an EPOshort1 polypeptide.

The term “isolated” when used to describe the various polypeptides disclosed herein, means polypeptide that has been identified and separated and/or recovered from a component of its natural environment. Isolated products of this invention may thus be contained in a culture supernatant, partially enriched or purified, produced from heterologous sources, cloned in a vector or formulated with a vehicle, etc.

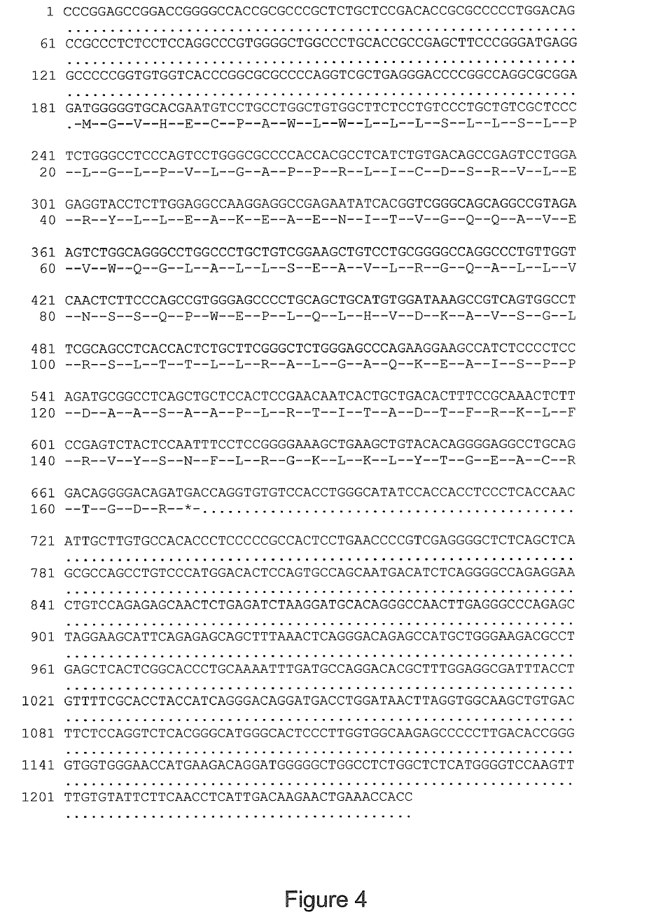

In a preferred embodiment, a polypeptide according to the present invention have the sequence set forth at SEQ ID NO: 4 (named here after EPOv1) and corresponds to a novel transcriptional variant of EPO. EPOv1 is encoded by exons 1, 2, 4 and 5 of the human gene EPO and lacks the 29 amino acids encoded by exon 3 (see FIG. 4). This transcriptional variant is therefore 164 amino acids long in its immature form. The N-terminal signal peptide includes the first 27 amino acids. Once the signal peptide is cleaved, the resulting protein is 137 amino acids long and is named hereafter EPOv1m (amino acid 28 to 164 of SEQ ID NO: 4). As already mentioned hereabove, the removal of the carboxyl-terminal residue has been described for EPOwtm such that the protein expressed by the cells is a 165 amino acid long protein (amino acid 28 to 192 of SEQ ID NO: 3). Therefore, the mature EPOv1m protein might be processed in the same manner by the cell and the resulting protein might be 136 amino acids long (amino acid 28 to 163 of SEQ ID NO: 4). More generally, in an embodiment of the present invention, the EPOshort1 polypeptides described hereabove lacks this carboxyl-terminal Arginine residue.

As described here above, EPOv1 (presented at FIG. 4 and SEQ ID NO: 4) lacks amino acids 54 to 82 of EPOwt which are encoded by exon 3. It can be concluded that EPOv1 retains the antiparallel long helices αA (residues 8-26 of EPOwtm), αC (residues 90-112 of EPOwtm), αD helix (residues 138-161 of EPOwtm) and a large part of αB (residues 55-83 of EPOwtm). The antiparallel β-sheet: β2 (residues 133-135 of EPOwtm) is also present, but the antiparallel β-sheet: β1 (residues 39-41 of EPOwtm) is absent of EPOv1m. The disulphide bond, Cys 29 to Cys 33 is absent. The mini-helix αC′ (residues 114-121 of EPOwtm) is retained in EPOv1m but the short helice αB′ helix (residues 47-52 of EPOwtm) is absent. The disulphide bond, Cys 7 to Cys 161, which links together αA helix and and αD should also be retained.

In a further aspect, the present invention resides in an isolated polypeptide comprising or consisting of a variant of the EPOshort1 polypeptides described hereabove. A variant being defined as polypeptides comprising one or several amino acid substitutions as compared to the EPOshort1 polypeptides described hereabove, typically from 0 to 10 amino acid substitutions, even more typically from 0 to 5, 4, 3, 2 or 1 amino acid substitutions. In a particular embodiment, the variant polypeptide differs from the EPOshort1 polypeptides described hereabove by at least one, two, three, four, five, six, seven, eight, nine or ten mutations chosen in the group consisting of: E40Q, Q85QQ, G104S, L129G, L129P, L129S, S131N, L132F, SL131-132NF, T134D, G140R and S147C. In a preferred embodiment, the variant polypeptide differs from the sequence of the EPOshort1 polypeptides described hereabove by one or two mutation chosen in the group consisting of: G104S and S147C. In another embodiment, the variant of the EPOshort1 polypeptides differs from the EPOshort1 polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A. In still another embodiment, the variant of the EPOshort1 polypeptides differs from the EPOshort1 polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A. In still another embodiment, a variant of the EPOshort1 polypeptides differs from the EPOshort1 polypeptides described hereabove by one of the combination mutations chosen in the group consisting of: K72D/S127E, A57N/H59T, K72D/R177E, R130E/L135S, K79A/K167A, K72A/K79A/K167A, K124A/K179A, K72A/K124A/K179A, K72A/K79A/K124A/K179A, K72A/K79A/K124A/K167A/K179A, K72A/K79A/K124A/K167A/K179A/K181A, N51K/N65K/N110K, and Y42A/N51K. In a preferred embodiment, the variant polypeptide differs from the sequence of the EPOshort1 polypeptides described hereabove by the mutation consisting of C34S. The notation used herein for modification of amino acid sequence means that the wild-type amino acid at the indicated position is changed to the amino acid that immediately follows the respective number. The numbering given is relative to the numbering of the amino acids at SEQ ID NO: 3. Thus for example, the E40Q mutation corresponds to a mutation of the amino acid E (Glutamic acid) at position 40 of SEQ ID NO: 3 into an amino acid Q (Glutamine). It is clear however that this numbering will be different for amino acids after position 53 (Threonine) for each of the EPOshort1 polypeptides and will depend on the number of amino acids that are missing in EPOshort1 compared to EPOwt. The corresponding amino acid(s) that is/are mutated is/are easily identified by substracting the number of amino acids that are missing in the specific EPOshort1 peptide compared to EPOwt, to the position number of the amino acid in SEQ ID NO: 3 (e.g. if the polypeptide EPOshort1 lacks the amino acids 54 to 82 (29 amino acids), the G104S mutation would correspond to G75S for this specific polypeptide)).

In another embodiment, the present invention resides in analogs of EPO polypeptides corresponding to an isolated polypeptide comprising or consisting of an EPOshort1 or a variant of the EPOshort1 polypeptides described hereabove, which differ in addition from such polypeptides such as having from 1 to 6 additional sites for glycosylation. Glycosylation of a protein, with one or more oligosaccharide groups, occurs at specific locations along a polypeptide backbone and affects the physical properties of the protein such as protein stability, secretion, subcellular localisation, and biological activity. Glycosylation is usually of two types: O-linked oligosaccharides are attached to serine or threonine residues and N-linked oligosaccharides are attached to asparagine residues. One type of oligosaccharide found on both N-linked and 0-linked oligosaccharides is N-acetylneuraminic acid (sialic acid), which is a family of amino sugars containing 9 or more carbon atoms. Sialic acid is usually the terminal residue on both N-linked and 0-linked oligosaccharides and, because it bears a negative charge, confers acidic properties to the glycoprotein. Two N-linked glycosylation sites: asparagine residues at positions 24 and 83 of EPOwtm and the 0-linked glycosylation site: serine residue located at position 126 of EPOwtm are retained in EPOv1m (while the N-linked glycosylation site at position 38 (asparagine residue) of EPOwtm is not present in EPOv1m). The polypeptides of the present invention include analogs of EPOv1 or of EPOshort1 or of a variant of the EPOshort1 polypeptides described hereabove with one or more changes in the amino acid sequence which result in an increase in the number of sites for sialic acid attachment. These glycoprotein analogs may be generated by site-directed mutagenesis having additions, deletions, or substitutions of amino acid residues that increase or alter sites that are available for glycosylation. EPO analogs of the present invention having levels of sialic acid greater than those found in human erythropoietin are generated by adding glycosylation sites which do not perturb the secondary or tertiary conformation required for biological activity. The polypeptides of the present invention also include EPO analogs having increased levels of carbohydrate attachment at a glycosylation site which usually involve the substitution of one or more amino acids in close proximity to an N-linked or 0-linked site. The polypeptides of the present invention also include EPO analogs having one or more amino acids extending from the carboxy terminal end of erythropoietin and providing at least one additional carbohydrate site. The polypeptides of the present invention also include EPO analogs having an amino acid sequence which includes a rearrangement of at least one site for glycosylation. Such a rearrangement of glycosylation site involves the deletion of one or more glycosylation sites in EPOv1 or in EPOshort1 or in a variant of the EPOshort1 polypeptides described hereabove and the addition of one or more non-naturally occurring glycosylation sites. Increasing the number of carbohydrate chains on erythropoietin, and therefore the number of sialic acids per erythropoietin molecules may confer advantageous properties such as increased solubility, greater resistance to proteolysis, reduced immunogenecity, increased serum half-life, and increased biological activity. Erythropoietin analogs with additional glycosylation sites are disclosed in more detail in European Patent Application 640 619, PCT application WO0024893 and WO0181405.

In a preferred embodiment, such EPO analogs of the present invention comprise or consist of the EPOshort1 polypeptide or of a variant of the EPOshort1 polypeptides described hereabove, which includes at least one additional N-linked glycosylation site at position 84, 96, 113, 115, 116 or 141. As already explained hereabove, the position given is relative to the numbering of the amino acids at SEQ ID NO: 3. This numbering will be different for amino acids after position 53 (Threonine) for each of the EPOshort1 or variant polypeptides and will depend on the number of amino acids that are missing in EPOshort1, or in the variant of the EPOshort1 polypeptides, compared to EPOwt. In another embodiment, such EPO analogs includes at least two additional glycosylation sites, or at least three additional glycosylation sites, or at least four additional glycosylation sites.

In a preferred embodiment, these EPO analogs of the present invention comprise or consist of the EPOshort1 polypeptide or of a variant of the EPOshort1 polypeptides described hereabove, modified by a modification selected from the following:

G84N and Q86T;

L96N;

L96N and S98T;

A95S, L96N and S98T;

Q113N, P114V and W115T;

P114V, W115N and P117T;

P114V, W115N and P117S;

P114A, W115N and P117T;

P114S, W115N and P117T;

P114S W115N, E116G and P117T;

P114V, W115N, E116G and P117T;

P114S W115N, P117T and Q119T;

N110Q, P114S, W115N and P117T;

P114S W115N, P117T, R189A;

L96N, S98T, P114S, W115N and P117T;

E116N, P117I and L118T;

P114S, E116N, P117I and L118T;

A141N;

A141N and K143T;

P114V, W115N, P117T, A141N and K143T;

A151P and A152T;

A152T;

A152N and A154S;

D163N and F165T;

F165N and K167T.

As already mentioned hereabove, the position given is relative to the numbering of the amino acids at SEQ ID NO: 3. This numbering will be different for amino acids after position 53 (Threonine) for each of the EPOshort1 polypeptides and will depend on the number of amino acids that are missing in EPOshort1, or in the variant of the EPOshort1 polypeptides, compared to EPOwt.

In a further preferred embodiment, the peptides described here above are mature peptide lacking the N-terminal signal peptide. More particularly, the polypeptides of the present invention lack the signal peptide consisting of amino acids 1 to 27 of SEQ ID NO: 3. Therefore, in a particular aspect, the invention resides in a polypeptide comprising or consisting of an EPOshort1 polypeptide, or variants or analogs of said polypeptides as disclosed here above, lacking amino acids 1 to 27. In yet another particular aspect, the invention resides in a polypeptide comprising or consisting of the sequence of amino acids 28 to 164 of SEQ ID NO: 4 or variants or analogs of said sequence as defined hereabove.

In a further aspect, the present invention resides in an isolated polypeptide comprising or consisting of a homolog of an EPOshort1 polypeptide, or a variant of said EPOshort1 polypeptide or an analog of EPO polypeptides described here above in this section 1.2. In a particular embodiment, said homolog is defined as an active polypeptide having at least 80% amino acid sequence identity with the EPOshort1 polypeptide, or the variant of said EPOshort1 polypeptide or the analog of EPO polypeptides. Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide is from 180 to 136 amino acids in length, from 170 to 136 amino acids in length, often from 160 to 136 amino acids in length, more often 164 amino acids in length, more often 140 amino acids in length, more often 139 amino acids in length, more often 138 amino acids in length, more often 137 amino acids in length, more often 136 amino acids in length. In a particular embodiment, said homolog consists or comprises an active polypeptide having at least 80% amino acid sequence identity with the polypeptide set forth at SEQ ID NO: 4 (EPOv1) or the polypeptide having the sequence of amino acids 28 to 164 of SEQ ID NO: 4 (EPOv1m). Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide of EPOv1 is 180 amino acids in length, more often 170 amino acids in length, more often 166 amino acids in length, more often amino acids in length, more often from 165 amino acids in length, more often 164 amino acids in length. Ordinarily, the homolog polypeptide of EPOv1m is 150 amino acids in length, more often 140 amino acids in length, more often 139 amino acids in length, more often 138 amino acids in length, more often from 137 amino acids in length, more often 136 amino acids in length.

“Percent (%) amino acid sequence identity” with respect to the EPO polypeptide sequences identified herein is defined as the percentage of amino acid residues in a candidate sequence that are identical with the amino acid residues in the specific EPO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol Biol. (1990). 215 (3): 403-410). Those skilled in the art can determine appropriate parameters for measuring alignment, including any algorithms needed to achieve maximal alignment over the full length of the sequences being compared.

1.3 EPOshort2 Polypepltides and Variants Thereof:

In a further aspect, the invention resides in an isolated polypeptide consisting of an EPO polypeptide differing from the sequence set forth at SEQ ID NO: 3 by the lack of at least one, preferably at least two, more preferably at least three, even more preferably at least four, even more preferably at least five, even more preferably at least six, even more preferably at least seven, even more preferably at least eight, even more preferably at least nine, even more preferably at least ten, even more preferably at least eleven, even more preferably at least twelve, even more preferably at least thirteen, even more preferably at least fourteen, even more preferably at least fifteen, even more preferably at least sixteen, even more preferably at least seventeen, even more preferably at least eighteen, even more preferably at least nineteen, even more preferably at least twenty, even more preferably at least twenty-one, even more preferably at least twenty-two, even more preferably at least twenty-three, even more preferably at least twenty-four, even more preferably at least twenty-five, even more preferably at least twenty-six, even more preferably at least twenty-seven, even more preferably at least twenty-eight, even more preferably twenty-nine, even more preferably thirty, even more preferably thirty-one, even more preferably thirty-two, even more preferably thirty-three, even more preferably thirty-four, even more preferably thirty-five, even more preferably thirty-six, even more preferably thirty-seven, even more preferably thirty-eight, even more preferably thirty-nine, even more preferably forty, even more preferably forty-one, even more preferably forty-two, even more preferably forty-three, even more preferably forty-four, even more preferably forty-five, even more preferably forty-six, even more preferably forty-seven, even more preferably forty-eight, even more preferably forty-nine, even more preferably fifty, even more preferably fifty-one, even more preferably fifty-two, even more preferably fifty-three, even more preferably fifty-four, even more preferably fifty-five, even more preferably fifty-six, even more preferably fifty-seven, even more preferably fifty-eight, even more preferably fifty-nine, even more preferably sixty, even more preferably sixty-one, even more preferably sixty-two, even more preferably sixty-three, even more preferably sixty-four, even more preferably sixty-five, even more preferably sixty-six, even more preferably sixty-seven, even more preferably sixty-eight, even more preferably sixty-nine, even more preferably seventy, even more preferably seventy-one, even more preferably seventy-two, even more preferably seventy-three, even more preferably seventy-four, even more preferably seventy-five, even more preferably seventy-six, even more preferably seventy-seven, even more preferably seventy-eight, even more preferably seventy-nine, even more preferably eighty, even more preferably eighty-one, even more preferably eighty-two, even more preferably eighty-three, even more preferably eighty-four, even more preferably eighty-five, even more preferably eighty-six, even more preferably eighty-seven, even more preferably eighty-eight and most preferably eighty-nine amino acids of the amino acids 54 (Threonine) to 142 (Glutamine) of SEQ ID NO: 3. These EPO polypeptides will be named “EPOshort2” here after. In another aspect, the invention resides in an isolated polypeptide comprising an EPOshort2 polypeptide.

The term “isolated” when used to describe the various polypeptides disclosed herein, means polypeptide that has been identified and separated and/or recovered from a component of its natural environment. Isolated products of this invention may thus be contained in a culture supernatant, partially enriched or purified, produced from heterologous sources, cloned in a vector or formulated with a vehicle, etc.

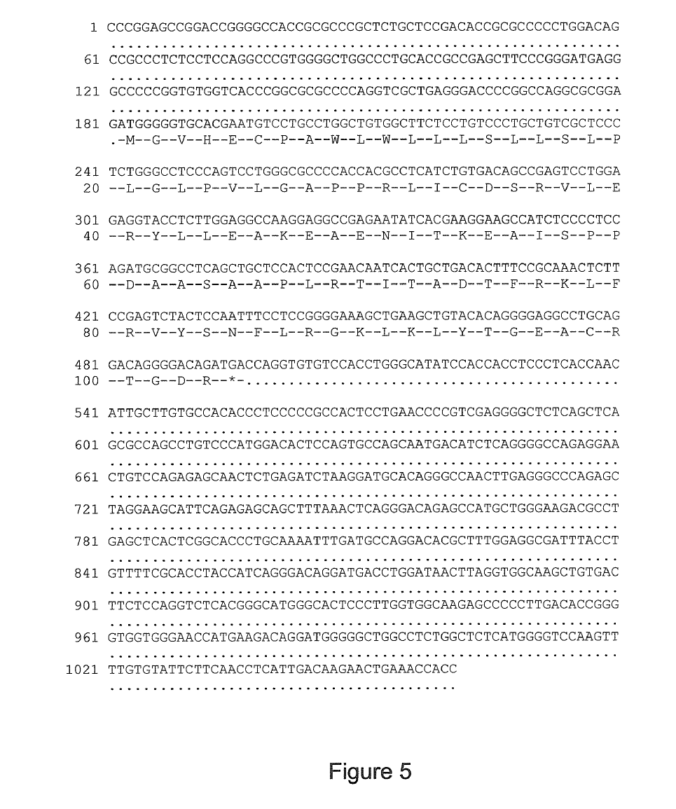

In a preferred embodiment, a polypeptide according to the present invention have the sequence set forth at SEQ ID NO: 6 (named here after EPOv2) and corresponds to a novel transcriptional variant of EPO. EPOv2 is encoded by exons 1, 2 and 5 of the human gene EPO and lacks the 29 amino acids encoded by exon 3 and the 60 amino acids encoded by exon 4 (see FIG. 5). This transcriptional variant is therefore 104 amino acids long in its immature form. The N-terminal signal peptide includes the first 27 amino acids. Once the signal peptide is cleaved, the resulting protein is 77 amino acids long and is named hereafter EPOv2m (amino acid 28 to 104 of SEQ ID NO: 6). As already mentioned hereabove, the removal of the carboxyl-terminal residue has been described for EPOwtm such that the protein expressed by the cells is a 165 amino acid long protein (amino acid 28 to 192 of SEQ ID NO: 3). Therefore, the mature EPOv2m protein might be processed in the same manner by the cell and the resulting protein might be 76 amino acids long (amino acid 28 to 103 of SEQ ID NO: 6). More generally, in an embodiment of the present invention, the EPOshort2 polypeptides described hereabove lacks this carboxyl-terminal Arginine residue.

As described here above, EPOv2 (presented at FIG. 5 and SEQ ID NO: 6) lacks amino acids 54 to 142 of EPOwt which are encoded by exons 3 and 4. It can be concluded that EPOv2 retains the antiparallel long helices αA (residues 8-26 of EPOwtm) and αD helix (residues 138-161 of EPOwtm) but the antiparallel long helices αB (residues 55-83 of EPOwtm) and αC (residues 90-112 of EPOwtm) are absent of EPOv2m. The antiparallel β-sheet: β2 (residues 133-135 of EPOwtm) is also present, but the antiparallel β-sheet: β1 (residues 39-41 of EPOwtm) is absent of EPOv2m. The disulphide bond, Cys 29 to Cys 33 is absent. A large part of the mini-helix αC′ (residues 114-121 of EPOwtm) is retained in EPOv2m but the short helice αB′ helix (residues 47-52 of EPOwtm) is absent. The disulphide bond, Cys 7 to Cys 161, which links together αA helix and and αD should also be retained.

In a further aspect, the present invention resides in an isolated polypeptide comprising or consisting of a variant of the EPOshort2 polypeptides described hereabove. A variant being defined as polypeptides comprising one or several amino acid substitutions as compared to the EPOshort2 polypeptides described hereabove, typically from 0 to 10 amino acid substitutions, even more typically from 0 to 5, 4, 3, 2 or 1 amino acid substitutions. More particularly, the variant polypeptide differs from the EPOshort2 polypeptides described hereabove by at least one, two, three, four, five, six, seven, eight, nine or ten mutations chosen in the group consisting of: E40Q, Q85QQ, G104S, L129G, L129P, L129S, S131N, L132F, SL131-132NF, T134D, G140R and S147C. In a preferred embodiment, the variant polypeptide differs from the sequence of the EPOshort2 polypeptides described hereabove by one or two mutation chosen in the group consisting of: G104S and S147C. In another embodiment, the variant of the EPOshort2 polypeptides differs from the EPOshort2 polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A. In still another embodiment, the variant of the EPOshort2 polypeptides differs from the EPOshort2 polypeptides described hereabove by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A. In still another embodiment, a variant of the EPOshort2 polypeptides differs from the EPOshort2 polypeptides described hereabove by one of the combination mutations chosen in the group consisting of: K72D/S 127E, A57N/H59T, K72D/R177E, R130E/L135S, K79A/K167A, K72A/K79A/K167A, K124A/K179A, K72A/K124A/K179A, K72A/K79A/K124A/K179A, K72A/K79A/K124A/K167A/K179A, K72A/K79A/K124A/K167A/K179A/K181A, N51K/N65K/N110K, and Y42A/N51K. In a preferred embodiment, a variant of the EPOshort2 polypeptides differs from the sequence of the EPOshort2 polypeptides described hereabove by the mutation consisting of C34S. The notation used herein for modification of amino acid sequence means that the wild-type amino acid at the indicated position is changed to the amino acid that immediately follows the respective number. The numbering given is relative to the numbering of the amino acids at SEQ ID NO: 3. Thus for example, the E40Q mutation corresponds to a mutation of the amino acid E (Glutamic acid) at position 40 of SEQ ID NO: 3 into an amino acid Q (Glutamine). It is clear however that this numbering will be different for amino acids after position 53 (Threonine) for each of the EPOshort2 polypeptides and will depend on the number of amino acids that are missing in EPOshort2 compared to EPOwt. The corresponding amino acid(s) that is/are mutated is/are easily identified by substracting the number of amino acids that are missing in the specific EPOshort2 peptide compared to EPOwt, to the position number of the amino acid in SEQ ID NO: 3 (e.g. if the polypeptide EPOshort2 lacks the amino acids 54 to 142 (89 amino acids), the S147C mutation would correspond to S58C for this specific polypeptide).

In another embodiment, the present invention resides in analogs of EPO polypeptides corresponding to an isolated polypeptide comprising or consisting of an EPOshort2 or a variant of the EPOshort2 polypeptides described hereabove, which differ in addition from such polypeptides such as having from 1 to 6 additional sites for glycosylation. Glycosylation of a protein, with one or more oligosaccharide groups, occurs at specific locations along a polypeptide backbone and affects the physical properties of the protein such as protein stability, secretion, subcellular localisation, and biological activity. Glycosylation is usually of two types: O-linked oligosaccharides are attached to serine or threonine residues and N-linked oligosaccharides are attached to asparagine residues. One type of oligosaccharide found on both N-linked and 0-linked oligosaccharides is N-acetylneuraminic acid (sialic acid), which is a family of amino sugars containing 9 or more carbon atoms. Sialic acid is usually the terminal residue on both N-linked and 0-linked oligosaccharides and, because it bears a negative charge, confers acidic properties to the glycoprotein. One N-linked glycosylation site: asparagine residue at position 24 of EPOwtm and the 0-linked glycosylation site: serine residue located at position 126 of EPOwtm are retained in EPOv2m (while the N-linked glycosylation sites at position 38 and 83 (asparagine residues) of EPOwtm are not present in EPOv2m). The polypeptides of the present invention include analogs of EPOv2 or of EPOshort2 or of a variant of the EPOshort2 polypeptides described hereabove with one or more changes in the amino acid sequence which result in an increase in the number of sites for sialic acid attachment. These glycoprotein analogs may be generated by site-directed mutagenesis having additions, deletions, or substitutions of amino acid residues that increase or alter sites that are available for glycosylation. EPO analogs of the present invention having levels of sialic acid greater than those found in human erythropoietin are generated by adding glycosylation sites which do not perturb the secondary or tertiary conformation required for biological activity. The polypeptides of the present invention also include EPO analogs having increased levels of carbohydrate attachment at a glycosylation site which usually involve the substitution of one or more amino acids in close proximity to an N-linked or 0-linked site. The polypeptides of the present invention also include EPO analogs having one or more amino acids extending from the carboxy terminal end of erythropoietin and providing at least one additional carbohydrate site. The polypeptides of the present invention also include EPO analogs having an amino acid sequence which includes a rearrangement of at least one site for glycosylation. Such a rearrangement of glycosylation site involves the deletion of one or more glycosylation sites in EPOv2 or in EPOshort2 or in a variant of the EPOshort2 polypeptides described hereabove and the addition of one or more non-naturally occurring glycosylation sites. Increasing the number of carbohydrate chains on erythropoietin, and therefore the number of sialic acids per erythropoietin molecules may confer advantageous properties such as increased solubility, greater resistance to proteolysis, reduced immunogenecity, increased serum half-life, and increased biological activity. Erythropoietin analogs with additional glycosylation sites are disclosed in more detail in European Patent Application 640 619, PCT application WO0024893 and WO0181405.

In a preferred embodiment, such EPO analogs of the present invention comprise or consist of the EPOshort2 polypeptide or of a variant of the EPOshort2 polypeptides described hereabove, which includes at least one additional N-linked glycosylation site at position 84, 96, 113, 115, 116 or 141. As already explained hereabove, the position given is relative to the numbering of the amino acids at SEQ ID NO: 3. This numbering will be different for amino acids after position 53 (Threonine) for each of the EPOshort2 or variant polypeptides and will depend on the number of amino acids that are missing in EPOshort2, or in the variant of the EPOshort2 polypeptides, compared to EPOwt. In another embodiment, such EPO analogs includes at least two additional glycosylation sites, or at least three additional glycosylation sites, or at least four additional glycosylation sites.

In a preferred embodiment, these EPO analogs of the present invention comprise or consist of the EPOshort2 polypeptide or of a variant of the EPOshort2 polypeptides described hereabove, modified by a modification selected from the following:

G84N and Q86T;

L96N;

L96N and S98T;

A95S, L96N and S98T;

Q113N, P114V and W115T;

P114V, W115N and P117T;

P114V, W115N and P117S;

P114A, W115N and P117T;

P114S, W115N and P117T;

P114S W115N, E116G and P117T;

P114V, W115N, E116G and P117T;

P114S W115N, P117T and Q119T;

N110Q, P114S, W115N and P117T;

P114S W115N, P117T, R189A;

L96N, S98T, P114S, W115N and P117T;

E116N, P1171 and L118T;

P114S, E116N, P117I and L118T;

A141N;

A141N and K143T;

P114V, W115N, P117T, A141N and K143T;

A151P and A152T;

A152T;

A152N and A154S;

D163N and F165T;

F165N and K167T.

As already mentioned hereabove, the position given is relative to the numbering of the amino acids at SEQ ID NO: 3. This numbering will be different for amino acids after position 53 (Threonine) for each of the EPOshort2 polypeptides and will depend on the number of amino acids that are missing in EPOshort2, or in the variant of the EPOshort2 polypeptides, compared to EPOwt.

In a further preferred embodiment, the peptides described here above are mature peptide lacking the N-terminal signal peptide. More particularly, the polypeptides of the present invention lack the signal peptide consisting of amino acids 1 to 27 of SEQ ID NO: 3. Therefore, in a particular aspect, the invention resides in a polypeptide comprising or consisting of an EPOshort2 polypeptide or a variant or an analog of said polypeptide as disclosed here above, lacking amino acids 1 to 27. In yet another particular aspect, the invention resides in a polypeptide comprising or consisting of the sequence of amino acids 28 to 104 of SEQ ID NO: 6 or variants or analogs of said sequence as defined hereabove.

In a further aspect, the present invention resides in an isolated polypeptide comprising or consisting of a homolog of an EPOshort2 polypeptide, or a variant of said EPOshort2 polypeptide or an analog of EPO polypeptides described here above in this section 1.3. In a particular embodiment, said homolog is defined as an active polypeptide having at least 80% amino acid sequence identity with the EPOshort2 polypeptide, or the variant of said EPOshort2 polypeptide or the analog of EPO polypeptides. Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide is from 120 to 77 amino acids in length, from 110 to 77 amino acids in length, often from 105 to 77 amino acids in length, more often 104 amino acids in length, more often 80 amino acids in length, more often 79 amino acids in length, more often 78 amino acids in length, more often 77 amino acids in length. In a particular embodiment, said homolog consists in or comprises an active polypeptide having at least 80% amino acid sequence identity with the polypeptide set forth at SEQ ID NO: 6 (EPOv2) or the polypeptide having the sequence of amino acids 28 to 104 of SEQ ID NO: 6 (EPOv2m). Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide of EPOv2 is 125 amino acids in length, more often 115 amino acids in length, more often 110 amino acids in length, more often amino acids in length, more often from 109 amino acids in length, more often from 108 amino acids in length, more often from 107 amino acids in length, more often from 106 amino acids in length, more often from 105 amino acids in length, more often 104 amino acids in length. Ordinarily, the homolog polypeptide of EPOv2m is 90 amino acids in length, more often 85 amino acids in length, more often 80 amino acids in length, more often 79 amino acids in length, more often 78 amino acids in length, more often 77 amino acids in length.

“Percent (%) amino acid sequence identity” with respect to the EPO polypeptide sequences identified herein is defined as the percentage of amino acid residues in a candidate sequence that are identical with the amino acid residues in the specific EPO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol Biol. (1990). 215 (3): 403-410). Those skilled in the art can determine appropriate parameters for measuring alignment, including any algorithms needed to achieve maximal alignment over the full length of the sequences being compared.

1.4 EPOv3 Polypeptides and Variants Thereof:

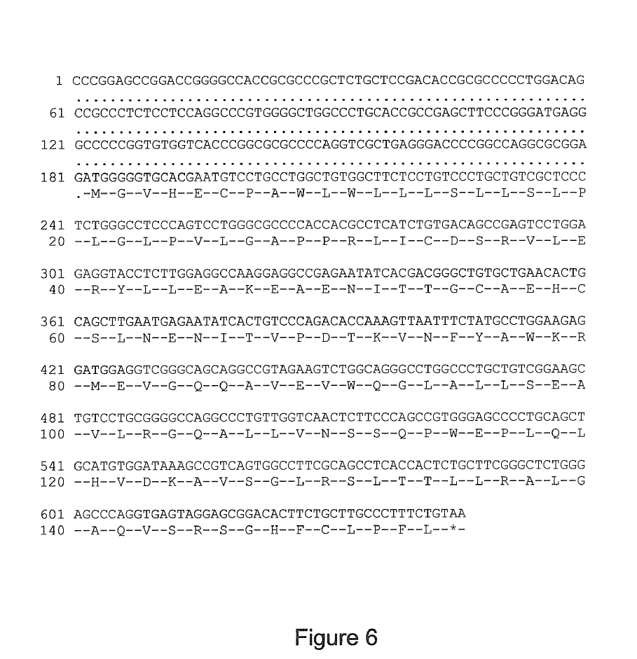

In a further aspect, the invention resides in an isolated polypeptide comprising or consisting of the sequence set forth at SEQ ID NO: 8. The polypeptide having the sequence set forth at SEQ ID NO: 8 corresponds to the C-terminal part of the novel transcriptional variant of EPO disclosed here for the first time and is encoded by 3′ end of the exon 4A. Said exon 4A is longer at the 3′ end as compared to exon 4 which encode for the wild-type EPO (see FIGS. 3 and 6).

The term “isolated” when used to describe the various polypeptides disclosed herein, means polypeptide that has been identified and separated and/or recovered from a component of its natural environment. Isolated products of this invention may thus be contained in a culture supernatant, partially enriched or purified, produced from heterologous sources, cloned in a vector or formulated with a vehicle, etc.

In a further aspect, the present invention resides in an isolated polypeptide comprising or consisting of a variant of the polypeptide set forth at SEQ ID NO: 8. A variant being defined as a polypeptide having at least 75% amino acid sequence identity with the sequence SEQ ID NO: 8, preferably at least 80% amino acid sequence identity, more preferably at least 90% amino acid sequence identity, more preferably at least 95% amino acid sequence identity, more preferably at least about 98% amino acid sequence identity and most preferably at least 99% amino acid sequence identity. Ordinarily, the variant polypeptides are at least 8 amino acids in length, often at least 10 amino acids in length, more often at least 12 amino acids in length. More preferably, the variant are deferring from SEQ ID NO: 8 by two and even more preferably by one amino acid.

“Percent (%) amino acid sequence identity” with respect to the EPO polypeptide sequences identified herein is defined as the percentage of amino acid residues in a candidate sequence that are identical with the amino acid residues in the specific EPO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol Biol. (1990). 215 (3): 403-410). Those skilled in the art can determine appropriate parameters for measuring alignment, including any algorithms needed to achieve maximal alignment over the full length of the sequences being compared.

In a further aspect, the invention resides in an isolated polypeptide comprising or consisting of the sequence set forth at SEQ ID NO: 9. The polypeptide of SEQ ID NO: 9 (named hereafter EPOv3) is a novel transcriptional variant of EPO, which is encoded by exons 1, 2, 3 and a longer exon 4 (named herein exon 4A) of the human gene EPO (see FIG. 6). This longer exon 4 (exon 4A) is coding for the C-terminal part of the novel transcriptional variant of EPO and is disclosed here for the first time. Said exon 4A is longer at the 3′ end as compared to exon 4 which encode for the wild-type EPO (see FIGS. 3 and 6).

In FIG. 6, nucleotides 1 to 607 of the human transcriptional variant presented represents the junction of exons 1, 2, 3 and the 5′ end of exon 4A and nucleotides 608 to 647 represents the 3′ end of the exon 4A. EPOv3 is a polypeptide of 154 amino acid in its immature form. The N-terminal signal peptide includes the first 27 amino acids. Once the signal peptide is cleaved, the resulting protein is 127 amino acids long and is named hereafter EPOv3m (amino acid 28 to 154 of SEQ ID NO: 9). The sequence of EPOv3 is common to the EPOwt (presented at FIG. 3 and SEQ ID NO: 3) from amino acid 1 to 142 and differs in its C-terminus (from amino acid 143 to 154). It can be concluded that EPOv3 (presented in FIG. 4 and SEQ ID NO: 9) retains the antiparallel long helices, αA (residues 8-26 of EPOwtm), αB (residues 55-83 of EPOwtm) and αC (residues 90-112 of EPOwtm). The αD helix (residues 138-161 of EPOwtm) is not present in EPOv3m. The antiparallel β-sheets: β1 (residues 39-41 of EPOwtm) and β2 (residues 133-135 of EPOwtm) are also present. The disulphide bond, Cys 29 to Cys 33, which links the end of the αA helix with part of the AB loop should also be retained. The two additional short helices, αB′ helix (residues 47-52 of EPOwtm) and the mini-helix αC′ (residues 114-121 of EPOwtm) are also retained in EPOv3m. The cysteine Cys 7 is retained but cystein Cys 161 which form a disulphide bridge with Cys 7 is not present. However, the novel 3′ end of exon 4A encodes a cystein at position 150 of SEQ ID NO: 9 (EPOv3), or position 123 in EPOv3m, which may form a disulphide bridge with Cys 7.

In a further aspect, the present invention resides in an isolated polypeptide comprising or consisting of a variant of EPOv3. In a particular embodiment, a variant of the polypeptide set forth at SEQ ID NO: 9 (EPOv3) is defined as an active polypeptide having at least 80% amino acid sequence identity with the sequence SEQ ID NO: 9, preferably at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the EPOv3 variant polypeptides are at least 125 amino acids in length, often at least 140 amino acids in length, often at least 145 amino acids in length, often at least 150 amino acids in length, more often at least 154 amino acids in length. More preferably, the EPOv3 variants of the present invention resides in polypeptide comprising one or several amino acid substitutions as compared to the SEQ ID NO: 9, typically from 0 to 10 amino acid substitutions, even more typically from 0 to 5, 4, 3, 2 or 1 amino acid substitutions. More particularly, the EPOv3 variant polypeptide differs from the sequence set forth at SEQ ID NO: 9 by at least one, two, three, four, five, six, seven, eight, nine or ten mutations chosen in the group consisting of: E40Q, D70N, Q85QQ, G104S, L129G, L129P, L129S, S131N, L132F, T134D, G140R and SL131-132NF. In another embodiment, the EPOv3 variant polypeptide differs from the sequence set forth at SEQ ID NO: 9 by one or two mutation chosen in the group consisting of: D70N and G104S. In yet another embodiment, the EPOv3 variant polypeptide differs from the sequence set forth at SEQ ID NO: 9 by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S. In still another embodiment, the EPOv3 variant polypeptide differs from the sequence set forth at SEQ ID NO: 9 by one of the combination of mutations chosen in the group consisting of: K72D/S127E, A57N/H59T, R130E/L135S, N51K/N65K/N110K, and Y42A/N51K. In a preferred embodiment, the EPOv3 variant polypeptide differs from the sequence set forth at SEQ ID NO: 9 by the mutation consisting of C34S.

In a particular embodiment, the EPOv3 variant polypeptide differs from the polypeptide hereabove described (e.g. from the polypeptide set forth at SEQ ID NO: 9; or from a polypeptide differing from the sequence set forth at SEQ ID NO: 9 by at least one, two, three, four, five, six, seven, eight, nine or ten mutations chosen in the group consisting of: E40Q, D70N, Q85QQ, G104S, L129G, L129P, L129S, S131N, L132F, T134D, G14OR and SL131-132NF; or from a polypeptide differing from SEQ ID NO: 9 by one or two mutation chosen in the group consisting of: D70N and G104S; or from a polypeptide differing from the sequence set forth at SEQ ID NO: 9 by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S; or from a polypeptide differing from the sequence set forth at SEQ ID NO: 9 by one of the combination of mutations chosen in the group consisting of: K72D/S127E, A57N/H59T, R130E/L135S, N51K/N65K/N110K, and Y42A/N51K; or from a polypeptide differing from the sequence set forth at SEQ ID NO: 9 by the mutation consisting of C34S) such as having from 1 to 6 additional sites for glycosylation. Glycosylation of a protein, with one or more oligosaccharide groups, occurs at specific locations along a polypeptide backbone and affects the physical properties of the protein such as protein stability, secretion, subcellular localisation, and biological activity. Glycosylation is usually of two types. O-linked oligosaccharides are attached to serine or threonine residues and N-linked oligosaccharides are attached to asparagine residues. One type of oligosaccharide found on both N-linked and 0-linked oligosaccharides is N-acetylneuraminic acid (sialic acid), which is a family of amino sugars containing 9 or more carbon atoms. Sialic acid is usually the terminal residue on both N-linked and 0-linked oligosaccharides and, because it bears a negative charge, confers acidic properties to the glycoprotein. The N-linked glycosylation sites: asparagine residues at positions 24, 38 and 83 of EPOwtm are retained in EPOv3m (while the 0-linked glycosylation site: serine residue located at position 126 of EPOwtm is not present in EPOv3m). In a particular embodiment, the EPOv3 variants of the present invention include analogs with one or more changes in the amino acid sequence which result in an increase in the number of sites for sialic acid attachment. These glycoprotein analogs may be generated by site-directed mutagenesis having additions, deletions, or substitutions of amino acid residues that increase or alter sites that are available for glycosylation. In a particular embodiment, EPOv3 variants having levels of sialic acid greater than those found in human erythropoietin are generated by adding glycosylation sites which do not perturb the secondary or tertiary conformation required for biological activity. In a particular embodiment, the EPOv3 variants of the present invention also include analogs having increased levels of carbohydrate attachment at a glycosylation site which usually involve the substitution of one or more amino acids in close proximity to an N-linked or 0-linked site. In a particular embodiment, the EPOv3 variants of the present invention also include analogs having one or more amino acids extending from the carboxy terminal end of erythropoietin and providing at least one additional carbohydrate site. In a particular embodiment, the EPOv3 variants of the present invention also include analogs having an amino acid sequence which includes a rearrangement of at least one site for glycosylation. Such a rearrangement of glycosylation site involves the deletion of one or more glycosylation sites in EPOv3 (or in a polypeptide differing from the sequence set forth at SEQ ID NO: 9 by at least one, two, three, four, five, six, seven, eight, nine or ten mutations chosen in the group consisting of: E40Q, D70N, Q85QQ, G104S, L129G, L129P, L129S, S131N, L132F, T134D, G140R and SL131-132NF; or by one or two mutation chosen in the group consisting of: D70N and G104S; or by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S; or by one of the combination of mutations chosen in the group consisting of: K72D/S127E, A57N/H59T, R130E/L135S, N51K/N65K/N110K, and Y42A/N51K; or by the mutation consisting of C34S) and the addition of one or more non-naturally occurring glycosylation sites. Increasing the number of carbohydrate chains on erythropoietin, and therefore the number of sialic acids per erythropoietin molecules may confer advantageous properties such as increased solubility, greater resistance to proteolysis, reduced immunogenecity, increased serum half-life, and increased biological activity. Erythropoietin analogs with additional glycosylation sites are disclosed in more detail in European Patent Application 640 619, PCT application WO0024893 and WO0181405.

In a particular embodiment, EPOv3 variants of the present invention comprise or consist of an amino acid sequence of SEQ ID NO: 9 (or an amino acid sequence differing from such a sequence by at least one, two, three, four, five, six, seven, eight, nine or ten mutations chosen in the group consisting of: E40Q, D70N, Q85QQ, G104S, L129G, L129P, L129S, S131N, L132F, T134D, G140R and SL131-132NF; or by one or two mutation chosen in the group consisting of: D70N and G104S; or by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S; or by one of the combination of mutations chosen in the group consisting of: K72D/S127E, A57N/H59T, R130E/L135S, N51K/N65K/N110K, and Y42A/N51K; or by the mutation consisting of C34S), which includes at least one additional N-linked glycosylation site at position 57, 78, 79, 80, 82, 84, 96, 113, 115, 116 or 141. In another embodiment, such EPOv3 variants includes at least two additional glycosylation sites, or at least three additional glycosylation sites, or at least four additional glycosylation sites.

In a preferred embodiment, the EPOv3 variants of the present invention comprise or consist of the sequence of SEQ ID NO: 9 (or an amino acid sequence differing from SEQ ID NO: 9 by at least one, two, three, four, five, six, seven, eight, nine or ten mutations chosen in the group consisting of: E40Q, D70N, Q85QQ, G104S, L129G, L129P, L129S, S131N, L132F, T134D, G140R and SL131-132NF; or by one or two mutation chosen in the group consisting of: D70N and G104S; or by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S; or by one of the combination of mutations chosen in the group consisting of: K72D/S127E, A57N/H59T, R130E/L135S, N51K/N65K/N110K, and Y42A/N51K; or by the mutation consisting of C34S), modified by a modification selected from the following:

A57N and H59T;

W78N and R80T;

K79N and M81T;

R80N and E82T;

A57N, H59T, R80N and E82T;

E82N and G84T;

G84N and Q86T;

L96N;

L96N and S98T;

A95S, L96N and S98T;

Q113N, P114V and W115T;

P114V, W115N and P117T;

P114V, W115N and P117S;

P114A, W115N and P117T;

P114S, W115N and P117T;

P114S W115N, E116G and P117T;

P114V, W115N, E116G and P117T;

A57N, H59T, R80N, E82N, P114V, W115N and P117T;

N110Q, P114S, W115N and P117T;

P114S W115N, P117T and Q119T;

L96N, S98T, P114S, W115N and P117T;

A57N, H59T, P114V, W115N and P117T;

E116N, P117I and L118T;

P114S, E116N, P117I and L118T;

A141N.

The notation used herein for modification of amino acid sequence means that the wild-type amino acid at the indicated position is changed to the amino acid that immediately follows the respective number. The numbering is relative to the numbering of the amino acids at SEQ ID NO: 9. Thus for example, the A57N mutation corresponds to a mutation of the amino acid A (Alanine) at position 57 of SEQ ID NO: 9 into an amino acid N (Asparagine).

The EPOv3 variant may also be an analog having an amino acid sequence which includes a rearrangement of at least one site for glycosylation. The rearrangement may comprise a deletion of any of the N-linked carbohydrate sites in human erythropoietin and an addition of an N-linked carbohydrate site at position 115 of SEQ ID NO: 9. Preferably, these EPOv3 variants comprise or consist of the sequence of SEQ ID NO: 9 (or an amino acid sequence SEQ ID NO: 9 by at least one, two, three, four, five, six, seven, eight, nine or ten mutations chosen in the group consisting of: E40Q, D70N, Q85QQ, G104S, L129G, L129P, L129S, S131N, L132F, T134D, G140R and SL131-132NF; or by one or two mutation chosen in the group consisting of: D70N and G104S; or by at least one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S; or by one of the combination of mutations chosen in the group consisting of: K72D/S127E, A57N/H59T, R130E/L135S, N51K/N65K/N110K, and Y42A/N51K; or by the mutation consisting of C34S), modified by a modification selected from the following:

N51Q, P114S, W115N and P117T;

N65Q, P114S, W115N and P117T;

N110Q, P114S, W115N and P117T.

In a preferred embodiment, the peptides described here above are mature peptide lacking the N-terminal signal peptide. More particularly, the polypeptides of the present invention lack the signal peptide consisting of amino acids 1 to 27 of SEQ ID NO: 9. Therefore, in a particular aspect, the invention resides in a polypeptide comprising or consisting of an EPOv3 polypeptide or a variant or an analog of said polypeptide as disclosed here above, lacking amino acids 1 to 27. In yet another particular aspect, the invention resides in a polypeptide comprising or consisting of the sequence of amino acids 28 to 154 of SEQ ID NO: 9 or variants of said sequence as defined hereabove.

In a further aspect, the present invention resides in an isolated polypeptide comprising or consisting of a homolog of a polypeptide described here above in this section 1.4. In a particular embodiment, said homolog is defined as an active polypeptide having at least 80% amino acid sequence identity with the polypeptide of reference or the variant of said polypeptide or the analog said polypeptide. Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide is from 170 to 127 amino acids in length, often from 160 to 127 amino acids in length, more often from 154 to 127 amino acids in length, more often 130 amino acids in length, more often 129 amino acids in length, more often 128 amino acids in length, more often 127 amino acids in length. In a particular embodiment, said homolog consists in or comprises an active polypeptide having at least 80% amino acid sequence identity with the polypeptide set forth at SEQ ID NO: 9 (EPOv3) or the polypeptide having the sequence of amino acids 28 to 154 of SEQ ID NO: 9 (EPOv3m). Preferably said identity is at least 90% amino acid sequence identity, more preferably at least 95% amino acid sequence identity, more preferably at least 98% amino acid sequence identity, more preferably at least 99% amino acid sequence identity and most preferably at least 99.5% amino acid sequence identity. Ordinarily, the homolog polypeptide of EPOv3 is 170 amino acids in length, more often 160 amino acids in length, more often 159 amino acids in length, more often from 158 amino acids in length, more often from 157 amino acids in length, more often from 156 amino acids in length, more often from 1155 amino acids in length, more often from 154 amino acids in length. Ordinarily, the homolog polypeptide of EPOv3m is 150 amino acids in length, more often 140 amino acids in length, more often 130 amino acids in length, more often 129 amino acids in length, more often 128 amino acids in length, more often 127 amino acids in length.

“Percent (%) amino acid sequence identity” with respect to the EPO polypeptide sequences identified herein is defined as the percentage of amino acid residues in a candidate sequence that are identical with the amino acid residues in the specific EPO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. J Mol Biol. (1990). 215 (3): 403-410). Those skilled in the art can determine appropriate parameters for measuring alignment, including any algorithms needed to achieve maximal alignment over the full length of the sequences being compared.

1.5 Biological Activity of the EPO Polypeptides and Variants of the Present Invention:

Preferably any of the above or below described EPO polypeptides, variants and analogs retain at least some biological activity. More preferably said biological activity is at least one of the following: binding to EPOR, induction of tyrosine phosphorylation of JAK2 in EPOR expressing cells, stimulation of proliferation of EPOR expressing cells, stimulating red blood cell production in a mammal in particular in human, induction of proliferation and/or terminal maturation of erythroid cells of mammalian origin in particular of human origin in vitro and/or in vivo, vasoactive action (vasoconstriction or vasodilatation) in particular induction of hypertension in a mammal (in particular in human), increasing hematocrit in a mammal in particular in human, hyperactivating platelets, pro-coagulant activity, increasing production of thrombocytes, neuroprotective activity in vitro and/or in vivo in a mammal in particular in human, neurotrophic activity in vitro and/or in vivo in a mammal in particular in human, enhancement of the survival of neuronal cells in vitro and/or in vivo in a mammal in particular in human, cardioprotective activity in vitro and/or in vivo in a mammal in particular in human, enhancement of the survival of cardiomyocyte cells in vitro and/or in vivo in a mammal in particular in human, stimulation of the proliferation of cancer cells of mammalian origin in particular of human origin (preferably of cells originating from the kidney, prostate, ovary or breast, or of lymphoma cells (in particular follicular lymphomas, cutaneous T cell lymphomas, Hodgkin's or non-Hodgkin's lymphomas), or of leukaemia cells (in particular chronic lymphocytic leukemia or chronic myeloid leukaemia), or of multiple myeloma cells, or of cells of tumors affecting the Central Nervous System (in particular glioblastoma or neuroblastoma)) in vitro and/or in vivo, in vitro and/or in vivo antiviral activity (in particular against hepatitis B or C or HIV).

Even more preferably said biological activity is at least one of the following: neuroprotective activity in vitro and/or in vivo in a mammal in particular in human, neurotrophic activity in vitro and/or in vivo in a mammal in particular in human, enhancement of the survival of neuronal cells in vitro and/or in vivo in a mammal in particular in human, cardioprotective activity in vitro and/or in vivo in a mammal in particular in human, enhancement of the survival of cardiomyocyte cells in vitro and/or in vivo in a mammal in particular in human.

These biological activities can be verified using several biological assays that are known per se in the art (non-limiting examples of such assays are described in the example part here below). More preferably any of the above or below described EPO polypeptides, variants and analogs retain at least (or at least about) 50, 60, 70, 75, 80, 85, 90, 95, 96, 97, 98, 99, or 100% of the biological activity as compared to the biological activity of the native/unmodified/wild-type EPO protein (an unmature form of this protein is given at SEQ ID NO: 3). In some embodiments of this aspect of the invention, the protein can have higher biological activity than the native/unmodified, wild-type protein.

In an embodiment of the present invention, the above or below described EPO polypeptides, variants and analogs have a tissue protective activity in mammal in particular in human, without substantially increasing hematocrit level in said mammal. The term “tissue protective” refers to the defense of a tissue against the effects of cellular damage that are typically associated with the experience by a tissue or organ of ischemia/hypoxia, trauma, toxicity and/or inflammation. Cellular damage may lead to apoptosis and/or necrosis (i.e., toxic cell death). Thus, a “tissue protective” effect guards a tissue from experiencing the degree of apoptosis and/or toxic cell death normally associated with a given traumatic, inflammatory, toxic or ischemic injury. For example, EPO has been found to be neuroprotective (Siren A L, et al., (Proc Natl Acad Sci USA. 2001, 98(7):4044-9) and Brines M L et al., (Proc Natl Acad Sci USA. 2000; 97(19):10526-31)) and cardioprotective (Parsa C J et al. (J Clin Invest. 2003. 112(7):999-1007, Moon C et al. (Proc Natl Acad Sci USA. 2003;100(20):11612-7) and Calvillo L, et al., (Proc Natl Acad Sci USA. 2003; 100(8):4802-6)). Thus, under such conditions EPO provides a “tissue protective” effect by effectively reducing the necrosis and/or apoptosis normally associated with the ischemic injury (e.g., ischemic stroke). “Tissue protective” also refers to the defense of a tissue against the effects of cellular damage and the ensuing cell death associated with degenerative diseases such as retinopathy, or neurodegenerative disease.

In a preferred aspect of the present invention, the above or below described EPO polypeptides, variants and analogs have tissue protective activity without substantially increasing hematocrit level in said mammal said tissue protective activity being at least one of the following: neuroprotective activity, neurotrophic activity, cardioprotective activity, hepatoprotective activity, protection of the retina, protection of muscle, protection of the lung, protection of the kidney, protection of the small intestine, protection of the adrenal cortex, protection of the adrenal medulla, protection of the capillary endothelia, protection of the testis, protection of the ovary, and protection of the endometrial tissue. In a further preferred aspect of the present invention, the above or below described EPO polypeptides, variants and analogs have tissue protective activity without substantially increasing hematocrit level in said mammal said tissue protective activity being at least one of the following: neuroprotective activity, neurotrophic activity, cardioprotective activity. In a further preferred aspect of the present invention, the above or below described EPO polypeptides, variants and analogs have tissue protective activity without substantially increasing hematocrit level in said mammal said tissue protective activity being neuroprotective activity and/or neurotrophic activity. These activities can be tested using several biological assays that are known per se in the art (non-limiting examples of such assays are described in the example part here below).

In an embodiment of the present invention the above or below described EPO polypeptides, variants and analogs have neuroprotective activity in a mammal, in particular in human. Said neuroprotective activity can be measured using biological tests, in particular the biological test (sciatic nerve crush) described in example 11. Therefore in an embodiment of the present invention, the above or below described EPO polypeptides, variants and analogs induce a reduction of the compound muscle action potential (CMAP) latency of at least about 0.02 ms, preferably at least about 0.05 ms, preferably at least about 0.1 ms, even preferably preferably at least about 0.15 ms following nerve crush. In an embodiment of the present invention, the above or below described EPO polypeptides, variants and analogs induce a reduction of the compound muscle action potential (CMAP) latency of at least about 0.02 ms, preferably at least about 0.05 ms, preferably at least about 0.1 ms, even preferably preferably at least about 0.15 ms at day 7 and compared to non-treated animals, as measured using the biological test described in example 11 (sciatic nerve crush experiment). In an embodiment of the present invention, the above or below described EPO polypeptides, variants and analogs induce a reduction of the compound muscle action potential (CMAP) latency of at least about 0.02 ms, preferably at least about 0.05 ms, preferably at least about 0.1 ms, even preferably preferably at least about 0.15 ms at day 14 and compared to non treated animals, as measured using the biological test described in example 11 (sciatic nerve crush experiment).

In an embodiment of the present invention the above or below described EPO polypeptides, variants and analogs have at least one of the following biological activity: stimulation of mammalian Schwann cells proliferation (in particular of human Schwann cells), stimulation of axonal regeneration in mammals (in particular in human), decrease in TNF alpha expression by mammalian Schwann cells (in particular decrease in TNF alpha produced by human Schwann cells following nerve damage), stimulation of expression of Myelin Basic Protein (MBP) in particular in mammalian Oligodendrocytes and/or mammalian Schwann cells (in particular in human Oligodendrocytes and/or human Schwann cells).

In an embodiment of the present invention the above or below described EPO polypeptides, variants and analogs stimulate the production of Myelin Basic Protein (MBP). Said stimulation of production of Myelin Basic Protein (MBP) can be measured using biological tests, in particular the biological test described in example 15. Therefore in an embodiment of the present invention, the above or below described EPO polypeptides, variants and analogs induce production by at least about 5%, preferably by least about 7%, even preferably by least about 10% at day 16 as measured using the biological test described in example 15.

In a preferred embodiment of the present invention the above or below described EPO polypeptides, variants and analogs have any of the above or below described biological activity, in particular a tissue protective activity as defined above, but do not substantially increase hematocrit level in mammals in particular in human. In a particular embodiment an above or below described EPO polypeptide, variant or analog does not substantially increase hematocrit level when said polypeptide retain less than 90%, preferably less than 80%, preferably less than 70%, preferably less than 60%, preferably less than 50%, preferably less than 40%, preferably less than 30%, preferably less than 20%, preferably less than 10%, preferably less than 5%, preferably less than 2%, preferably less than 1%, even preferably less than 0.5% of the hematotrophic activity of wild-type EPO (in particular of the commercially available product Eprex). Determining a hematotrophic activity is well within the level of one skilled in the art. Hematotrophic activity of the EPO polypeptides of the invention can be neasured and compared to the hematotrophic activity of wild-type EPO using for example the technique described at example 12.

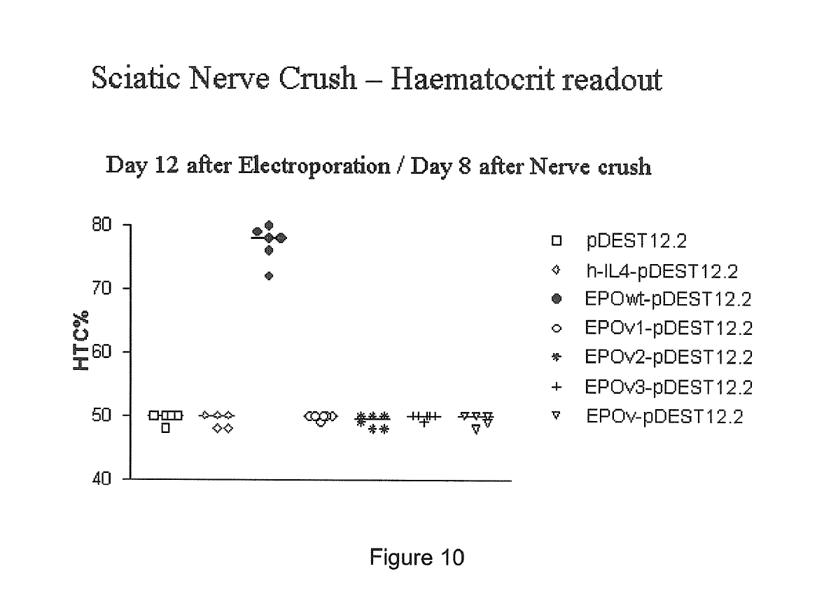

In a particular embodiment an above or below described EPO polypeptide, variant or analog does not subtantially increase hematocrit level when said polypeptide increase hematocrit level by less than about 20%, preferably less than about 15%, preferably less than about 10%, preferably less than about 5%, even preferably less than about 1% as compared to the baseline hematocrit level (i.e. hematocrit level before treatment). Increase in the hematocrit level is measured by comparing the baseline hematocrit (i.e. before treatment) with the hematocrit after treatment. In an embodiment, said treatment is intravenous treatment, three times a week during 8 weeks at the dose of 0.84 μg/kg per injection, in human. In another embodiment the increase in hematocrit level is measured as described at example 12. As shown in FIG. 10, wild type EPO exhibit an increase of hematocrit of about 27% at day 12 in mice (comparaison of the group treated with pDEST 12.2 alone which show an hematocrit level of about 50% to the group treated with EPOwt-pDEST 12.2 which show an hematocrit level of about 77%).

2. Fusion Proteins:

The present invention also relates to fusion proteins comprising a polypeptide as disclosed above (in particular an EPOshort polypeptide or a variant thereof, an EPOshort1 polypeptide or a variant thereof, an EPOshort2 polypeptide or a variant thereof, or an EPOv3 polypeptide or a variant thereof) operably linked to an additional amino acid domain. The additional amino acid domain may be located upstream (N-ter) or downstream (C-ter) from the sequence of the polypeptides described here above. The additional domain may comprise any functional region, providing for instance an increased stability, targeting or bioavailability of the fusion protein; facilitating purification or production, or conferring on the molecule additional biological activity. Specific examples of such additional amino acid sequences include a GST sequence, a His tag sequence, a multimerication domain, the constant region of an immunoglobulin molecule or a heterodimeric protein hormone such as human chorionic gonadotropin (hCG) as described in U.S. Pat. No. 6,193,972. The term “operably linked” indicates that the polypeptide and additional amino acid domain are associated through peptide linkage, either directly or via spacer residues. In this manner, the fusion protein can be produced recombinantly, by direct expression in a host cell of a nucleic acid molecule encoding the same, as will be discussed below. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro.

In a particular embodiment, a fusion protein according to the present invention comprises an immunoglobulin, i.e. the EPO polypeptide, variant or analog of the present invention as disclosed hereabove (in particular an EPOshort polypeptide or a variant thereof, an EPOshort1 polypeptide or a variant thereof, an EPOshort2 polypeptide or a variant thereof, or an EPOv3 polypeptide or a variant thereof) is fused to all or a portion of an immunoglobulin, particularly the Fc portion of a human immunoglobulin. Methods for making immunoglobulin fusion proteins are well known in the art, such as the ones described in WO 01/03737, for example. The person skilled in the art will appreciate that the resulting fusion protein of the invention substantially retains the biological activity of the EPO polypeptide, variant or analog of the present invention. Said biological activity is at least one of the biological activity described here above. In a particular embodiment said biological activity is neuroprotective activity in vitro and/or in vivo in a mammal in particular in human.

The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the EPO polypeptide (in particular the EPOshort polypeptide or a variant thereof, the EPOshort1 polypeptide or a variant thereof, the EPOshort2 polypeptide or a variant thereof, or the EPOv3 polypeptide or a variant thereof) sequence and the immunoglobulin sequence. The amino acid sequence derived from the immunoglobulin may be linked to the C-terminus or to the N-terminus of the EPO polypeptide, variant or analog of the present invention, preferably to the C-terminus. The resulting fusion protein has improved properties, such as an extended residence time in body fluids (half-life), increased specific activity, increased expression level, or the purification of the fusion protein is facilitated.

In a preferred embodiment, the EPO polypeptide, variant or analog of the present invention (in particular an EPOshort polypeptide or a variant thereof, an EPOshort1 polypeptide or a variant thereof, an EPOshort2 polypeptide or a variant thereof, or an EPOv3 polypeptide or a variant thereof) is fused to the constant region of an Ig molecule, e.g. an Fc portion of an Immunoglobulin. Preferably, it is fused to heavy chain regions, like the CH2 and CH3 domains, optionally with the hinge region of human IgG1, for example. The Fc part may e.g. be mutated in order to prevent unwanted activities, such as complement binding, binding to Fc receptors, or the like. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

Further fusion proteins of the EPO polypeptides, variants or analogs of the present invention may be prepared by fusing domains isolated from other proteins allowing the formation or dimers, trimers, etc. Examples for protein sequences allowing the multimerization of the polypeptides of the Invention are domains isolated from proteins such as hCG (WO 97/30161), collagen X (WO 04/33486), C4BP (WO 04/20639), Erb proteins (WO 98/02540), or coiled coil peptides (WO 01/00814).

The present invention also relates to fusion proteins as disclosed herein containing a signal peptide, along with the corresponding DNA sequence encoding such proteins. The signal peptide may be the naturally occurring signal peptide as disclosed herein, or may be a heterologous or synthetic signal peptide.

The present invention also relates to any of the above-disclosed EPO polypeptides, variants or analogs (in particular an EPOshort polypeptide or a variant thereof, an EPOshort1 polypeptide or a variant thereof, an EPOshort2 polypeptide or a variant thereof, or an EPOv3 polypeptide or a variant thereof) comprising an additional N-terminal amino acid residue, preferably a methionine. Indeed, depending on the expression system and conditions, polypeptides of the invention may be expressed in a recombinant host cell with a starting Methionine. This additional amino acid may then be either maintained in the resulting recombinant protein, or eliminated by means of an exopeptidase, such as Methionine Aminopeptidase, according to methods disclosed in the literature (Van Valkenburgh H A and Kahn R A, Methods Enzymol. (2002) 344:186-93; Ben-Bassat A, Bioprocess Technol. (1991) 12:147-59).

3. Preparation of Polypepltides and Fusion Proteins of the Present Invention:

A. Nucleic acid encoding the polypeptides, proteins and fusion proteins of the present invention and vectors:

A further object of the present invention is an isolated nucleic acid molecule encoding the polypeptides, proteins and fusion proteins defined here above. In this regard, the term “nucleic acid molecule” encompasses all different types of nucleic acids, including without limitation deoxyribonucleic acids (e.g., DNA, cDNA, gDNA, synthetic DNA, etc.), ribonucleic acids (e.g., RNA, mRNA, etc.) and peptide nucleic acids (PNA). In a preferred embodiment, the nucleic acid molecule is a DNA molecule, such as a double-stranded DNA molecule or a cDNA molecule. The term “isolated” means nucleic acid molecules that have been identified and separated from at least one contaminant nucleic acid molecule with which it is ordinarily associated in the natural source. An isolated nucleic acid molecule is other than in the form or setting in which it is found in nature. Isolated nucleic acid molecules therefore are distinguished from the specific nucleic acid molecule as it exists in natural cells.

A particular object of this invention resides more specifically in an isolated nucleic acid molecule that comprises or consists of a nucleotide sequence selected from the group consisting of: SEQ ID NO: 5, SEQ ID NO: 7, SEQ ID NO: 10, SEQ ID NO: 11 and SEQ ID NO: 12, or a complementary strand or degenerate sequence thereof, or a nucleic acid coding for the polypeptides of SEQ ID NO: 4, SEQ ID NO: 6, SEQ ID NO: 8, SEQ ID NO: 9 or SEQ ID NO: 13, or a complementary strand thereof. A degenerate sequence designates any nucleotide sequence encoding the same amino acid sequence as a reference nucleotide sequence, but comprising a distinct nucleotide sequence as a result of the genetic code degeneracy. In a preferred embodiment, the nucleic acid molecule is a DNA molecule, such as a double-stranded DNA molecule or a cDNA molecule.

A further object of this invention is a vector comprising DNA encoding any of the above or below described polypeptides. The vector may be any cloning or expression vector, integrative or autonomously replicating, functional in any prokaryotic or eukaryotic cell. In particular, the vector may be a plasmid, cosmid, virus, phage, episome, artificial chromosome, and the like. The vector may comprise regulatory elements, such as a promoter, terminator, enhancer, selection marker, origin of replication, etc. Specific examples of such vectors include prokaryotic plasmids, such as pBR, pUC or pcDNA plasmids; viral vectors, including retroviral, adenoviral or AAV vectors; bacteriophages; baculoviruses; BAC or YAC, etc., as will be discussed below.

The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

B. Host Cells

A further aspect of the present invention is a recombinant host cell, wherein said cell comprises a nucleic acid molecule or a vector as defined above. The host cell may be a prokaryotic or eukaryotic cell. Examples of prokaryotic cells include bacteria, such as E. coli. Examples of eucaryotic cells are yeast cells, plant cells, mammalian cells and insect cells including any primary cell culture or established cell line (e.g., 3T3, Vero, HEK293, TN5, etc.). Suitable host cells for the expression of glycosylated proteins are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CVI line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl, Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). Particularly preferred mammalian cells of the present invention are CHO cells.

C. Production of the Polypeptides and Fusion Proteins of the Present Invention:

Polypeptides and fusion proteins of this invention may be produced by any technique known per se in the art, such as by recombinant technologies, chemical synthesis, cloning, ligations, or combinations thereof. In a particular embodiment, the polypeptides or fusion proteins are produced by recombinant technologies, e.g., by expression of a corresponding nucleic acid in a suitable host cell. Another object of this invention is therefore a method of producing a EPO polypeptide, variant or analog of the present invention (in particular an EPOshort polypeptide or a variant thereof, an EPOshort1 polypeptide or a variant thereof, an EPOshort2 polypeptide or a variant thereof, or an EPOv3 polypeptide or a variant thereof), the method comprising culturing a recombinant host cell of the invention under conditions allowing expression of the nucleic acid molecule, and recovering the polypeptide produced. The polypeptide produced may be glycosylated or not, or may contain other post-translational modifications depending on the host cell type used. Many books and reviews provide teachings on how to clone and produce recombinant proteins using vectors and prokaryotic or eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

The vectors to be used in the method of producing a polypeptide according to the present invention can be episomal or non-/homologously integrating vectors, which can be introduced into the appropriate host cells by any suitable means (transformation, transfection, conjugation, protoplast fusion, electroporation, calcium phosphate-precipitation, direct microinjection, etc.). Factors of importance in selecting a particular plasmid, viral or retroviral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species. The vectors should allow the expression of the polypeptide or fusion proteins of the invention in prokaryotic or eukaryotic host cells, under the control of appropriate transcriptional initiation/termination regulatory sequences, which are chosen to be constitutively active or inducible in said cell. A cell line substantially enriched in such cells can be then isolated to provide a stable cell line.

Host cells are transfected or transformed with expression or cloning vectors described herein for protein production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

For eukaryotic host cells (e.g. yeasts, insect or mammalian cells), different transcriptional and translational regulatory sequences may be employed, depending on the nature of the host. They may be derived form viral sources, such as adenovirus, papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells which have been stably transformed by the introduced DNA can be selected by also introducing one or more markers which allow for selection of host cells which contain the expression vector. The marker may also provide for phototrophy to an auxotrophic host, biocide resistance, e.g. antibiotics, or heavy metals such as copper, or the like. The selectable marker gene can be either directly linked to the DNA sequences to be expressed (e.g., on the same vector), or introduced into the same cell by co-transfection. Additional elements may also be needed for optimal synthesis of proteins of the invention.

Particularly suitable prokaryotic cells include bacteria (such as Bacillus subtilis or E. coli) transformed with a recombinant bacteriophage, plasmid or cosmid DNA expression vector. Such cells typically produce proteins comprising a N-terminal Methionine residue, such proteins representing particular objects of this invention. Preferred cells to be used in the present invention are eukaryotic host cells, e.g. mammalian cells, such as human, monkey (e.g. COS cells), mouse, and Chinese Hamster Ovary (CHO) cells, because they provide post-translational modifications to protein molecules, including correct folding or glycosylation at correct sites. Alternative eukaryotic host cells are yeast cells (e.g., Saccharomyces, Kluyveromyces, etc.) transformed with yeast expression vectors. Also yeast cells can carry out post-translational peptide modifications including glycosylation. A number of recombinant DNA strategies exist which utilize strong promoter sequences and high copy number of plasmids that can be utilized for production of the desired proteins in yeast. Yeast cells recognize leader sequences in cloned mammalian gene products and secrete polypeptides bearing leader sequences (i.e., pre-peptides).

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type. A cell line substantially enriched in such cells can be then isolated to provide a stable cell line.

A particularly preferred method of high-yield production of a recombinant polypeptide of the present invention is through the use of dihydrofolate reductase (DHFR) amplification in DHFR-deficient CHO cells, by the use of successively increasing levels of methotrexate as described in U.S. Pat. No. 4,889,803. The polypeptide obtained may be in a glycosylated form.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines. In the baculovirus system, the materials for baculovirus/insect cell expression systems are commercially available in kit form from, inter alia, Invitrogen.

In addition to recombinant DNA technologies, the polypeptides or fusion proteins of this invention may be prepared by chemical synthesis technologies. Examples of chemical synthesis technologies are solid phase synthesis and liquid phase synthesis. As a solid phase synthesis, for example, the amino acid corresponding to the carboxy-terminus of the polypeptide to be synthesised is bound to a support which is insoluble in organic solvents and, by alternate repetition of reactions (e.g., by sequential condensation of amino acids with their amino groups and side chain functional groups protected with appropriate protective groups), the polypeptide chain is extended. Solid phase synthesis methods are largely classified by the tboc method and the Fmoc method, depending on the type of protective group used. Totally synthetic proteins of size comparable to that of EPO are disclosed in the literature (Brown A et al., 1996).

The polypeptides of the present invention can be produced, formulated, administered, or generically used in other alternative forms that can be preferred according to the desired method of use and/or production. The proteins of the invention can be post-translationally modified, for example by glycosylation. The polypeptides or proteins of the invention can be provided in isolated (or purified) biologically active form, or as precursors, derivatives and/or salts thereof. Said biological activity is at least one of the biological activity described here above.

“Precursors” are compounds which can be converted into the polypeptides of present invention by metabolic and/or enzymatic processing prior to or after administration thereof to cells or an organism. The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the polypeptides of the invention. The term “derivatives” as used herein refers to derivatives that can be prepared from the functional groups present on the lateral chains of the amino acid moieties or on the amino-/or carboxy-terminal groups according to methods known per se in the art. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups. Purification of the polypeptides of the invention can be carried out by a variety of methods known per se in the art, such as, without limitation, any conventional procedure involving extraction, precipitation, chromatography, electrophoresis, or the like. A particular purification procedure is affinity chromatography, using (monoclonal) antibodies or affinity groups which selectively bind the polypeptide and which are typically immobilized on a gel matrix contained within a column. Purified preparations of the proteins of the invention, as used herein, refers to preparations which contain less than 15% of contaminants, more preferably which comprise at least 90, 95 or 97% of the polypeptide.

4. Active Conjugates or Complex:

The polypeptides or fusion proteins of the invention (in particular an EPOshort polypeptide or a variant thereof, an EPOshort1 polypeptide or a variant thereof, an EPOshort2 polypeptide or a variant thereof, or an EPOv3 polypeptide or a variant thereof) can be in the form of active conjugates or complex with a heterologous moiety, which may be selected from cytotoxic agents, labels (e.g. biotin, fluorescent labels), drugs or other therapeutic agents, covalently bound or not, either directly or through the use of coupling agents or linkers. Useful conjugates or complexes can be generated using molecules and methods known per se in the art, for example for allowing the detection of the interaction with the EPO receptor (radioactive or fluorescent labels, biotin), the detection of EPO receptor expressing cells in a sample (radioactive or fluorescent labels, biotin), therapeutic efficacy (cytotoxic agents, drugs or other therapeutic agents). Cytotoxic agents include chemotherapeutic agents, toxin (e.g., an enzymatically active toxin of bacterial, fungal, plant, or animal origin, or fragments thereof), or a radioactive isotope (i.e., a radioconjugate). Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated proteins. Examples include ²¹²Bi, ¹³¹I, ¹³¹In, ⁹⁰Y, and ¹⁸⁶Re.

Useful conjugates or complexes can also be generated for improving the agents in terms of drug delivery efficacy. For this purpose, the polypeptides or fusion proteins of the invention (in particular an EPOshort polypeptide or a variant thereof, an EPOshort1 polypeptide or a variant thereof, an EPOshort2 polypeptide or a variant thereof, or an EPOv3 polypeptide or a variant thereof) can be in the form of active conjugates or complex with molecules such as polyethylene glycol and other natural or synthetic polymers (Harris J M and Chess R B, Nat Rev Drug Discov. (2003), 2(3):214-21; Greenwald R B et al., Adv Drug Deliv Rev. (2003), 55(2):217-50; Pillai O and Panchagnula R, Curr Opin Chem Biol. (2001), 5(4):447-51). In this regard, the present invention contemplates chemically modified polypeptides and proteins as disclosed herein, in which the polypeptide or the protein is linked with a polymer. Typically, the polymer is water soluble so that the conjugate does not precipitate in an aqueous environment, such as a physiological environment. An example of a suitable polymer is one that has been modified to have a single reactive group, such as an active ester for acylation, or an aldehyde for alkylation. In this way, the degree of polymerization can be controlled. An example of a reactive aldehyde is polyethylene glycol propionaldehyde, ormono-(C1-C10) alkoxy, or aryloxy derivatives thereof (see, for example, Harris, et al., U.S. Pat. No. 5,252,714). The polymer may be branched or unbranched. Moreover, a mixture of polymers can be used to produce the conjugates. The conjugates used for therapy can comprise pharmaceutically acceptable water-soluble polymer moieties. Suitable water-soluble polymers include polyethylene glycol (PEG), monomethoxy-PEG, mono-(C1-C10) alkoxy-PEG, aryloxy-PEG, poly-(N-vinyl pyrrolidone) PEG, tresyl monomethoxy PEG, PEG propionaldehyde, bis-succinimidyl carbonate PEG, propylene glycol homopolymers, a polypropyleneoxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A conjugate can also comprise a mixture of such water-soluble polymers.

Examples of conjugates comprise the polypeptide of SEQ ID NO: 13 (EPOv) or amino acids 28 to 55 of SEQ ID NO: 13 (EPOvm), the polypeptide of SEQ ID NO: 15 or amino acids 28 to 55 of SEQ ID NO: 15, the EPOv1 polypeptide of SEQ ID NO: 4 or amino acids 28 to 164 of SEQ ID NO: 4 (EPOv1m), the EPOv2 polypeptide of SEQ ID NO: 6 or amino acids 28 to 104 of SEQ ID NO: 6 (EPOv2m), or the EPOv3 polypeptide of SEQ ID NO: 9 or amino acids 28 to 154 of SEQ ID NO: 9 (EPOv3m), and a polyallcyl oxide moiety attached to the N-terminus of said EPO polypeptide moiety. PEG is one suitable polyalkyl oxide. As an illustration, the EPO polypeptide, variant or analog of the present invention (in particular an EPOshort polypeptide or a variant thereof, an EPOshort1 polypeptide or a variant thereof, an EPOshort2 polypeptide or a variant thereof, or an EPOv3 polypeptide or a variant thereof) can be modified with PEG, a process known as “PEGylation.” PEGylation can be carried out by any of the PEGylation reactions known in the art (see, for example, EP 0 154 316, Delgado et al., Critical Reviews in Therapeutic Drug Carrier Systems 9: 249 (1992), Duncan and Spreafico, Clin. Pharmacokinet. 27: 290 (1994), and Francis et al., Int J Hematol 68: 1 (1998)). For example, PEGylation can be performed by an acylation reaction or by an alkylation reaction with a reactive polyethylene glycol molecule. In an alternative approach, conjugates are formed by condensing activated PEG, in which a terminal hydroxy or amino group of PEG has been replaced by an activated linker (see, for example, Karasiewicz etal., U.S. Pat. No. 5,382,657).

5. Anti-EPO Variant Polypeptide Antibodies:

Some drug candidates for use in the compositions and methods of the present invention are antibodies, antibody fragments or derivative thereof, which selectively bind to any of the above or below described polypeptides.

In a particular embodiment, the antibody, fragment or derivative thereof selectively binds to EPOv and/or an EPOshort polypeptide and/or variant and/or analog of said polypeptide as described here above.

In a particular embodiment, the antibody, fragment or derivative thereof selectively binds to EPOv1 and/or an EPOshort1 polypeptide and/or variant and/or analog of said polypeptide as described here above.

In another particular embodiment, the antibody, fragment or derivative thereof selectively binds to EPOv2 and/or an EPOshort2 polypeptide and/or variant and/or analog of said polypeptide as described here above.

In another embodiment, the antibody, fragment or derivative thereof selectively binds to polypeptides of SEQ ID NO: 8 and/or SEQ ID NO: 9 and/or a variant of said polypeptides as described here above. In a more specific embodiment, the antibody, fragment or derivative thereof selectively binds to EPOv3 and/or a variant of said polypeptide as described here above and distinguishes said polypeptide from EPOwt and/or EPOwtm. Even more specifically, the antibody, fragment or derivative thereof binds to an epitope present in EPOv3 and/or in a variant of said polypeptide as described here above, but absent from EPOwt and/or from EPOwtm. Even more specifically, the antibody, fragment or derivative thereof binds to an epitope located in the C-terminal portion of EPOv3, more particularly this epitope is located into the amino acids 143 to 154 of SEQ ID NO: 9.

Within the context of this invention, the term “selective” binding indicates that the antibodies preferentially bind the target polypeptide or epitope, i.e., with a higher affinity than any binding to any other antigen or epitope. In other words, binding to the target polypeptide can be discriminated from non-specific binding to other antigens. It is preferred that the antibodies according to the present invention exhibit binding affinity (Ka) to the target polypeptide or epitope of 10⁶ M⁻¹ or greater, preferably 10⁷ M⁻¹ or greater, more preferably 10⁸ M⁻¹ or greater and most preferably 10⁹ M⁻¹ or greater. The binding affinity of an antibody can be readily determined by one of ordinary skill in the art, for example, by Scatchard analysis (Scatchard G., Ann NY Acad. Sci. 51: 660-672, 1949).

In a particular embodiment, the antibody, fragment or derivative thereof selectively binds to EPOv and/or an EPOshort polypeptide and/or variant and/or analog of said polypeptide as described here above and distinguishes said polypeptide(s) from EPOwt and/or EPOwtm. Even more specifically, the antibody, fragment or derivative thereof binds to an epitope present in EPOv and/or in EPOshort and/or in a variant and/or in an analog of said polypeptide as described here above, but absent from EPOwt and/or from EPOwtm.

In another particular embodiment, the antibody, fragment or derivative thereof selectively binds to EPOv1 and/or an EPOshort1 polypeptide and/or variant and/or analog of said polypeptide as described here above and distinguishes said polypeptide(s) from EPOwt and/or EPOwtm. Even more specifically, the antibody, fragment or derivative thereof binds to an epitope present in EPOv1 and/or in EPOshort1 and/or in a variant and/or in an analog of said polypeptide as described here above, but absent from EPOwt and/or from EPOwtm.

In another particular embodiment, the antibody, fragment or derivative thereof selectively binds to EPOv2 and/or an EPOshort2 polypeptide and/or variant and/or analog of said polypeptide as described here above and distinguishes said polypeptide(s) from EPOwt and/or EPOwtm. Even more specifically, the antibody, fragment or derivative thereof binds to an epitope present in EPOv2 and/or in EPOshort2 and/or in a variant and/or in an analog of said polypeptide as described here above, but absent from EPOwt and/or from EPOwtm.

Antibodies of this invention may be monoclonal or polyclonal antibodies, or fragments or derivative thereof having substantially the same antigen specificity.

A. Polyclonal Antibodies:

Methods of preparing polyclonal antibodies from various species, including rodents, primates and horses, have been described for instance in Vaitukaitis et al. (J Clin Endocrinol Metab. 33 (1971) p. 988). Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections.

In a particular embodiment, the immunizing agent may include the polypeptide of SEQ ID NO: 13 (EPOv) or amino acids 28 to 55 of SEQ ID NO: 13 (EPOvm), or the polypeptide of SEQ ID NO: 15 or amino acids 28 to 55 of SEQ ID NO: 15, or an EPOshort polypeptide, or a variant or an analog thereof as described hereabove or a fusion protein thereof.

In another particular embodiment, the immunizing agent may include the polypeptide of SEQ ID NO: 4 or amino acids 28 to 164 of SEQ ID NO: 4 (EPOv1m), or an EPOshort1 polypeptide, or a variant or an analog thereof as described hereabove or a fusion protein thereof.

In another particular embodiment, the immunizing agent may include the polypeptide of SEQ ID NO: 6 or amino acids 28 to 104 of SEQ ID NO: 6 (EPOv2m) or an EPOshort2 polypeptide or a variant or an analog thereof as described hereabove or a fusion protein thereof.

In another particular embodiment, the immunizing agent may include the polypeptide of SEQ ID NO: 9 (EPOv3) or amino acids 28 to 154 of SEQ ID NO: 9 (EPOv3m) or a variant as described hereabove or a fusion protein thereof. In a particular embodiment, the immunizing agent may include the polypeptide of SEQ ID NO: 8 or a variant thereof as described hereabove or a fusion protein thereof.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). Repeated injections may be performed. Blood samples are collected and immunoglobulins or serum are separared.

B. Monoclonal Antibodies:

The antibodies may, alternatively, be monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

Methods of producing monoclonal antibodies may be found, for instance, in Harlow et al (Antibodies: A laboratory Manual, CSH Press, 1988) or in Kohler et al (Nature 256 (1975) 495), incorporated therein by reference.

In a hybridoma method, a mouse, hamster, or other appropriate host animal, is typically immunized with an immunizing agent to elicit lymphocytes that produce or are capable of producing antibodies that will specifically bind to the immunizing agent.

In a particular embodiment, the immunizing agent may include the polypeptide of SEQ ID NO: 13 (EPOv) or amino acids 28 to 55 of SEQ ID NO: 13 (EPOvm), or the polypeptide of SEQ ID NO: 15 or amino acids 28 to 55 of SEQ ID NO: 15, or an EPOshort polypeptide, or a variant or an analog thereof as described hereabove or a fusion protein thereof.

In another particular embodiment, the immunizing agent include the polypeptide of SEQ ID NO: 4 or amino acids 28 to 164 of SEQ ID NO: 4 (EPOv1m) or an EPOshort1 polypeptide or a variant or an analog as described hereabove or a fusion protein thereof.

In another particular embodiment, the immunizing agent may include the polypeptide of SEQ ID NO: 6 or amino acids 28 to 104 of SEQ ID NO: 6 (EPOv2m) or an EPOshort2 polypeptide or a variant or an analog as described hereabove or a fusion protein thereof.

In another particular embodiment, the immunizing agent may include the polypeptide of SEQ ID NO: 9 (EPOv3) or amino acids 28 to 154 of SEQ ID NO: 9 (EPOv3m) or a variant as described hereabove or a fusion protein thereof. In a particular embodiment, the immunizing agent may include the polypeptide of SEQ ID NO: 8 or a variant thereof as described hereabove or a fusion protein thereof.

Alternatively, the lymphocytes may be immunized in vitro. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the immunizing peptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purificationproceduressuch as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells.

The “monoclonal antibodies” may also be isolated from phage antibody libraries using the techniques described in Clackson et al., Nature, 352:624-628 [1991] and Marks et al., J. Mol. Biol., 222:581-597 (1991), for example.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Antibodies may also be produced by selection of combinatorial libraries of immunoglobulins, as disclosed for instance in Ward et al (Nature 341 (1989) 544).

C. Human and Humanized Antibodies:

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of non-human species (donor antibody) such as mouse, rat or rabbit having the desired specificity, affinity and capacity. In some instances, Fv framework residues of the human immunoglobulin are replaced by corresponding non-human residues.

Methods for humanizing non-human antibodies are well known in the art. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom and Winter, J. Mol. Biol, 227:381 (1991); Marks et al., J. Mol. Biol, 222:581 (1991)). The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Similarly, human antibodies can be made by the introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10: 779-783 (1992); Lonberg et al., Nature, 368: 856-859 ( 1994); Morrison, Nature, 368: 812-13 (1994); Fishwild et al., Nature Biotechnology, 14:845-51 (1996); Neuberger, Nature Biotechnology, 14: 826 (1996); Lonberg and Huszar, Intern. Rev. Immunol., 13 :65-93 (1995).

D. Immunoconjugates:

The invention also pertains to immunoconjugates comprising an antibody conjugated to heterologous moieties, such as cytotoxic agents, labels, drugs or other therapeutic agents, covalently bound or not, either directly or through the use of coupling agents or linkers. Cytotoxic agent include chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

In another embodiment, the antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

Moreover, antibodies or antibody fragments of the present invention can be PEGylated using methods in the art and described herein. The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

E. Antibody Fragments:

The invention also pertains to “Antibody fragments” which comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng., 8(10): 1057-1062 [1995]); single-chain antibody molecules; monobodies; and multispecific antibodies formed from antibody fragments.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The “light chains” of antibodies (immunoglobulins) from any vertebrate species can be assigned to one of two clearly distinct types, called kappa and lambda, based on the amino acid sequences of their constant domains.

“Single-chain antibody molecules” are fragments of an antibody comprising the VH and VL domains of said antibody, wherein these domains are present in a single polypeptide chain. Preferably, the Fv polypeptide further comprises a polypeptide linker between the VH and VL domains which enables the single-chain antibody molecule to form the desired structure for antigen binding. For a review of single-chain antibody molecules, see, Pluckthun in The Pharmacology of Monoclonal Antibodies, Vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monobodies” as used herein, refers to antigen binding molecules with a heavy chain variable domain and no light chain variable domain. A monobody can bind to an antigen in the absence of light chain and typically has three CDR regions designated CDRH1, CDRH2 and CDRH3. Monobodies include “camelid monobodies” obtained from a source animal of the camelid family, including animals with feet with two toes and leathery soles. Animals in the camelid family include camels, llamas, and alpacas. It has been reported that camels (Camelus dromedaries and Camelus bactrianus) often lack variable light chain domains when material from their serum is analyzed, suggesting that sufficient antibody specificity and affinity can be derived form VH domains (three CDR loops) alone. Monobodies also include modified VH from various animal sources, in particular mammals (for example mouse, rat, rabbit, horse, donkey, bovine or human), which can bind to an antigen in the absence of VL. Preferably, the VH is modified in positions at the VL interface to provide for binding of the VH to antigen in absence of the VL. Davies and Riechmann have for example demonstrated that “camelized monobodies” with high affinity (binding affinity (Ka) to the target polypeptide of 10⁷ M⁻¹ or greater) and high specificity can be generated (Davies & Riechmann, 1995, Biotechnology (NY), 13(5):475-9). Non-specific binding of the VH through its interface for the light chain variable domain (VL) was prevented through three mutations (G44E, L45R and W47G) in this interface. These mutations were introduced to mimic camelid antibody heavy chains naturally devoid of light chain partners.

F. Use of the Antibodies and Antibodies Fragment of the Present Invention:

Antibodies and antibodies fragment of the present invention have various utilities. For example, the antibodies may be used for detecting, dosing, purifying or neutralizing any EPO polypeptide or variant of the present invention described here above. In a particular aspect, the invention thus resides in a method of detecting or dosing a EPO polypeptide or variant of the present invention as defined above in a sample, comprising contacting such a sample with an antibody, fragment or derivative thereof as disclosed above, and determining the formation or dosing the (relative) quantity of an immune complex. The sample may be for instance any biological fluid, such as blood, plasma, serum, etc., optionally diluted and/or treated. The antibody, fragment or derivative thereof may be in suspension or immobilized on a support. The presence or amount of immune complexes may be determined by any technique known per se in the art, e.g., by ELISA, RIA, etc., e.g., using reporter antibodies, labelled antibodies, etc. In a particular embodiment, the antibody, fragment or derivative thereof selectively binds to EPOv and/or an EPOshort polypeptide and/or variant and/or analog of said polypeptide as described here above. In another particular embodiment, the antibody, fragment or derivative thereof selectively binds to EPOv1 and/or an EPOshort1 polypeptide and/or variant and/or analog of said polypeptide as described here above. In another particular embodiment, the antibody, fragment or derivative thereof selectively binds to EPOv2 and/or an EPOshort2 polypeptide and/or variant and/or analog of said polypeptide as described here above. In another embodiment, the antibody, fragment or derivative thereof selectively binds to polypeptides of SEQ ID NO: 8 and/or SEQ ID NO: 9 and/or a variant of said polypeptides as described here above.

Antibodies and antibodies fragment of the present invention are also useful for the affinity purification of EPO polypeptide, variant or analog of the present invention as described here above from recombinant cell culture or natural sources. In this process, the antibodies against the EPO polypeptide, variant or analog of the present invention are immobilized on a suitable support, such a Sephadex resin or filter paper, using methods well known in the art. The immobilized antibody then is contacted with a sample containing the EPO polypeptide, variant or analog to be purified, and thereafter the support is washed with a suitable solvent that will remove substantially all the material in the sample except the EPO polypeptide, variant or analog, which is bound to the immobilized antibody. Finally, the support is washed with another suitable solvent that will release the EPO polypeptide, variant or analog from the antibody. In a particular embodiment, the antibody, fragment or derivative thereof selectively binds to EPOv and/or an EPOshort polypeptide and/or variant and/or analog of said polypeptide as described here above. In another particular embodiment, the antibody, fragment or derivative thereof selectively binds to EPOv1 and/or an EPOshort1 polypeptide and/or variant and/or analog of said polypeptide as described here above. In another particular embodiment, the antibody, fragment or derivative thereof selectively binds to EPOv2 and/or an EPOshort2 polypeptide and/or variant and/or analog of said polypeptide as described here above. In another embodiment, the antibody, fragment or derivative thereof selectively binds to polypeptides of SEQ ID NO: 8 and/or SEQ ID NO: 9 and/or a variant of said polypeptides as described here above.

Antibodies and antibodies fragment of the present invention can be used to block, inhibit, reduce, antagonize or neutralize the activity of the EPO polypeptide, variant or analog of the present invention in particular in the treatment of specific human diseases. Antibodies of the present invention are therefore particularly useful as therapeutic agents. EPO is involved in various physiological actions, including stimulating production of red blood cells (erythrocytes). The EPO receptor (EPOR) is expressed in bone marrow-derived erythroid progenitors and several non-hematopoietic tissues including myocytes, cortical neurons and prostatic, breast and ovarian epithelia. EPO has also been reported to activate specific receptors in the central nervous system and was found to be neurotrophic and neuroprotective in both in vitro and in vivo models.

A further object of this invention is therefore a pharmaceutical composition comprising an antibody or an antibody fragment as defined above, and a pharmaceutically acceptable carrier, excipient, or stabilizer. The present invention also relates to the use of an antibody or an antibody fragment as defined above for the manufacture of a medicament, preferably for treating a human subject. The present invention also pertains to methods of treating, preventing or ameliorating the symptoms of a disorder in a patient, the disorder involving upregulation of EPO expression or activity, the method comprising administering to the patient a pharmaceutical composition comprising an antibody or an antibody fragment as defined above. Within another aspect, the invention provides a method of treating, preventing or ameliorating the symptoms of a cancer in a subject, preferably a human subject, comprising administering a therapeutically effective amount of the antibody as disclosed herein, thereby treating said pathological condition. The invention also pertains to the use of the antibody as disclosed herein in the manufacture of a medicament for the treatment of cancers and tumors. Examples of cancers for treatment include but are not limited to carcinoma including adenocarcinoma, lymphoma, blastoma, sarcoma, melanoma and leukemia. More preferably cancers for treatment herein are kidney cancer such as renal cell carcinoma, in particular metastasizing renal carcinomas and Wilms' tumors, follicular lymphomas, cutaneous T cell lymphomas, Hodgkin's and non-Hodgkin's lymphomas, chronic lymphocytic leukemia and chronic myeloid leukemia, multiple myelomas, tumors that appear following an immune deficiency comprising Kaposi's sarcoma in the case of AIDS, squamous cell cancer, tumors affecting the Central Nervous System, small-cell lung cancer, non-small cell lung cancer, gastrointestinal cancer, pancreatic cancer, cervical cancer, ovarian cancer, liver cancer such as hepatic carcinoma and hepatoma, bladder cancer, breast cancer, colon cancer, colorectal cancer, endometrial carcinoma, salivary gland carcinoma, basal cell carcinoma, melanoma, prostate cancer, vulval cancer, thyroid cancer, testicular cancer, esophageal cancer, and various types of head and neck cancer. Tumors affecting the Central Nervous System include in particular astrocytic tumors (such as anaplastic astrocytoma or glioblastoma), anaplastic oligodendroglioma, anaplastic oligoastrocytoma, medulloblastoma or neuroblastoma. The preferred cancer for treatment herein is adenocarcinoma, more preferably adenocarcinoma of the kidney (such as renal cell carcinoma, in particular metastasizing renal carcinomas and Wilms' tumors), prostate, ovary or breast, or lymphoma (in particular follicular lymphomas, cutaneous T cell lymphomas, Hodgkin's or non-Hodgkin's lymphomas), or leukaemia (in particular chronic lymphocytic leukemia or chronic myeloid leukaemia), or multiple myeloma, or tumors affecting the Central Nervous System (in particular glioblastoma or neuroblastoma).

6. Pharmaceutical Uses of the Polypepltides or Proteins Nucleic Acid Molecules Vector or Cells as Defined Above:

EPO has been involved in various physiological actions. In particular, EPO has been involved in stimulating production of red blood cells (erythrocytes). Recombinant human EPO (rHuEPO) is currently being used to treat patients with anemias associated with chronic renal failure, AIDS patients with anemia due to treatment with zidovudine, nonmyeloid malignancies in patients treated with chemotherapeutic agents, perioperative surgical patients, and autologous blood donation.

EPO has also been reported to activate specific receptors in the central nervous system and was found to be neurotrophic and neuroprotective in both in vitro and in vivo models (Marti H H, Bernaudin M. Function of erythropoietin in the brain. In: Wolfgang J, Ed. Erythropoietin: Molecular Biology and Clinical Use. Johnson City, Tenn.: F. P. Graham Publishing Co., pp 195-215, 2002; Juul S. Acta Paediatr 438 (Suppl):36-42, 2002). EPO and the EPO receptor have both been reported in the brain cortex, cerebellum, hippocampus, pituitary gland and spinal cord. The mechanisms which have been proposed by which EPO produces a neuroprotective effect are: reduction in glutamate toxicity, increased production of neuronal anti-apoptotic factors, reduced nitric oxide mediated injury, anti-inflammatory effects, and anti-oxidant properties.

EPO has also been reported to have a cardioprotective in both in vitro and in vivo models (Calvillo L, et al., (Proc Natl Acad Sci USA. 2003; 100(8):4802-6), Parsa C J et al. (J Clin Invest. 2003. 112(7):999-1007) and Moon C et al. (Proc Natl Acad Sci USA. 2003;100(20):11612-7).

In view of the biological activity of the polyppetides of the present invention (see e.g. section 1.5 here above), the EPO polypeptides, variants and analogs of the present invention are particularly useful in the therapeutic or prophylactic treatment in human subjects.

A further object of this invention is therefore a pharmaceutical composition comprising a polypeptide or protein, nucleic acid, vector or recombinant cell as defined above, and a pharmaceutically acceptable carrier, excipient, or stabilizer. Preferably, the pharmaceutical composition of the present invention comprises an EPO polypeptide or variant or analog of the present invention as defined above.

In a particular embodiment, the pharmaceutical composition of the present invention comprises an EPOshort polypeptide or a variant thereof, an EPOshort1 polypeptide or a variant thereof, an EPOshort2 polypeptide or a variant thereof, or an EPOv3 polypeptide or a variant thereof. In another particular embodiment, the pharmaceutical composition of the present invention comprises an EPOvm (amino acid 28 to 55 of SEQ ID NO: 13) or a variant or an analog as defined here above, or a fusion protein as defined here above comprising such polypeptide. In another particular embodiment, the pharmaceutical composition of the present invention comprises an EPOv1m (amino acid 28 to 164 of SEQ ID NO: 4) or a variant or an analog as defined here above, or a fusion protein as defined here above comprising such polypeptide. In another particular embodiment, the pharmaceutical composition of the present invention comprises an EPOv2m (amino acid 28 to 104 of SEQ ID NO: 6) or a variant or an analog as defined here above, or a fusion protein as defined here above comprising such polypeptide. In another particular embodiment, the pharmaceutical composition of the present invention comprises an EPOv3m (amino acid 28 to 154 of SEQ ID NO: 9) or a variant or an analog as defined here above, or a fusion protein as defined here above comprising such polypeptide.

Another aspect of the present invention relates to the use of a polypeptide or protein, nucleic acid molecule, vector or cell as disclosed above, for the manufacture of a medicament, preferably for treating a human subject.

The present invention also pertains to methods of treating, preventing or ameliorating the symptoms of a disorder in a patient, preferably a human subject, the disorder involving disregulation of EPO expression or activity, the method comprising administering to the patient a pharmaceutical composition as defined above. Preferably, the method comprises administering to the patient a therapeutically effective amount of an EPO polypeptide, variant or analog of the present invention as disclosed hereabove. In a particular embodiment, the polypeptide is an EPOshort polypeptide or a variant thereof, the polypeptide is an EPOshort1 polypeptide or a variant thereof, an EPOshort2 polypeptide or a variant thereof, or an EPOv3 polypeptide or a variant thereof. In another particular embodiment, the polypeptide comprises or consists of EPOvm (amino acid 28 to 55 of SEQ ID NO: 13) or a variant or an analog as defined here above, or a fusion protein as defined here above comprising such polypeptide. In another particular embodiment, the polypeptide comprises or consists of EPOv1m (amino acid 28 to 164 of SEQ ID NO: 4) or a variant or an analog as defined here above, or a fusion protein as defined here above comprising such polypeptide. In another particular embodiment, the polypeptide comprises or consists of EPOv2m (amino acid 28 to 104 of SEQ ID NO: 6) or a variant or an analog as defined here above, or a fusion protein as defined here above comprising such polypeptide. In another particular embodiment, the polypeptide comprises or consists of EPOv3m (amino acid 28 to 154 of SEQ ID NO: 9) or a variant or an analog as defined here above, or a fusion protein as defined here above comprising such polypeptide.

In another aspect, the invention provides a method of treating, preventing or ameliorating the symptoms of a disorder in a patient, preferably a human subject, wherein the disorder is selected from the group consisting of: blood disorders characterized by low or defective red blood cell production, anemia, Chronic Renal Failure patients hypertension, surgery patients, Pediatric patients on dialysis, diseases or conditions associated with insufficient hematocrit levels, AIDS, disorders connected with chemotherapy treatments, cancers and tumors, infectious diseases, venereal diseases, immunologically related diseases and/or autoimmune diseases and disorders, cardiovascular diseases such as stroke, hypotension, cardiac arrest, ischemia in particular ischemia-reperfusion injury, myocardial infarction such as acute myocardial infarctions, chronic heart failure, angina, cardiac hypertrophy, cardiopulmonary diseases, heart-lung bypass, respiratory diseases, kidney, urinary and reproductive diseases, endocrine and metabolic abnormalities, gastrointestinal diseases, diseases of the central nervous system (CNS) or peripheral nervous system which have primarily neurological or psychiatric symptoms, age-related loss of cognitive function, cerebral palsy, neurodegenerative disease, Alzheimer's disease, Parkinson's disease, Leigh's disease, dementia, memory loss, amyotrophic lateral sclerosis, alcoholism, mood disorder, anxiety disorder, attention deficit disorder, hyperactivity, autism, schizophrenia, depression, brain or spinal cord trauma or ischemia, Creutzfeld-Jakob disease, ophthalmic diseases, seizure disorder, multiple sclerosis, inflammation, radiation damage, macular degeneration, diabetic neuropathy, diabetic retinopathy, glaucoma, retinal ischemia, and retinal trauma, the method comprising administering to the patient a therapeutically effective amount of a polypeptide or a pharmaceutical composition of the present invention. In a further aspect, the invention contemplates the use of a polypeptide or a pharmaceutical composition of the present invention in the manufacture of a medicament for the treatment of a disorder in a patient preferably a human subject the disorder being selected from the group consisting of: blood disorders characterized by low or defective red blood cell production, anemia, Chronic Renal Failure patients hypertension, surgery patients, Pediatric patients on dialysis, diseases or conditions associated with insufficient hematocrit levels, AIDS, disorders connected with chemotherapy treatments, cancers and tumors, infectious diseases, venereal diseases, immunologically related diseases and/or autoimmune diseases and disorders, cardiovascular diseases such as stroke, hypotension, cardiac arrest, ischemia in particular ischemia-reperfusion injury, myocardial infarction such as acute myocardial infarctions, chronic heart failure, angina, cardiac hypertrophy, cardiopulmonary diseases, heart-lung bypass, respiratory diseases, kidney, urinary and reproductive diseases, endocrine and metabolic abnormalities, gastrointestinal diseases, diseases of the central nervous system (CNS) or peripheral nervous system which have primarily neurological or psychiatric symptoms, age-related loss of cognitive function, cerebral palsy, neurodegenerative disease, Alzheimer's disease, Parkinson's disease, Leigh's disease, dementia, memory loss, amyotrophic lateral sclerosis, alcoholism, mood disorder, anxiety disorder, attention deficit disorder, hyperactivity, autism, schizophrenia, depression, brain or spinal cord trauma or ischemia, Creutzfeld-Jakob disease, ophthalmic diseases, seizure disorder, multiple sclerosis, inflammation, radiation damage, macular degeneration, diabetic neuropathy, diabetic retinopathy, glaucoma, retinal ischemia, and retinal trauma.

In an embodiment, disorders for treatment are anemia, Chronic Renal Failure patients hypertension, Pediatric patients on dialysis, diseases or conditions associated with insufficient hematocrit levels, disorders connected with chemotherapy treatments, cancers, cardiovascular diseases, diseases of the central nervous system (CNS) or peripheral nervous system which have primarily neurological or psychiatric symptoms.

In a preferred embodiment, disorders for treatment are diseases of the central nervous system (CNS). In a preferred embodiment, said disorders for treatment are acute ischemic stroke, or brain or spinal cord trauma or ischemia. In yet another preferred embodiment, said disorders for treatment are neurodegenerative diseases such as Alzheimer's disease, or Parkinson's disease. In yet another preferred embodiment, said disorder for treatment is selected in the group consisting of: schizophrenia, epilepsy, coronary bypass damage and subarachnoid hemorrhage. In yet another preferred embodiment the disease for treatment herein is multiple sclerosis or Alzheimer's disease, or Parkinson's disease. In yet another preferred embodiment, said disorder for treatment is Multiple Sclerosis.

In another preferred embodiment, disorders for treatment are diseases of the peripheral nervous system. In a preferred embodiment, said disorders for treatment are neuropathy, diabetic neuropathy, vertebral disk compression or carpal tunnel syndrome.

In another preferred embodiment, disorder for treatment is neuropathic pain. In a preferred embodiment, disorder for treatment is neuropathic pain associated with alcoholism, and/or amputation, and/or sciatica, and/or cancer chemotherapy, and/or diabetes and/or facial nerve problems (trigeminal neuralgia), and/or HIV infection or AIDS, and/or Multiple sclerosis and/or shingles (herpes zoster virus infection) and/or spine surgery.

Preferably the cardiovascular disease treated is ischemia in particular ischemia-reperfusion injury or myocardial infarction such as acute myocardial infarctions. In another particular embodiment, the cardiovascular disease treated is chronic heart failure.

In another preferred embodiment, disorders for treatment are diseases of the retina. In a preferred embodiment, said disorders for treatment are diabetic retinopathy, retinal trauma, macular degeneration, retinal ischemia or diabetic macular edema.

In another preferred embodiment, disorders for treatment are diseases of the kidney. In a preferred embodiment, said disorders for treatment are diabetic nephropathy, kidney toxic injury, transplant or renal failure.

Examples of cancers for treatment herein include but are not limited to carcinoma including adenocarcinoma, lymphoma, blastoma, sarcoma, melanoma and leukemia. More preferably cancers for treatment herein are kidney cancer such as renal cell carcinoma, in particular metastasizing renal carcinomas and Wilms' tumors, follicular lymphomas, cutaneous T cell lymphomas, Hodgkin's and non-Hodgkin's lymphomas, chronic lymphocytic leukemia and chronic myeloid leukemia, multiple myelomas, tumors that appear following an immune deficiency comprising Kaposi's sarcoma in the case of AIDS, squamous cell cancer, tumors affecting the Central Nervous System, small-cell lung cancer, non-small cell lung cancer, gastrointestinal cancer, pancreatic cancer, cervical cancer, ovarian cancer, liver cancer such as hepatic carcinoma and hepatoma, bladder cancer, breast cancer, colon cancer, colorectal cancer, endometrial carcinoma, salivary gland carcinoma, basal cell carcinoma, melanoma, prostate cancer, vulval cancer, thyroid cancer, testicular cancer, esophageal cancer, and various types of head and neck cancer. Tumors affecting the Central Nervous System include in particular astrocytic tumors (such as anaplastic astrocytoma or glioblastoma), anaplastic oligodendroglioma, anaplastic oligoastrocytoma, medulloblastoma or neuroblastoma. The preferred cancer for treatment herein is adenocarcinoma, more preferably adenocarcinoma of the kidney (such as renal cell carcinoma, in particular metastasizing renal carcinomas and Wilms' tumors), prostate, ovary or breast, or lymphoma (in particular follicular lymphomas, cutaneous T cell lymphomas, Hodgkin's or non-Hodgkin's lymphomas), or leukaemia (in particular chronic lymphocytic leukemia or chronic myeloid leukaemia), or multiple myeloma, or tumors affecting the Central Nervous System (in particular glioblastoma or neuroblastoma).

Examples of infectious diseases for treatment herein include viral infections comprising chronic hepatitis B and C and HIV/AIDS, infectious pneumonias, and venereal diseases, such as genital warts.

Examples of immunologically and auto-immunologically related diseases for treatment herein include the rejection of tissue or organ grafts, allergies, asthma, psoriasis, rheumatoid arthritis, multiple sclerosis, Crohn's disease and ulcerative colitis.

In an embodiment, the disease for treatment herein is anemia, more preferably anemia associated with Chronic Renal Failure (CRF), anemia in Zidovudine-treated HIV-infected patients, anemia in cancer patients on Chemotherapy or radiotherapy, anemia associated with the progression of non-myeloid cancers, anemia associated with viral infection (such as HIV) or anemia of chronic disease.

The polypeptides or pharmaceutical compositions of the present invention may also be used for the preparation of a therapeutic compound intended to increase the production of autologous blood, notably in patients participating in a differed autologous blood collection program to avoid the use of blood from another person (this is for example the case when loss of blood is envisaged during surgery).

The pharmaceutical compositions of the present invention may contain, in combination with the polypeptides or proteins of the invention as active ingredient, suitable pharmaceutically acceptable diluents, carriers, biologically compatible vehicles and additives which are suitable for administration to an animal (for example, physiological saline solution) and optionally comprising auxiliaries (like excipients, stabilizers, or adjuvants) which facilitate the processing of the active compounds into preparations which can be used pharmaceutically. The pharmaceutical compositions may be formulated in any acceptable way to meet the needs of the mode of administration. For example, the use of biomaterials and other polymers for drug delivery, as well the different techniques and models to validate a specific mode of administration, are disclosed in literature (Luo B and Prestwich G D, 2001; Cleland J L et al., Curr Opin Biotechnol. (2001), 12(2):212-9). “Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

The pharmaceutical composition may be in a liquid or lyophilized form and comprises a diluent (Tris, citrate, acetate or phosphate buffers) having various pH values and ionic strengths, solubilizer such as Tween or Polysorbate, carriers such as human serum albumin or gelatin, preservatives such as thimerosal, parabens, benzylalconium chloride or benzyl alcohol, antioxidants such as ascrobic acid or sodium metabisulfite, and other components such as lysine or glycine. Selection of a particular composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980). In a preferred embodiment, the EPO polypeptides and variants of the invention are formulated in liquid form in an isotonic sodium chloride/sodium citrate buffered solution containing human albumin, and optionally containing benzyl alcohol as a preservative.

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, rectal, oral, or buccal routes. Preferably the pharmaceutical compositions of the invention are administered by injection, either subcutaneous or intravenous. The route of administration eventually chosen will depend upon a number of factors and may be ascertained by one skilled in the art.

The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose.

For instance, in the case of the treatment of anemia (such as anemia associated with CRF, anemia in Zidovudine-treated HIV-infected patients, anemia in cancer patients on Chemotherapy or radiotherapy, anemia associated with the progression of non-myeloid cancers, anemia associated with viral infection (such as HIV) or anemia of chronic disease), the dosing frequency for an EPO polypeptide or variant or analog of the present invention will vary depending upon the condition being treated and the target hematocrit, but in general will be less than three times per week. The dosing frequency will be about two times per week, about one time per week. The dosing frequency may also be less than about one time per week, for example about one time every two weeks (about one time per 14 days), one time per month or one time every two months. It is understood that the dosing frequencies actually used may vary somewhat from the frequencies disclosed herein due to variations in responses by different individuals to the EPO polypeptide or variant or analog; the term “about” is intended to reflect such variations. In the case of the treatment of anemia with an EPO polypeptide or variant or analog of the present invention the therapeutically effective amount refers to an amount of a EPO polypeptide or variant or analog (or a fusion protein as described here above) which gives an increase in hematocrit to a target hematocrit, or to a target hematocrit range that provides benefit to a patient or, alternatively, maintains a patient at a target hematocrit, or within a target hematocrit range. The amount will vary from one individual to another and will depend upon a number of factors, including the overall physical condition of the patient, severity and the underlying cause of anemia and ultimate target hematocrit for the individual patient. A target hematocrit is typically in a range of 30%-60%, or in a range of 30%-45%, and more preferably 40%-45%. It is understood that such targets will vary from one individual to another such that physician discretion may be appropriate in determining an actual target hematocrit for any given patient. Nonetheless, determining a target hematocrit is well within the level of one skilled in the art.

The pharmaceutical composition of the present invention may be administered alone or in conjunction with other therapeutics directed to the condition, or directed to other symptoms of the condition. Usually a dose range for once per week administration of an EPO polypeptide or variant or analog according to the present invention is from about 0,05 to about 10 μg erythropoietin peptide per kg per dose. A dose range for three times per week administration would usually be 0.02 to 2.5 μg per kg per dose. Subsequent administrations can be performed at a dosage, which is the same, less than, or greater than the initial or previous dose administered to the individual.

In a preferred embodiment, the EPO polypeptide or variant or analog of the present invention is a therapeutic with greater potency than rHuEPO. An advantage to such a composition is that it could be administered less frequently and/or at a lower dose. Current treatments for patients suffering from anemia call for administration of EPO three times per week and for surgery patients administration once per day. A less frequent dosing schedule would be more convenient to both physicians and patients, especially those patients who do not make regularly scheduled visits to doctor's offices or clinics, or those who self-inject their EPO. Another advantage of a more potent molecule is that less drug is being introduced into patients for a comparable increase in hematocrit. Therefore, in a preferred embodiment, the EPO polypeptide or variant or analog of the present invention are more potent molecules for the treatment of anemia compared to rHuEPO which will permit a less frequent dosing schedule. In another preferred embodiment, the EPO polypeptide or variant or analog of the present invention will increase and maintain hematocrit at levels which are at least comparable to that of rHuEPO when administered at a lower dose. Yet in another preferred embodiment, the EPO polypeptide or variant or analog of the present invention are at least as well tolerated as rHuEpo and potentially better tolerated in some patients.

In yet a very preferred embodiment, the EPO polypeptide or variant or analog of the present invention have a tissue protective activity in mammal in particular in human, without substantially increasing hematocrit level in said mammal. Such polypeptides are particularly advantageous as they can be administered with having less effect compared to wild type EPO or even no effect on the hematocrit level of the patient to be treated. An advantage to such polypeptides is that they could be administered more frequently and/or at a higher dose compared to wild type EPO without increasing hematocrit to a too high level with the associated side effects and risks. Such polypeptide or variant or analog of the present invention are potentially better tolerated in patients.

7. Detection of Nucleic Acids Coding for an EPO Polypeptide or Variant of the Present Invention:

A further aspect of the present invention relates to compositions and methods for detecting or dosing a nucleic acid, preferably RNA or cDNA, coding for an EPO polypeptide or variant or analog of the present invention in a sample. Such compositions include, for instance, any specific nucleic acid probes or primers which specifically recognise a nucleic acid encoding the EPO polypeptide or variant or analog hereabove described.

A particular embodiment is directed to fragments of a nucleic acid sequence coding an EPO polypeptide or variant or analog according to the present invention that may find use as hybridization probes. Such nucleic acid fragments are from 20 through 80 nucleotides in length, preferably from 20 through 60 nucleotides in length, more preferably 20 through 50 nucleotides in length, and most preferably from 20 through 38 nucleotides in length.

In a particular embodiment, the hybridization probes is derived from at least partially a sequence coding for a polypeptide of SEQ ID NO: 4 or a variant or analog of said polypeptide as defined hereabove or a complementary strand thereof. In a further particular embodiment, the hybridization probe is a nucleic acid from 20 through 38 nucleotides in length coding for a polypeptide of SEQ ID NO: 4 or a complementary strand thereof, and even more preferably a nucleic acid from 20 through 38 nucleotides of SEQ ID NO: 5 or a complementary strand thereof.

In another particular embodiment, the hybridization probes is derived from at least partially a sequence coding for a polypeptide of SEQ ID NO: 6 or a variant or analog of said polypeptide as defined hereabove or a complementary strand thereof. In a further particular embodiment, the hybridization probe is a nucleic acid from 20 through 38 nucleotides in length coding for a polypeptide of SEQ ID NO: 6 or a complementary strand thereof, and even more preferably a nucleic acid from 20 through 38 nucleotides of SEQ ID NO: 7 or a complementary strand thereof.

In another particular embodiment, the hybridization probes is derived from at least partially a sequence coding for a polypeptide of SEQ ID NO: 8, or SEQ ID NO: 9, or a variant of said polypeptides as defined hereabove or a complementary strand thereof. In a further preferred embodiment, the hybridization probe is a nucleic acid from 20 through 36 or 38 nucleotides in length coding for a polypeptide of SEQ ID NO: 8, or SEQ ID NO: 9, or a complementary strand thereof, and even more preferably a nucleic acid from 20 through 36 or 38 nucleotides of SEQ ID NO: 10, or SEQ ID NO: 11, or a complementary strand thereof.

In another particular embodiment, the hybridization probes is derived from at least partially a sequence coding for a polypeptide of SEQ ID NO: 13 or a variant or analog of said polypeptide as defined hereabove or a complementary strand thereof. In a further particular embodiment, the hybridization probe is a nucleic acid from 20 through 38 nucleotides in length coding for a polypeptide of SEQ ID NO: 13 or a complementary strand thereof, and even more preferably a nucleic acid from 20 through 38 nucleotides of SEQ ID NO: 12 or a complementary strand thereof.

In this regard, the term “nucleic acid molecule” encompasses all different types of nucleic acids, including without limitation deoxyribonucleic acids (e.g., DNA, cDNA, gDNA, synthetic DNA, etc.), ribonucleic acids (e.g., RNA, mRNA, etc.) and peptide nucleic acids (PNA). In a preferred embodiment, the nucleic acid molecule is a DNA molecule, such as a double-stranded DNA molecule or a cDNA molecule. Hybridization probes may be labelled by a variety of labels, including radionucleotides such as ¹²P, ³³P or ³⁵S, or enzymatic labels such as alkaline phosphatase coupled to the probe via avidin/biotin coupling systems. Labeled probes having a sequence complementary to that of the cDNA of the present invention can be used to screen libraries of human cDNA, genomic DNA or mRNA to determine which members of such libraries the probe hybridizes to. Hybridization techniques are well known in the art.

Other useful fragments of a nucleic acid sequence coding an EPO polypeptide or variant or analog according to the present invention, include antisense or sense oligonucleotides comprising a single-stranded nucleic acid sequence (either RNA or DNA) capable of binding to target mRNA (sense) or DNA (antisense) sequences. In a particular embodiment, said fragments are fragments of a nucleic acid coding for a polypeptide of SEQ ID NO: 4 or a variant or analog of said polypeptide as defined hereabove or a complementary strand thereof, or of a nucleic acid of SEQ ID NO: 5 or a complementary strand thereof. In another particular embodiment, said fragments are fragments of a nucleic acid coding for a polypeptide of SEQ ID NO: 6 or a variant or analog of said polypeptide as defined hereabove or a complementary strand thereof, or of a nucleic acid of SEQ ID NO: 7 or a complementary strand thereof. In another particular embodiment, said fragments are fragments of a nucleic acid coding for a polypeptide of SEQ ID NO: 13 or a variant or analog of said polypeptide as defined hereabove or a complementary strand thereof, or of a nucleic acid of SEQ ID NO: 12 or a complementary strand thereof. In another particular embodiment, said fragments are fragments of a nucleic acid sequence coding for a polypeptide of SEQ ID NO: 8, or SEQ ID NO: 9, or a variant of said polypeptide as defined hereabove or a complementary strand thereof, or of a nucleic acid of SEQ ID NO: 10, or SEQ ID NO: 11, or a complementary strand thereof.

Antisense or sense oligonucleotides, according to the present invention, comprise a fragment of the coding region of EPO DNA. Such a fragment generally comprises at least 14 nucleotides, preferably from 14 to 36 or 38 nucleotides, even more preferably from 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988). Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of EPO proteins (in particular EPO polypeptide or variant or analog according to the present invention) and more particularly to block expression of a specific transcriptional variant, in particular the expression of EPOv1, EPOv2 or EPOv3. In a particular embodiment, the antisense oligonucleotides of the present invention are chosen such as to bind to the mRNA encoding EPOv1 in the region of the junction between the exon 2 and 4 (such as overlapping the junction). In another particular embodiment, the antisense oligonucleotides of the present invention are chosen such as to bind to the mRNA encoding EPOv2 in the region of the junction between the exon 2 and 5 (such as overlapping the junction).

A further use of the fragments of the nucleic acid sequence as defined here above is their use as primers as to amplify at least a distinctive fragment of a nucleic acid molecule encoding an EPO polypeptide or variant or analog as defined above. A “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase. Typical primers of the present invention are single-stranded nucleic acid molecules of 6 to 50 nucleotides in length, more preferably of 8 to 40 nucleotides in length, even more preferably of 12 to 30 nucleotides in length. The sequence of the primer can be derived directly from the sequence of the target nucleic acid molecule. Perfect complementarity between the primer sequence and the target gene is preferred, to ensure high specificity. However, certain mismatch may be tolerated. In a particular embodiment the primer is of 12 to 30 nucleotides, more preferably is of 15 to 20 nucleotides in length and is a fragment of SEQ ID NO: 5 or its complementary strand. These primers are particularly suitable as RT-PCR primers to specifically amplify the transcriptional variant EPOv1 when chosen such as to bind to the cDNA encoding EPOv1 in the region of the junction between the exon 2 and 4 (such as overlapping the junction) and associated to another primer chosen upstream or downstream by methods known in the art. In another particular embodiment the primer is of 12 to 30 nucleotides, more preferably is of 15 to 20 nucleotides in length and is a fragment of SEQ ID NO: 7 or its complementary strand. These primers are particularly suitable as RT-PCR primers to specifically amplify the transcriptional variant EPOv2 when chosen such as to bind to the cDNA encoding EPOv1 in the region of the junction between the exon 2 and 5 (such as overlapping the junction) and associated to another primer chosen upstream or downstream by methods known in the art. In yet another particular embodiment the primer is of 12 to 30 nucleotides, more preferably is of 15 to 20 nucleotides in length and is a fragment of SEQ ID NO: 11 or its complementary strand. These primers are particularly suitable as RT-PCR primers to specifically amplify a transcriptional variant encoded at least in part by the 3′ end of exon 4A, when associated to another primer chosen upstream or downstream by methods known in the art. In another particular embodiment the primer is of 12 to 30 nucleotides, more preferably is of 15 to 20 nucleotides in length and is a fragment of SEQ ID NO: 12 or its complementary strand or of SEQ ID NO: 14 or its complementary strand.

The present invention also relates to the RNA interference (RNAi) technology. RNA interference (RNAi) refers to a mechanism of post-transcriptional gene silencing (PTGS) in which double-stranded RNA (dsRNA) corresponding to a gene or mRNA of interest is introduced into an organism resulting in the degradation of the corresponding mRNA. In the RNAi reaction, both the sense and anti-sense strands of a dsRNA molecule are processed into small RNA fragments or segments ranging in length from 19 to 25 nucleotides (nt), preferably 21 to 23 nt, and having 2-nucleotide 3′ tails. These dsRNAs are known as “guide RNAs” or “short interfering RNAs” (siRNAs). siRNAs can also include short hairpin RNAs (shRNAs) in which both strands of an siRNA duplex are included within a single RNA molecule. Alternatively, synthetic dsRNAs, which are 19 to 25 nt in length, preferably 21 to 23 nt, and have 2-nucleotide 3′ tails, can be synthesized, purified and used in the reaction. The siRNA duplexes then bind to a nuclease complex composed of proteins that target and destroy endogenous mRNAs having homology to the siRNA within the complex. In this manner, specific mRNAs can be targeted and degraded, thereby resulting in a loss of protein expression from the targeted mRNA. The specific requirements and modifications of dsRNA are described in PCT Publication No. WO01/75164 (incorporated herein by reference). While dsRNA molecules can vary in length, it is preferable to use siRNA molecules which are 19- to 25-nt in length, most preferably 21- to 23-nucleotides in length, and which have characteristic 2- to 3- nucleotide 3′ overhanging ends typically either (2′-deoxy) thymidine or uracil. The siRNAs typically comprise a 3′ hydroxyl group. Single stranded siRNA as well as blunt ended forms of dsRNA can also be used. In order to further enhance the stability of the RNA, the 3′ overhangs can be stabilized against degradation. In one such embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium. siRNA can be prepared using any of the methods known in the art including those set forth in PCT Publication No. WO01/75164 or using standard procedures for in vitro transcription of RNA and dsRNA annealing procedures as described in Elbashir et al. (Genes & Dev., 15:188-200, 2001). In the present invention, the dsRNA, or siRNA, is substantially complementary to at least a part of the mRNA sequence of an EPO polypeptide or variant or analog mRNA as described herein and can reduce or inhibit the expression or biological activity of the EPO variant described herein. Desirably, the siRNA is 100% complementary to 18 to 25 consecutive nucleotides of the EPO variants described herein (in particular EPOv1, EPOv2 or EPOv3). Preferably, the decrease in the EPO polypeptide or variant or analog described herein biological activity is at least 5% relative to cells treated with a control dsRNA, shRNA, or siRNA, more preferably at least 10%, 20%, or 25%, and most preferably at least 50%. Methods for assaying levels of protein expression are also well known in the art and include western blotting, immunoprecipitation, and ELISA. Methods for assaying the EPO polypeptides and variants or analogs biological activity include assays described herein.

All patent and literature references cited in the present specification are hereby incorporated by reference in their entirety.

Further aspects and advantages of the present invention will be disclosed in the following examples, which should be considered as illustrative only, and do not limit the scope of this application.

### EXAMPLES

### Example 1

### Cloning of a Transcriptional Variant of EPO Encoded by Exons 1, 2, 4 and 5 of the Human Gene EPO

An EPO transcriptional variant (EPOv1) encoded by exons 1, 2, 4 and 5 was predicted in the Human gene of EPO (see FIG. 4). Our prediction leads to an EPOv1 protein encoded in 164 amino acids (SEQ ID NO: 4), corresponding to 492 bp spanning 4 exons. The prediction contained an initiating methionine, a signal sequence and a stop codon (FIG. 4).

In order to generate EPOv1 protein the exons can be amplified from genomic DNA by PCR. The amplified exons are then re-assembled by cloning techniques well known in the art. The PCR product corresponding to the EPOv1 coding sequence (FIG. 4) is then subcloned into a mammalian expression vector.

Another possibility for the production of the EPOv1 protein is the cloning from a pool of RNA as will be described here below.

1.1: Cloning of EPOv1 Encoded by Exons 1, 2, 4 and 5 from a Pool of RNA.

EPOv1 has been cloned from a pool of RNA using reverse transcription and cloning techniques. The pool of RNA used is a mix of RNA from different tissues. The mix used was the following: polyA RNA of human pancreas (Clontech; catalogue reference number: 636119), polyA RNA of human skeletal muscle (Clontech; catalogue reference number: 636120), polyA RNA of human small intestine (Clontech; catalogue reference number: 636125), polyA RNA of human testis (Clontech; catalogue reference number: 636115), polyA RNA of human liver (Clontech; catalogue reference number: 636101), polyA RNA of human brain (Whole) (Clontech; catalogue reference number: 636102) and total RNA of human normal adipose (Invitrogen tissue collection, (lot A5040004), InVitrogen, Carlsbad, Calif., U.S.A.).

1.1.1 cDNA Synthesis (Production of Pool)

A) First-Strand cDNA Synthesis:

For the cDNA synthesis the SMART™ RACE cDNA Amplification Kit from Clontech (Mountain View, Calif., catalog reference number: 634914) has been used, following the manufacturer's recommendations. The protocol used was the following:
1—Mix preparation:
0.5 μl of the pool of RNA (see above)
1 μl 3′ SMART CDSPrimer II A (10 μM)
1 μl SMART II A Oligonucleotide (10 μm)
2.5 μl Deionized H₂O
   2—Mix contents and spin the tube briefly in a microcentrifuge
   3—Incubate at 72° c for 2 min
   4—Cool the tube on ice for 2 min
   5—Add the following to each reaction tube:
2 μl 5×First-Strand Buffer
1 μl DTT (20 mM)
1 μl 50×dNTP (10 mM)
1 μl PowerScript Reverse Transcriptase
   6—Incubate the tubes at 42° C. for 1 hour
   7—Add 190 μl of TE 1× (pH 7.5)
   8—Incubate at 72° C. 7 min
   9—Stock at −20° C.

B) Advantage-GC PCR Protocol

A PCR using the Advantage GC 2 PCR Kit & Polymerase Mix from Clontech (Mountain View, Calif., catalog reference number: 639119) has been performed. The protocol used is the following:
1—Mix preparation:
29 μl H₂O
10 μl 5×GCX 2 PCR buffer
5 μl GC Melt (5M)
2 μl Nested universal primer A 10 mM
1 μl 50×dNTP (10 mM each)
1 μl Advantage-GC Pol. Mix
   2—Add 2 μl of the product obtain in step A)
   3—PCR reaction:

| | | | | |
|---|---|---|---|---|
| 94° C. | 1 | min | | 1 cycle |
| 94° C. | 15 | sec | | |
| 65° C. | 5 | sec | {close oversize parenthesis} | 20 cycles |
| 68° C. | 12 | min | | |
| 68° C. | 12 | min | | 1 cycle |

4—Tube for using: 0.4 ng/μl.

1.1.2 Cloning of EPOv1 cDNA:

The first stage of the Gateway cloning process (Gateway PCR cloning system commercially available from Invitrogen) involves a two step PCR reaction which generates the ORF of EPOv1 flanked at the 5′ end by an attB1 recombination site and Kozak sequence, and flanked at the 3′ end by a sequence encoding an in frame 6 Histidine (6His) tag, a stop codon and the attB2 recombination site (Gateway compatible cDNA) using the cDNA produced here above as template.

A) First PCR

A first PCR using RecF1 (TGAGGGACCCCGGCCAGGCGCGGAG (SEQ ID NO: 16)) and RecR1 (ATGCCCAGGTGGACACACCTGGTCA (SEQ ID NO: 17)) as primers, the product obtain in step B) here above as matrix and the following conditions has been performed:
1—Mix preparation

| | |
|---|---|
| 5 μl of product obtain in step | |
| B) here above | |
| 45 μl H₂O | |
| 5 μl Buffer “TaqPlus ® | Stratagene (La Jolla, CA, catalog |
| Precision” 10X | reference number: 600211) |
| 0.4 μl dNTP 25 mM | Invitrogen (Carlsbad, CA, catalog |
| | reference number 10297-018) |
| 1 μl primers at 10 μM each | RecF1 and RecR1 (see above) |
| 0.25 μl TaqPlus ® Precision | Stratagene (La Jolla, CA, catalog |
| | reference number: 600211) |
| 2.5 μl DMSO 100% | |

2—PCR reaction:

| | | | | |
|---|---|---|---|---|
| 94° c. | 1 | min | | 1 cycle |
| 94° C. | 40 | sec | | |
| 45° C. | 40 | sec | {close oversize parenthesis} | 3 cycles |
| 72° C. | 1 | min | | |
| 94° C. | 40 | sec | | |
| 55° C. | 40 | sec | {close oversize parenthesis} | 9 cycles |
| 72° C. | 1 | min | | |
| 72° C. | 5 | min | | |

| | |
|---|---|
| 4° C. | to finish |

B) Second PCR

A second PCR using cloF1

(GGGGACAAGTTTGTACAAAAAAGCAGGCTTCGCCACCATGGGGGTGCACG AATGTCC (SEQ ID NO: 18)) and cloR1

(GGGGACCACTTTGTACAAGAAAGCTGGGTTTCAATGGTGATGGTGATGGTG TCTGTCCCCTGTCCTGCAGG (SEQ ID NO: 19)) as primers, the product obtain in step A) here above as matrix and the following conditions has been performed:
1—Mix preparation:

| | |
|---|---|
| 10 μl of the product obtain in step | |
| A) here above | |
| 31.5 μl H₂O | |
| 4 μl Buffer “TaqPlus ® | Stratagene (La Jolla, CA, catalog |
| Precision” 10X | reference number: 600211) |
| 0.32 μl dNTP 25 mM | Invitrogen (Carlsbad, CA, catalog |
| | reference number 10297-018) |
| 4 μl primers at 10 μM each | cloF1 and cloR1 (see above) |
| 0.2 μl TaqPlus ® Precision | Stratagene (La Jolla, CA, catalog |
| | reference number: 600211) |

2—PCR reaction:

| | | | | |
|---|---|---|---|---|
| 94° c. | 1 | min | | 1 cycle |
| 94° C. | 40 | sec | | |
| 45° C. | 40 | sec | {close oversize parenthesis} | 3 cycles |
| 72° C. | 1 | min | | |
| 94° C. | 1 | min | | |
| 94° C. | 40 | sec | | |
| 50° C. | 40 | sec | {close oversize parenthesis} | 3 cycles |
| 72° C. | 1 | min | | |
| 94° C. | 40 | sec | | |
| 55° C. | 40 | sec | {close oversize parenthesis} | 7 cycles |
| 72° C. | 1 | min | | |
| 72° C. | 5 | min | | |

| | |
|---|---|
| 4° C. | to finish |

C) BP Reaction

The second stage of the Gateway cloning process (Gateway entry cloning by BP recombination, Invitrogen) involves subcloning of the Gateway modified PCR product (ie. The PCR product obtain at step B) here above) into the Gateway entry vector pDONR™ 201.
1—Mix preparation:

| | |
|---|---|
| 1- Mix preparation: | |
| 1 μl of the product obtain in step | |
| B) here above | |
| 4 μl TE10.1 | |
| 1 μl vector pDONR201(300 ng/μl) | Invitrogen (Carlsbad, CA, catalog |
| | reference number 11798-014) |
| 2 μl buffer BP reaction 5X | Invitrogen (Carlsbad, CA, catalog |
| | reference number 11789-013) |
| 2 μl BP clonase enzyme | Invitrogen (Carlsbad, CA, catalog |
| | reference number 11789-013) |
| 2- incubate 1 hour at 25° c. | |
| 3- add 1 μl of proteinase K | Merck KGaA (Darmstadt, Germany |
| | catalog reference number 1.24568) |
| 4- incubate 10 min at 37° C. | |
| 5- stock at 4° C. | |

D) Transformation

The product of the reaction obtained at step C) here above has been used to transform E. coli DH10B cells by electroporation.

1-Mix Composition:
1 μl of BP-reaction product obtained at step C) here above

| | |
|---|---|
| 30 μl of ElectroMax DH10B cells | Invitrogen (Carlsbad, CA, catalog |
| | reference number 18290-015) |

The mixture was transferred to a chilled 0.1 cm electroporation cuvette
and the cells electroporated according to the manufacturer's protocol.
500 μl of SOC medium was added immediately after electroporation.
2-incubate 1 hour at 37° C., under agitation
3-spread 30 μl on LB agar plate (+50 μg/ml of Kanamycine)
4-incubate 37° C. over night.

Splice Variant Identification:

96 clones were picked and used to innoculate 150 μl cultures of LB+Kanamycin, and incubated at 37° C. for 20 h.

2 microliters of each culture was used for PCR with pDONR specific primers (forward primer, 5′-TCG CGT TAA CGC TAG CAT GGA TCT C-3′ (SEQ ID NO: 32); reverse primer, 5′-GTA ACA TCA GAG ATT TTG AGA CAC-3′ (SEQ ID NO: 33))
PCR conditions:
PCR mix:
2 μl culture
10×PCR buffer 2 μl
20 mM dNTP 0.2 μl
10 μM pDONR forward primer 1 μl
10 μM pDONR reverse primer 1 μl
Taq DNA polymerase (5 U/μl) 0.1 μl
Add ddH₂O to 20 μl

PCR program

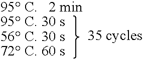

5 μl of PCR product were run on a 50 cm 2% agarose gel in TAE at 5V/cm for 3 hours. PCR products displaying a fragment size different from the canonical expected size were selected (5 ml culture) for sequencing and confirmation of alternative splice variant structure.

Plasmid mini-prep DNA was prepared from 5 ml cultures from some of the resultant colonies and subjected to DNA sequencing. Plasmid DNA (1.5 μl or approx. 100 ng) from one of the clones, which contained the correct sequence (pDONR201_EPOv1-HIS), was then used in recombination reactions containing 1.5 μl of pEAK12d vector (0.1 μg/μl), 2 μl LR buffer and 1.5 μl of LR clonase (Invitrogen) in a final volume of 10 μl. The mixtures were incubated at room temperature for 1 hour. The reactions were stopped by addition of Proteinase K (2 μg) and incubated at 37° C. for a further 10 minutes. An aliquot of each reaction (1 μl) was used to transform E. coli DH10B cells by electroporation. Aliquots of the transformation mixture were plated on L-broth (LB) plates containing ampicillin (100 μg/ml) and incubated overnight at 37° C.

Plasmid mini-prep DNA was prepared from 5 ml cultures from some of the resultant colonies. Plasmid DNA (200-500 ng) in the pEAK12d vector was subjected to DNA sequencing. Plasmid maxi-prep DNA was prepared from a 500 ml culture of the sequence verified clones (pEAK12d_EPOv1-HIS) using Qiagen Plasmid MEGA Kit (QIAGEN) according to the manufacturer's instructions. Plasmid DNA was resuspended at a concentration of 1 μg/μl in sterile water (or 10 mM Tris-HCl pH 8.5) and stored at −20° C.

A sequence of the plasmid pEAK12d_EPOv1-HIS is given at SEQ ID NO: 20.

### Example 2

### Expression of EPOv1 (His-tagged)

Human cells, e.g. human Embryonic Kidney 293 cells expressing the Epstein-Barr virus Nuclear Antigen (HEK293-EBNA, Invitrogen) were transfected with the expression vector allowing the expression of EPOv1 in such cells (pEAK12d_EPOv1-HIS). The cells expressing EPOv1 were grown and the recombinant protein was extracted from the culture medium.

2.1 Functional Genomics Expression in Mammalian Cells of the Cloned EPOv1 (His-tagged)

Human Embryonic Kidney 293 cells expressing the Epstein-Barr virus Nuclear Antigen (HEK293-EBNA, Invitrogen) were routinely maintained in suspension in Ex-cell VPRO serum-free medium (seed stock, maintenance medium, JRH). Prior to transfection, 500 μg EPOv1-coding plasmid DNA (pEAK12d_EPOv1-HIS) and 10 μg reporter-gene plasmid was added to 50 ml FEME 1% FBS. Then 1 ml PEI (1 mg/ml Polysciences, USA) was added. Following agitation, the mix was incubated for 10 minutes at room temperature. The cell inoculum was resuspended with the transfection-mix solution and added to 200 ml FEME (DMEM/Ham's F-12 1:1, complemented to 19 mM HEPES, 5 g/L Glucose, 7.5 mM L-Glutamine, 4 ml/L ITS-X) (all Invitrogen-Life Technologies) medium supplemented with 1% FBS (Invitrogen) to reach a cell density of 1×10⁶ cells/ml in a suitable vessel. The culture was further incubated at 37° C. in an incubator with 5% CO₂ atmosphere and at least 70% relative humidity for 90 min. Finally, the volume was topped up with 250ml chemically defined, serum-free FreeStyle 293 (Invitrogen) medium complemented with 4 ml/L ITS-X. The transfected culture was further incubated in the same conditions as for transfection for 6 days. At day of harvest, confirmation of positive transfection was done by qualitative fluorescence examination (Axiovert 10 Zeiss). Supernatant (500 ml) was centrifuged (1800×g, 4° C., 6-10 min), sterile-filtered through a 0.22 um filter unit (Millipore, 500 ml filter unit) and purified by IMAC (Immobilised Metal Affinity Chromatography) chromatography. One aliquot (500 μl) of the supernatant was kept for QC of the 6His-tagged protein.

2.2 Purification of the Cloned EPOv1 (His-tagged)

The 500 ml culture medium sample containing the EPOv1 recombinant protein with a C-terminal 6His tag was diluted with one volume cold buffer A (50 mM NaH2PO4; 600 mM NaCl; 8.7% (w/v) glycerol, pH 7.5) to a final volume of 1000 ml. The sample was filtered through a 0.22 mm sterile filter (Millipore, 500 ml filter unit) and kept at 4° C. in a 1 liter sterile square media bottle (Nalgene). The purification was performed at 4° C. on a VISION workstation (Applied Biosystems) connected to an automatic sample loader (Labomatic). The purification procedure was composed of two sequential steps, metal affinity chromatography on a Poros 20 MC (Applied Biosystems) column charged with Ni ions (10×50 mm, 3.93 ml), followed by buffer exchange on a Sephadex G-25 medium (Amersham Pharmacia) gel filtration column (1,0×15 cm). For the first chromatography step the metal affinity column was regenerated with 30 column volumes of EDTA solution (100 mM EDTA; 1 M NaCl; pH 8.0), recharged with Ni ions through washing with 15 column volumes of a 100 mM NiSO4 solution, washed with 10 column volumes of buffer A, followed by 7 column volumes of buffer B (50 mM NaH2PO4; 600 mM NaCl; 8.7% (w/v) glycerol, 400 mM; imidazole, pH 7.5), and finally equilibrated with 15 column volumes of buffer A containing 15 mM imidazole. The sample was transferred, by the Labomatic sample loader, into a 200 ml sample loop and subsequently charged onto the Ni metal affinity column at a flow rate of 20 ml/min. The charging procedure was repeated 5 times in order to transfer the entire sample (1000 ml) onto the Ni column. Subsequently the column was washed with 12 column volumes of buffer A, followed by 28 column volumes of buffer A containing 20 mM imidazole. During the 20 mM imidazole wash loosely attached contaminating proteins were eluted of the column. The recombinant EPOv1 His-tagged protein was finally eluted with 10 column volumes of buffer B at a flow rate of 2 ml/min, and the eluted protein was collected in a 2.7 ml fraction. For the second chromatography step, the Sephadex G-25 gel-filtration column was regenerated with 2 ml of buffer D (1.137 M NaCl; 2.7 mM KCl; 1.5 mM KH2PO4; 8 mM Na2HPO4; pH 7.2), and subsequently equilibrated with 4 column volumes of buffer C (137 mM NaCl; 2.7 mM KCl; 1.5 mM KH2PO4; 8 mM Na2HPO4; 20% (w/v) glycerol; pH 7.4). The peak fraction eluted from the Ni-column was automatically, through the integrated sample loader on the VISION, loaded onto the Sephadex G-25 column and the protein was eluted with buffer C at a flow rate of 2 ml/min. The desalted sample was recovered in a 2.7 ml fraction. The fraction was filtered through a 0.22 mm sterile centrifugation filter (Millipore), aliquoted, frozen and stored at −80° C. An aliquot of the sample was analyzed on SDS-PAGE (4-12% NuPAGE gel; Novex) by Coomassie blue staining and Western blot with anti-His antibodies. A further aliquot was taken for determination of the level LPS endotoxin.

Coomassie Blue staining. The NuPAGE gel was stained in a 0.1% coomassie blue R250 staining solution (30% methanol, 10% acetic acid) at room temperature for 1 h and subsequently destained in 20% methanol, 7.5% acetic acid until the background was clear and the protein bands clearly visible.

Western blot. Following the electrophoresis the proteins were electrotransferred from the gel to a nitrocellulose membrane at 290 mA for 1 hour at 4° C. The membrane was blocked with 5% milk powder in buffer E (137 mM NaCl; 2.7 mM KCl; 1.5 mM KH2PO4; 8 mM Na2HPO4; 0.1% Tween 20, pH 7.4) for 1 h at room temperature, and subsequently incubated with a mixture of 2 rabbit polyclonal anti-His antibodies (G-18 and H-15, 0.2ug/ml each; Santa Cruz) in 2.5% milk powder in buffer E overnight at 4° C. After further 1 hour incubation at room temperature, the membrane was washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane was developed with the ECL kit (Amersham) for 1 min. The membrane was subsequently exposed to a Hyperfilm (Amersham), the film developed and the Western blot image visually analyzed.

Protein assay. The protein concentration was determined using the BCA protein assay kit (Pierce) with bovine serum albumin as standard. The yield was 520 mg purified EPOv1-6HIS.

Assay for LPS. LPS content was estimated using The Endosafe—Portable Test System (Charles River PTS100) according to the makers instructions. Samples were tested in quadruplicate and LPS was expressed as U/mg protein. LPS level of EPOv1 was determined to be 5.32 U/mg which is acceptable for injection into animals.

### Example 3

### Cloning of a Variant of EPO Encoded by Exons 1, 2 and 5 of the Human Gene EPO (EPOv2)

A sequence containing exons 1, 2 and 5 of the Human gene of EPO and encoding EPOv2 (see FIG. 5) was identified and cloned using the procedure described in Example 1. A sequence of the expression plasmid pEAK12d_EPOv2-HIS obtained is given at SEQ ID NO: 21.

### Example 4

### Expression of EPOv2 (His-tagged)

EPOv2 protein is 104 amino acids long (SEQ ID NO: 6), corresponding to 312 bp spanning 3 exons. The sequence contains an initiating methionine, a signal sequence and a stop codon (FIG. 5).

Human cells, e.g. human Embryonic Kidney 293 cells expressing the Epstein-Barr virus Nuclear Antigen (HEK293-EBNA, Invitrogen) were transfected with the expression vector allowing the expression of EPOv2 in such cells (pEAK12d_EPOv2-HIS). The protein was produced according to the protocol as described at example 2.

### Example 5

### Cloning of a Transcriptional Variant of EPO Encoded by Exons 1, 2, 3 and a Longer Exon 4 (Named Herein Exon 4A) of the Human Gene EPO

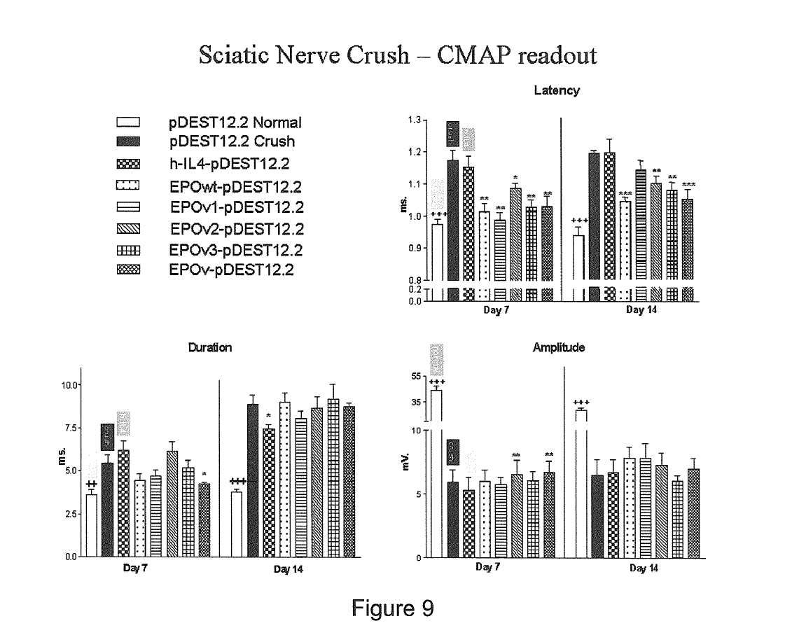

Testing the effects of EPOv1 and EPOv2 in sciatic nerve crush revealed that both have activities similar to that found for the wild type protein in this test (see Example 11 and FIG. 9). The variant proteins share two domains in common with the full length molecule, namely those encoded by exon 2 and by exon 5. In an attempt to define more accurately the region of the protein which confers protection in the sciatic nerve crush model, we have investigated the activity of a variant protein EPOv3. This variant lacks exon 5 and was cloned as described below.

An EPO variant (EPOv3) encoded by exons 1, 2, 3 and 4A was predicted in the Human gene of EPO. Said exon 4A is longer at the 3′ end as compared to exon 4 which encode the wild-type EPO (see FIGS. 3 and 6). Our prediction leads to an EPOv3 protein encoded in 154 amino acids (SEQ ID NO: 9), corresponding to 462 bp spanning 4 exons (the new exon identified has been named exon 4A). The prediction contained an initiating methionine, a signal sequence and a stop codon (FIG. 6).

5.1: Cloning of EPOv3 Encoded by Exons 1, 2, 3 and a Slightly Extended Exon 4 from a Pool of RNA.

EPOv3 has been cloned from a pool of RNA using reverse transcription and cloning techniques. The pool of RNA used is a mix of RNA from different tissues. The mix used was the following: polyA RNA of human pancreas (Clontech; catalogue reference number: 636119), polyA RNA of human skeletal muscle (Clontech; catalogue reference number: 636120), polyA RNA of human small intestine (Clontech; catalogue reference number: 636125), polyA RNA of human testis (Clontech; catalogue reference number: 636115), polyA RNA of human liver (Clontech; catalogue reference number: 636101), polyA RNA of human brain (Whole) (Clontech; catalogue reference number: 636102) and total RNA of human normal adipose (Invitrogen tissue collection, (lot A5040004), InVitrogen, Carlsbad, Calif., U.S.A.).

5.1.1 cDNA Synthesis (Production of Pool)

A) First-Strand cDNA Synthesis:

For the cDNA synthesis the SMART™ RACE cDNA Amplification Kit from Clontech (Mountain View, Calif., catalogue reference number: 634914) has been used following the manufacturer's recommendations. The protocol used was the following:
1—Mix preparation:
0.5 μl of the pool of RNA (see above)
1 μl 3′ SMART CDSPrimer II A (10 μM)
1 μl SMART II A Oligonucleotide (10 μm)
2.5 μl Deionized H₂O
   2—Mix contents and spin the tube briefly in a microcentrifuge
   3—Incubate at 72° C. for 2 min
   4—Cool the tube on ice for 2 min
   5—Add the following to each reaction tube:
2 μl 5×First-Strand Buffer
1 μl DTT (20 mM)
1 μl 50×dNTP (10 mM)
1 μl PowerScript Reverse Transcriptase
   6—Incubate the tubes at 42° C. for 1 hour
   7—Add 190 μl of TE 1× (pH 7.5)
   8—Incubate at 72° C. 7 min
   9—Stock at −20° C.

B) Advantage-GC PCR Protocol

A PCR using the Advantage GC 2 PCR Kit & Polymerase Mix from Clontech (Mountain View, Calif., catalog reference number: 639119) has been performed. The protocol used is the following:
1—Mix preparation:
29 μl H₂O
10 μl 5×GCX 2 PCR buffer
5 μl GC Melt (5M)
2 μl Nested universal primer A 10 mM
1 μl 50×dNTP (10 mM each)
1 μl Advantage-GC Pol. Mix
   2—Add 2 μl of the product obtain in step A)
   3—PCR reaction:

| | | | | |
|---|---|---|---|---|
| 94° C. | 1 | min | | 1 cycle |
| 94° C. | 15 | sec | | |
| 65° C. | 5 | sec | {close oversize parenthesis} | 20 cycles |
| 68° C. | 12 | min | | |
| 68° C. | 12 | min | | 1 cycle |

4—Tube for using: 0.4 ng/μl.

5.1.2 Cloning of EPOv3 cDNA:

The first stage of the Gateway cloning process (Gateway PCR cloning system commercially available from Invitrogen) involves a two step PCR reaction which generates the ORF of EPOv3 flanked at the 5′ end by an attB1 recombination site and Kozak sequence, and flanked at the 3′ end by a sequence encoding an in frame 6 Histidine (6His) tag, a stop codon and the attB2 recombination site (Gateway compatible cDNA) using the cDNA produced here above as template.

A) First PCR

A first PCR using RecF1 (TGAGGGACCCCGGCCAGGCGCGGAG (SEQ ID NO: 16)) and RecR1 (ATGCCCAGGTGGACACACCTGGTCA (SEQ ID NO: 17)) as primers, the product obtain in step B) here above as matrix and the following conditions has been performed:
1—Mix preparation

| | |
|---|---|
| 5 μl of product obtain in step | |
| B) here above | |
| 45 μl H₂O | |
| 5 μl Buffer “TaqPlus ® | Stratagene (La Jolla, CA, catalog |
| Precision” 10X | reference number: 600211) |
| 0.4 μl dNTP 25 mM | Invitrogen (Carlsbad, CA, catalog |
| | reference number 10297-018) |
| 1 μl primers at 10 μM each | RecF3 and RecR3 (see above) |
| 0.25 μl TaqPlus ® Precision | Stratagene (La Jolla, CA, catalog |
| | reference number: 600211) |
| 2.5 μl DMSO 100% | |

2—PCR reaction:

| | | | | |
|---|---|---|---|---|
| 94° c. | 1 | min | | 1 cycle |
| 94° C. | 40 | sec | | |
| 45° C. | 40 | sec | {close oversize parenthesis} | 3 cycles |
| 72° C. | 1 | min | | |
| 94° C. | 40 | sec | | |
| 55° C. | 40 | sec | {close oversize parenthesis} | 9 cycles |
| 72° C. | 1 | min | | |
| 72° C. | 5 | min | | |

| | |
|---|---|
| 4° C. | to finish |

B) Second PCR

A second PCR using cloF1

(GGGGACAAGTTTGTACAAAAAAGCAGGCTTCGCCACCATGGGGGTGCACG AATGTCC (SEQ ID NO: 18)) and cloR3

(GGGGACCACTTTGTACAAGAAAGCTGGGTTTCAATGGTGATGGTGATGGTG CAGAAAGGGCAAGCAGAAGT (SEQ ID NO: 22)) as primers, the product obtain in step A) here above as matrix and the following conditions has been performed:
1—Mix preparation:

| | |
|---|---|
| 10 μl of the product obtain in step | |
| A) here above | |
| 31.5 μl H₂O | |
| 4 μl Buffer “TaqPlus ® | Stratagene (La Jolla, CA, catalog |
| Precision” 10X | reference number: 600211) |
| 0.32 μl dNTP 25 mM | Invitrogen (Carlsbad, CA, catalog |
| | reference number 10297-018) |
| 4 μl primers at 10 μM each | cloF3 and cloR3 (see above) |
| 0.2 μl TaqPlus ® Precision | Stratagene (La Jolla, CA, catalog |
| | reference number: 600211) |

2—PCR reaction:

| | | | | |
|---|---|---|---|---|
| 94° c. | 1 | min | | 1 cycle |
| 94° C. | 40 | sec | | |
| 45° C. | 40 | sec | {close oversize parenthesis} | 3 cycles |
| 72° C. | 1 | min | | |
| 94° C. | 1 | min | | |
| 94° C. | 40 | sec | | |
| 50° C. | 40 | sec | {close oversize parenthesis} | 3 cycles |
| 72° C. | 1 | min | | |
| 94° C. | 40 | sec | | |
| 55° C. | 40 | sec | {close oversize parenthesis} | 7 cycles |
| 72° C. | 1 | min | | |
| 72° C. | 5 | min | | |

| | |
|---|---|
| 4° C. | to finish |

C) BP reaction

The second stage of the Gateway cloning process (Gateway entry cloning by BP recombination, Invitrogen) involves subcloning of the Gateway modified PCR product (ie. The PCR product obtain at step B) here above) into the Gateway entry vector pDONR™ 201.
1—Mix preparation:

| | |
|---|---|
| 1 μl of the product obtain in step | |
| B) here above | |
| 4 μl TE10.1 | |
| 1 μl vector pDONR201(300 ng/μl) | Invitrogen (Carlsbad, CA, catalog |
| | reference number 11798-014) |
| 2 μl buffer BP reaction 5X | Invitrogen (Carlsbad, CA, catalog |
| | reference number 11789-013) |
| 2 μl BP clonase enzyme | Invitrogen (Carlsbad, CA, catalog |
| | reference number 11789-013) |
| 2- incubate 1 hour at 25° c. | |
| 3- add 1 μl of proteinase K | Merck KGaA (Darmstadt, Germany |
| | catalog reference number 1.24568) |
| 4- incubate 10 min at 37° C. | |
| 5- stock at 4° C. | |

D) Transformation

The product of the reaction obtained at step C) here above has been used to transform E. coli DH10B cells by electroporation.
1—Mix composition:
1 μl of BP-reaction product obtained at step C) here above
30 μl of ElectroMax DH10B cells

Invitrogen (Carlsbad, Calif., catalog reference number 18290-015)

The mixture was transferred to a chilled 0.1 cm electroporation cuvette and the cells electroporated according to the manufacturer's protocol.
500 μl of SOC medium was added immediately after electroporation.
   2—incubate 1 hour at 37° C., under agitation
   3—spread 30 μl on LB agar plate (+50 μg/ml of Kanamycine)
   4—incubate 37° C. over night.

Plasmid mini-prep DNA was prepared from 5 ml cultures from some of the resultant colonies and subjected to DNA sequencing. Plasmid DNA (1.5 μl or approx. 100 ng) from one of the clones, which contained the correct sequence (pDONR201_EPOv3-HIS), was then used in recombination reactions containing 1.5 μl of pEAK12d vector (0.1 μg/μl), 2 μl LR buffer and 1.5 μl of LR clonase (Invitrogen) in a final volume of 10 μl. The mixtures were incubated at room temperature for 1 hour. The reactions were stopped by addition of Proteinase K (2 μg) and incubated at 37° C. for a further 10 minutes. An aliquot of each reaction (1 μl) was used to transform E. coli DH10B cells by electroporation. Aliquots of the transformation mixture were plated on L-broth (LB) plates containing ampicillin (100 mg/ml) and incubated overnight at 37° C.

Plasmid mini-prep DNA was prepared from 5 ml cultures from some of the resultant colonies. Plasmid DNA (200-500 ng) in the pEAK12d vector was subjected to DNA sequencing. Plasmid maxi-prep DNA was prepared from a 500 ml culture of the sequence verified clones (pEAK12d_EPOv3-HIS) using Qiagen Plasmid MEGA Kit (QIAGEN) according to the manufacturer's instructions. Plasmid DNA was resuspended at a concentration of 1 μg/μl in sterile water (or 10 mM Tris-HCl pH 8.5) and stored at −20° C.

A sequence of the plasmid pEAK12d_EPOv3-HIS is given at SEQ ID NO: 23.

### Example 6

### Expression of EPOv3 (His-tagged)

Human cells, e.g. human Embryonic Kidney 293 cells expressing the Epstein-Barr virus Nuclear Antigen (HEK293-EBNA, Invitrogen) are transfected with the expression vector allowing the expression of EPOv3 in such cells (pEAK12d_EPOv3-HIS). The cells expressing EPOv3 are grown and the recombinant protein is extracted from the culture medium.

6.1 Functional Genomics Expression in Mammalian Cells of the Cloned EPOv3 (His-tagged)

Human Embryonic Kidney 293 cells expressing the Epstein-Barr virus Nuclear Antigen (HEK293-EBNA, Invitrogen) are routinely maintained in suspension in Ex-cell VPRO serum-free medium (seed stock, maintenance medium, JRH). Prior to transfection, 500 μg EPO-coding plasmid DNA (pEAK12d_EPOv3-HIS) and 10 μg reporter-gene plasmid is added to 50 ml FEME 1% FBS. Then 1 ml PEI (1 mg/ml Polysciences, USA) is added. Following agitation, the mix is incubated for 10 minutes at room temperature. The cell inoculum is resuspended with the transfection-mix solution and added to 200 ml FEME (DMEM/Ham's F-12 1:1, complemented to 19 mM HEPES, 5 g/L Glucose, 7.5 mM L-Glutamine, 4 ml/L ITS-X) (all Invitrogen-Life Technologies) medium supplemented with 1% FBS (Invitrogen) to reach a cell density of 1×10⁶ cells/ml in a suitable vessel. The culture is further incubated at 37° C. in an incubator with 5% CO₂ atmosphere and at least 70% relative humidity for 90 min. Finally, the volume is topped up with 250 ml chemically defined, serum-free FreeStyle 293 (Invitrogen) medium complemented with 4 ml/L ITS-X. The transfected culture is further incubated in the same conditions as for transfection for 6 days. At day of harvest, confirmation of positive transfection is done by qualitative fluorescence examination (Axiovert 10 Zeiss). Supernatant (500 ml) is centrifuged (1800×g, 4° C., 6-10 min), sterile-filtered through a 0.22 um filter unit (Millipore, 500 ml filter unit) and purified by IMAC (Immobilised Metal Affinity Chromatography) chromatography. One aliquot (500 μl) of the supernatant is kept for QC of the 6His-tagged protein.

6.2 Purification of the Cloned EPOv3 (His-tagged)

The 500 ml culture medium sample containing the EPOv3 recombinant protein with a C-terminal 6His tag is diluted with one volume cold buffer A (50 mM NaH2PO4; 600 mM NaCl; 8.7% (w/v) glycerol, pH 7.5) to a final volume of 1000 ml. The sample is filtered through a 0.22 mm sterile filter (Millipore, 500 ml filter unit) and kept at 4° C. in a 1 liter sterile square media bottle (Nalgene). The purification is performed at 4° C. on a VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (10×50 mm, 3.93 ml), followed by buffer exchange on a Sephadex G-25 medium (Amersham Pharmacia) gel filtration column (1,0×15 cm). For the first chromatography step the metal affinity column is regenerated with 30 column volumes of EDTA solution (100 mM EDTA; 1 M NaCl; pH 8.0), recharged with Ni ions through washing with 15 column volumes of a 100 mM NiSO4 solution, washed with 10 column volumes of buffer A, followed by 7 column volumes of buffer B (50 mM NaH2PO4; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 20 ml/min. The charging procedure is repeated 5 times in order to transfer the entire sample (1000 ml) onto the Ni column. Subsequently the column is washed with 12 column volumes of buffer A, followed by 28 column volumes of buffer A containing 20 mM imidazole. During the 20 mM imidazole wash loosely attached contaminating proteins are eluted of the column. The recombinant EPOv3 His-tagged protein is finally eluted with 10 column volumes of buffer B at a flow rate of 2 ml/min, and the eluted protein is collected in a 2.7 ml fraction. For the second chromatography step, the Sephadex G-25 gel-filtration column is regenerated with 2 ml of buffer D (1.137 M NaCl; 2.7 mM KCl; 1.5 mM KH2PO4; 8 mM Na2HPO4; pH 7.2), and subsequently equilibrated with 4 column volumes of buffer C (137 mM NaCl; 2.7 mM KCl; 1.5 mM KH2PO4; 8 mM Na2HPO4; 20% (w/v) glycerol; pH 7.4). The peak fraction eluted from the Ni-column is automatically, through the integrated sample loader on the VISION, loaded onto the Sephadex G-25 column and the protein is eluted with buffer C at a flow rate of 2 ml/min. The desalted sample is recovered in a 2.7 ml fraction. The fraction is filtered through a 0.22 mm sterile centrifugation filter (Millipore), aliquoted, frozen and stored at −80° C. An aliquot of the sample is analyzed on SDS-PAGE (4-12% NuPAGE gel; Novex) by Coomassie blue staining and Western blot with anti-His antibodies.

Coomassie Blue staining. The NuPAGE gel is stained in a 0.1% coomassie blue R250 staining solution (30% methanol, 10% acetic acid) at room temperature for 1 h and subsequently destained in 20% methanol, 7.5% acetic acid until the background is clear and the protein bands clearly visible.

Western blot. Following the electrophoresis the proteins are electrotransferred from the gel to a nitrocellulose membrane at 290 mA for 1 hour at 4° C. The membrane is blocked with 5% milk powder in buffer E (137 mM NaCl; 2.7 mM KCl; 1.5 mM KH2PO4; 8 mM Na2HPO4; 0.1% Tween 20, pH 7.4) for 1 h at room temperature, and subsequently incubated with a mixture of 2 rabbit polyclonal anti-His antibodies (G-18 and H-15, 0.2 ug/ml each; Santa Cruz) in 2.5% milk powder in buffer E overnight at 4° C. After further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham) for 1 min. The membrane is subsequently exposed to a Hyperfilm (Amersham), the film developed and the Western blot image visually analyzed.

Protein assay. The protein concentration is determined using the BCA protein assay kit (Pierce) with bovine serum albumin as standard.

### Example 7

### Tissue Distribution of the Human EPO Variant

The expression pattern of the predicted EPOv1, EPOv2 and EPOv3 mRNA are determined using RT-PCR analysis. cDNA templates of various tissues are amplified using variant specific primers, to determine tissue expression of the variants.

### Example 8

### Cloning of a Truncated Variant of EPO Encoded by Exons 1, 2, and the First Two Amino Acids of Exon 3 of the Human Gene EPO (Named Herein EPOv and EPOvm in its Mature Form)

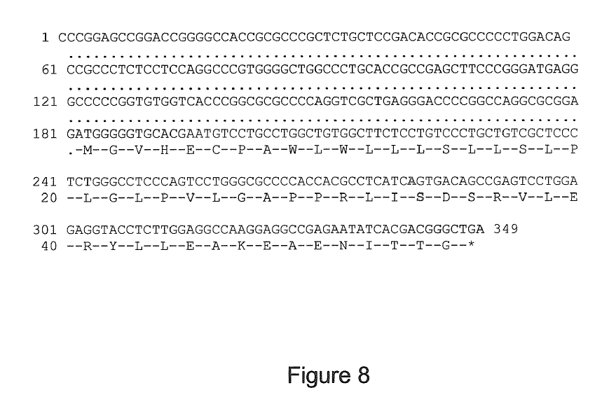

Oligonucleotide directed deletion mutagenesis was performed to generate the sequence shown at SEQ ID NO: 12. Four oligonucleotide primers AS671 to AS674 (see table I) were used to perform PCR reactions as follows: In PCR reactions 1 and 2, the template DNA was full length wild type erythropoietin cDNA cloned into pDEST12.2 expression vector (Invitrogen cat. No. 11808011). In reaction 1, primers AS671 and AS674 were used to amplify the N-terminal part of the sequence shown in FIG. 8; in reaction 2, primers AS672 and AS673 were used to amplify the C-terminal part of the sequence shown in FIG. 8. In both cases, reaction mixtures were set up containing 1×PCR buffer, 0.2 mM each dNTP, 0.5 mM each PCR primer, 50 ng template DNA, and the reaction was initiated by addition of 5U PfuTurbo (Stratagene). Cycling conditions were: 95° C. 2 min (1 cycle); 95° C. 15 sec, 50° C. 30 sec, 72° C. 70 sec (25 cycles); 75° C. 7 min (1 cycle). An aliquot of each PCR reaction was analysed by electrophoresis in 0.8% agarose gels to estimate the PCR efficiency and yield. In a third PCR reaction, equimolar amounts the overlapping products of reactions 1 and 2 were mixed together and amplified in the presence of PCR primers AS671 and AS672 to generate the full length 0.9 kb fragment. PCR reaction conditions were as described above for reactions 1 and 2. An aliquot of the purified PCR reaction was digested with NdeI (New England Biolabs) for 2 h at 37° C. using the enzyme buffer supplied by the manufacturer. In parallel, an appropriate amount of the pDEST12.2 expression vector was digested with NdeI. The digested vector and insert were each separated on a 0.8% agarose gel, the corresponding fragments were excised and purified using the Wizard Cleanup System (Promega) according to the protocol provided by the manufacturer. The purified vector DNA and PCR product were mixed in a molar ratio of 1:3 and precipitated overnight at −20° C. by addition of 2.5 volumes ethanol. The precipitated DNA was recovered by centrifugation, washed in 70% ethanol, dried under vacuum and ligated in a final volume of 10 μl using the Rapid Ligation Kit (Roche Diagnostics) according to the protocol supplied by the manufacturers.

The ligation mixture was then used to transform E. coli strain JM101 as follows: 50 μl aliquots of competent JM101 cells were thawed on ice and 1μl or 5 μl of the ligation mixture reaction was added. The cells were incubated for 40 min on ice and then heat shocked by incubation at 42° C. for 2 min. 1 ml of warm (room temperature) L-Broth (LB) was added and samples were incubated for a further 1 h at 37° C. The transformation mixture was then plated on LB plates containing ampicillin (100 μg/ml) and incubated overnight at 37° C. Single colonies were picked for plasmid isolation.

Plasmid DNA preparation, restriction digestion and sequence analysis.

Miniprep plasmid DNA was prepared from 5 ml cultures using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices).

Aliquots of miniprep plasmid DNAs (100-200 ng) were digested with NdeI for 2 h at 37° C. and analysed by electrophoresis in 0.8% agarose gels. Plasmids with inserts of the appropriate size were selected for DNA sequence analysis. Inserts were sequenced in both directions by mixing 200-500 ng plasmid DNA with the either the 21M13 or M13rev sequencing primers (see Table I), and processed using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analyzed on an Applied Biosystems 3700 sequencer. Among the plasmids which contained the sequence shown at SEQ ID NO: 12 correctly inserted between the NdeI sites of pDEST12.2, miniprep #6 was selected for large-scale plasmid DNA preparation. A large-scale preparation of plasmid DNA was performed using the Qiagen ENDO-free Megaprep kit (cat no. 12381). The E. coli strain JM101, transformed with miniprep #6 above was grown overnight in 500 ml LB medium containing 100 μg/ml ampicillin and plasmid DNA was isolated from the saturated culture according to the protocol supplied by the manufacturers. The DNA concentration was adjusted to 5 mg/ml in preparation for the fast track electroporation protocol (see Example 11 below).

### Example 9

### Cloning of a Truncated Variant of EPO Encoded by Exons 1, 2, and the First Two Amino Acids of Exon 3 of the Human Gene EPO in Which the Free Cysteine Residue at Position 7 of the Mature Protein is Replaced by Serine (Named Herein EPOv C34S and EPOvm C34S in its Mature Form)

One consequence of truncating full length Erythropoietin to generate the variant EPOvm is that cysteine residue at position 7 of the mature protein (position 34 of EPOv) which is normally paired in the full length molecule is left free and thus could be available for inter-molecular pairing. Thus in order to avoid possible problems of aggregation of this small peptide fragment, the derivative C34S was constructed by oligonucleotide-directed mutagenesis.

Two complementary mutagenesic PCR primers AS675 and AS676 (see table I) were designed and PCR reactions were performed as follows: In PCR reactions 1 and 2, the template DNA was EPOv cloned into pDEST12.2 expression vector (see example 8 here above). In reaction 1, primers AS671 and AS676 were used to amplify the N-terminal part of the sequence shown in FIG. 8; in reaction 2, primers AS675 and AS672 were used to amplify the C-terminal part of the sequence shown in FIG. 8. In both cases, reaction mixtures were set up containing 1×PCR buffer, 0.2 mM each dNTP, 0.5 mM each PCR primer, 50 ng template DNA, and the reaction was initiated by addition of 5U PfuTurbo (Stratagene). Cycling conditions were: 95° C. 2 min (1 cycle); 95° C. 15 sec, 50° C. 30 sec, 72° C. 70 sec (25 cycles); 75° C. 7 min (1 cycle). An aliquot of each PCR reaction was analysed by electrophoresis in 0.8% agarose gels to estimate the PCR efficiency and yield. In a third PCR reaction, equimolar amounts the overlapping products of reactions 1 and 2 were mixed together and amplified in the presence of PCR primers AS671 and AS672 to generate the full length 0.9 kb fragment. PCR reaction conditions were as described above for reactions 1 and 2. An aliquot of the purified PCR reaction was digested with NdeI (New England Biolabs) for 2 h at 37° C. using the enzyme buffer supplied by the manufacturer. In parallel, an appropriate amount of the pDEST12.2 expression vector was digested with NdeI. The digested vector and insert were each separated on a 0.8% agarose gel, the corresponding fragments were excised and purified using the Wizard Cleanup System (Promega) according to the protocol provided by the manufacturer. The purified vector DNA and PCR product were mixed in a molar ratio of 1:3 and precipitated overnight at −20° C. by addition of 2.5 volumes ethanol. The precipitated DNA was recovered by centrifugation, washed in 70% ethanol, dried under vacuum and ligated in a final volume of 10 μl using the Rapid Ligation Kit (Roche Diagnostics) according to the protocol supplied by the manufacturers.

The ligation mixture was then used to transform E. coli strain JM101 as follows: 50 μl aliquots of competent JM101 cells were thawed on ice and 1 μl or 5 μl of the ligation mixture reaction was added. The cells was incubated for 40 min on ice and then heat shocked by incubation at 42° C. for 2 min. 1 ml of warm (room temperature) L-Broth (LB) was added and samples were incubated for a further 1 h at 37° C. The transformation mixture was then plated on LB plates containing ampicillin (100 μg/ml) and incubated overnight at 37° C. Single colonies were picked for plasmid isolation.

Plasmid DNA preparation, restriction digestion and sequence analysis.

Miniprep plasmid DNA was prepared from 5 ml cultures using a Biorobot 8000 robotic system (Qiagen) or Wizard Plus SV Minipreps kit (Promega cat. no. 1460) according to the manufacturer's instructions. Plasmid DNA was eluted in 80 ml of sterile water. The DNA concentration was measured using an Eppendorf BO photometer or Spectramax 190 photometer (Molecular Devices).

Aliquots of miniprep plasmid DNAs (100-200 ng) were digested with NdeI for 2 h at 37° C. and analysed by electrophoresis in 0.8% agarose gels. Plasmids with inserts of the appropriate size were selected for DNA sequence analysis. Inserts were sequenced in both directions by mixing 200-500 ng plasmid DNA with the either the 21M13, M13rev or AS673 sequencing primers (see Table I) and processed using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analyzed on an Applied Biosystems 3700 sequencer. Among the plasmids which contained the sequence correctly inserted between the NdeI sites of pDEST12.2, miniprep #2 was selected for large-scale plasmid DNA preparation.

A large-scale preparation of plasmid DNA was performed using the Qiagen ENDO-free Megaprep kit (cat. no. 12381). The E. coli strain, JM101 transformed with miniprep #2 above were grown overnight in 500 ml LB medium containing 100 μg/ml ampicillin and plasmid DNA was isolated from the saturated culture according to the protocol supplied by the manufacturers. The DNA concentration was adjusted to 5 mg/ml in preparation for the fast track electroporation protocol (see Example 11 below).

TABLE I

| | |
|---|---|
| Primer | sequence |

| | | |
|---|---|---|
| AS671 | CAAGTGTATCATATGCCAAG | (SEQ ID NO: 24) |
| AS672 | CAAGCAGCAAGCATATGCAG | (SEQ ID NO: 25) |
| AS673 | CACGACGGGCCACCATCACCATCACCATTGAAACC | (SEQ ID NO: 26) |
| AS674 | GGTGGCCCGTCGTGATATTCTCG | (SEQ ID NO: 27) |
| AS675 | CCACGCCTCATCAGTGACAGCCGAG | (SEQ ID NO: 28) |
| AS676 | CTCGGCTGTCACTGATGAGGCGTGG | (SEQ ID NO: 29) |
| 21M13 | GTAAAACGACGGCCAGT | (SEQ ID NO: 30) |
| M13rev | CAGGAAACAGCTATGACC | (SEQ ID NO: 31) |

### Example 10

### Biological Activity of the EPO Polypeptides and Variants of the Present Invention

The biological activity of the polypeptides of the present invention can be verified using several biological assays that are known per se in the art.

10.1 Binding of the Polypeptides of the Present Invention to the Erythropoietin-Receptor in an in Vitro Cellular Test:

Scatchard analyses of EPO binding to its receptor have been well described in the art, for example in Nagao M, et al:, Blood. 1993;81(10):2503-10.

The binding of radioiodinate (¹²⁵I) polypeptide to erythropoietin-receptor expressing cells is assayed. BHK-21 cells (accessible at the American Type Culture Collection (ATCC) (Manassas, Va., USA), under the reference number CCL-10) are inoculated in tissue culture plates and cultured for 48 hours. The culture medium in each well is then replaced with a binding mixture consisting of the fresh culture medium, and of ¹²⁵I-polypeptide with or without different concentration of unlabeled polypeptide. After incubation at 10° C., each well is washed with ice-cold phosphate-buffered saline (PBS). To each well, trypsin is added to detach the cells. The radioactivity of the cell suspension is counted. The number of cells per well is also counted. Specific binding at a given ¹²⁵I-polypeptide concentration is defined as the difference in bound radioactivity between samples incubated in the absence of unlabeled EPO or in the presence of an excess of unlabeled polypeptide. Scatchard plot analyses of the binding data are performed.

10.2 Induction of Tyrosine Phosphorylation of JAK2 by the Polypeptides of the Present Invention in Erythropoietin-Receptor Expressing Cells:

Different tests are described in the art to test the tyrosine phosphorylation of JAK2 and these techniques are known to those skilled in the art. For example, the induction of Tyrosine phosphorylation of JAK2 by the polypeptides of the present invention in erythropoietin-receptor in expressing cells can be tested by the test disclosed by Witthuhn B A et al. in Cell. 1993; 74(2):227-36.

10.3 Stimulation of Proliferation of Erythropoietin-Receptor (EpoR) Expressing Cells by the Polypeptides of the Present Invention:

10.3.1 Cell-Based Proliferation Assay Using the Human Erythroleukemia Cell Line TF-1:

For this purpose, small quantity of the polypeptides to be tested is generated according for example to the method described in Examples 2, 4 or 6. The protein is then tested for its ability to induce a biological response using the human erythroleukemia cell line TF-1 that expresses the erythropoietin-receptor and is dependant on either IL-3, GM-CSF or EPO for its growth (Hammerling, U., et al., J. Pharma. Biomed. Anal. 12 :1455-69 (1996)). A cell-based proliferation assay using this factor-dependant cell line is used as the industry standard for measurement of in vitro biological activity of erythropoietin (Kitamura, T., et al., J: Cell Physiol. 140: 323(1989)). This assay is very sensitive and can detect very small amounts of biologically active protein. An additional advantage of this assay is that unpurified supernantants from cell cultures expressing modified erythropoietin molecules of the present invention may be used for testing biological activity rather than employing extensive purification methods to obtain pure protein. The activity of each of the analog proteins will be compared to that of wild type protein and necessary quantification can be done using commercially available ELISA kits.

10.3.2 Cell-Based Proliferation Assay Using the Ba/F3-EpoR Cell Line:

The biological activity of the polypeptides of the present invention can be evaluated by measuring the proliferation of Ba/F3-EpoR cells.

For this purpose, a DNA fragment corresponding to the entire coding sequence of human EPO receptor (EpoR) is obtained by PCR and cloned into an expressing vector containing the thymidine kinase (tk)-neo marker. After linearization by digestion with restriction enzymes, the vector construct is introduced into Ba/F3 (IL-3 dependent murine pro B cell line established from peripheral blood; accessible at the Deutsche Sammlung von Mikroorganismen und Zellkulturen GmbH (DSMZ, Braunschweig, GERMANY), under the reference number ACC 300). Neomycin resistant cells, expressing the EpoR construct, are selected in 2 mg/ml G418, and individual clones are obtained by limiting dilutions.

Clones expressing EpoR are analyzed for their responses to recombinant human EPO (rhEPO) (Amgen) or the polypeptides of the present invention to be tested, in a biological assay measuring stimulation of proliferation of Ba/F3-EpoR cells. Stimulation of proliferation of Ba/F3-EpoR cells in response to rhepo and the polypeptides of the present invention to be tested, respectively, are measured by the extent of incorporation of [³H]-thymidine into the cellular DNA. Cells are initially starved of IL-3 for 16 hours, and subsequently, seeded in 96 well plates at a density of 25,000 cells/well in media containing rhepo or the polypeptides of the present invention to be tested, at various concentrations. After incubation for 22 hours, 1 μCi of [³H]-thymidine/well is added to the wells, and the cells are incubated for an additional 6 hours before being harvested. Cell-incorporated radioactivity is determined in the presence of 40 μl of scintillation fluid (for example icroscint 20) using for example a Top Count Counter (Packard Instruments). The efficiency of the polypeptides of the present invention in stimulating incorporation of tritiated thymidine in Ba/F3-EpoR cells can be compared to the efficiency of stimulation by rhepo.

10.3.3 Schwann Cells Proliferation Assays:

Li et al (Li X, Gonias S L, Campana W M. Glia. Vol 51(4):254-65) have shown that Schwann cells express Epo receptor. The biological activity of the polypeptides of the present invention can be evaluated by determining BrdU incorporation as described here below.

Primary Schwann Cell Cultures

Schwann cells are isolated from sciatic nerves of 1-day-old Sprague-Dawley rats as described by Hiraiwa et al. (Hiraiwa M et al. 1997. Proc Natl Acad Sci USA 94:4778-4781) and Campana et al. (Campana W M, et al, 1998. FASEB J 12:307-314). Schwann cells are further separated from fibroblasts using anti-fibronectin antibody and rabbit complement. This results in approximately 99% pure Schwann cell cultures as can be assessed by S-100 immunofluorescence. Primary Schwann cells are maintained in DMEM containing 10% fetal bovine serum (FBS), 100 U/ml penicillin, 100 mg/ml streptomycin, 21 mg/ml bovine pituitary extract and 4 mM forskolin (complete medium) and incubated at 37° C. under humidified 5.0% CO2. Schwann cells are expanded by passing the cells 3-4 times after the cultures are established.

Schwann Cell Proliferation Assays

Schwann cells are plated at 5,000 cells per well in 96-well plates in complete medium. Cells are allowed to attach overnight and are then washed, and subsequently cultured in DMEM with 1% FBS at 37° C. with or without rhepo or the polypeptides of the present invention to be tested for 24 h. Complete media (containing bovine pituitary extract, BPE) is used as a proliferative control. BrdU incorporation is then measured, as an index of DNA synthesis during S-phase using the Cell Proliferation ELISA kit (Roche Applied Science, Indianapolis, Ind.). BrdU is added for the last 20 h of the 24-h rhepo or the polypeptides of the present invention to be tested treatment period.

10.4 Stimulation of Red Blood Cell Production (In Vivo Activity of the Polypeptides of the Present Invention Determined by the Normocythaemic Mouse Assay):

The normocythaemic mouse bioassay is known in the art (Pharm. Europa Spec. Issue Erythropoietin BRP Bio 1997 (2)) and a method in the monography of erythropoietin of Ph. Eur. BRP. Normal healthy mice, 7-15 weeks old, are administered s.c. 0.2 ml of the the polypeptides to be tested in BSA-PBS solution or buffer as control. Over a period of 6 days, blood is drawn by puncture of the tail vein and diluted such that 1 μl of blood is present in 1 ml of an 0.15 μmol acridine orange staining solution. The staining time is 3 to 10 minutes. The reticulocyte counts are carried out microfluorometrically in a flow cytometer by analysis of the red fluorescence histogram. The reticulocyte counts are given in terms of absolute figures (per 30,000 blood cells analyzed).

The results give an indication of the in vivo activity of the polypeptides of the present invention on their capability to increase the amount of reticulocytes.

10.5 Induction of Proliferation of Erythroid Cells by the Polypeptides of the Present Invention:

The biological activity of the polypeptides of the present invention can be evaluated by measuring the ability of the polypeptides of the present invention to stimulate the production of erythroid colonies from human bone marrow cells.

Fresh human bone marrow aspirates are obtained from healthy donors. The mononuclear fraction is enriched for CD34 by immunomagnetic positive selection. Methylcellulose cultures are initiated with 1000 cells in complete methylcellulose media without erythropoietin (Stem Cell Technologies, Vancouver, BC). Culture medium is later supplemented with 50 ng/mL rhepo or the polypeptides of the present invention to be tested and 50 ng/ml of kit ligand (KL), which acts as a stem cell factor, and synergizes in these assays with Epo to promote the formation of erythroid colonies. After 12-14 days, colonies are enumerated and phenotyped on an inverted light microscope. The efficiency of the polypeptides of the present invention in stimulating production of erythroid colonies from human bone marrow cells is measured and compared to the results from assays with rhEPO.

10.6 Induction of Maturation of Erythroid Cells by the Polypeptides of the Present Invention:

The biological activity of the polypeptides of the present invention can be evaluated by measuring the ability of the polypeptides of the present invention to stimulate the formation of immature and mature erythroid cells in liquid cultures of bone marrow cells. Twenty thousand CD34+ cells isolated as described above are cultured in IMDM/10% FCS in the presence of CF, 50 ng/ml of kit ligand (KL) and either 50 ng/mL rhepo (Amgen) or the polypeptides of the present invention to be tested. 7-10 inch plates of culture cells are counted by a hematocytometer and subsequently assayed for expression of the erythroid cell surface markers CD36, CD71 and Glycophorin A. The results permit to determine the ability of the polypeptides of the present invention in stimulating the formation of immature and mature erythroid cells in bone marrow liquid culture.

10.7 Vasoactive Action of the Polypeptides of the Present Invention:

The biological activity of the polypeptides of the present invention can be tested by measuring the blood pressure of a mammal treated with polypeptides of the present invention compared to non-treated subjects. Such mammals can for example be mice, rats, rabbits, dogs, bovine or monkeys (for example Chimpanzee). Those skilled in the art are well aware of the different method to measure blood pressure in these mammals.

10.8 Effect of the Polypeptides of the Present Invention on the Hematocrit Level:

The biological activity of the polypeptides of the present invention can be tested by measuring the hematocrit level of a mammal treated with polypeptides of the present invention compared to non-treated subjects. Such mammals can for example be mice, rats, rabbits, dogs, bovine or monkeys (for example Chimpanzee).

Hematocrit is a measurement of red blood cells, and is commonly expressed as the percentage of total blood volume which consists of erythrocytes. Several tests have been described in the art to measure hematocrit and are well known to those skilled in the art. For example, retro-orbital bleeding is performed using specific heparin capillaries with calibrated diameter. Capillaries are centrifuged 2 minutes at room temperature in a micro-hematocrit centrifuge (StatSpin—IRIS Company). Peripheral blood samples are collected at day 0,3,5,7,10 and 12. To measure hematocrit, the ratio (%) of red blood cells volume versus total blood volume is calculated.

10.9 Neuro-Protection Activity of the Polypeptides of the Present Invention:

10.9.1 In Vivo Test of the Neuro-Protection Activity:

The neuroprotection activity of the polypeptides of the present invention can be tested for example in a middle-cerebral artery occlusion model. Sprague-Dawley male rats weighting 250 g are subjected to middle-cerebral artery occlusion consisting of a small core lesion within a much larger penumbra produced by 60 min of reversible ischemia, as described by Morishita, E., et al, 1997, Neuroscience 76, 105-116. The animals receive either, the polypeptide of the present invention to be tested, recombinant wild type human EPO (rhEPO) as a positive control (5,000 units/kg of bodyweight) or saline as a negative control, i.p. at the time of occlusion. The brain is removed after 24 h, serially sectioned (50 mm thick), blocked in 3% H2O2 in methanol for 10 min, permeabilized for 2 min in 0.1% Triton X-100/sodium citrate at 4° C., and treated with TUNEL reaction mixture according to the manufacturer's protocol (In Situ Cell Death Detection Kit, Roche Diagnostics). Positive neurons are identified after development (30 min in diaminobenzidine, dehydrated, and cover slipped). As a negative control, terminal transferase is omitted.

Siren A L, et al., (Proc Natl Acad Sci USA. 2001, 98(7):4044-9) and Brines M L et al., (Proc Natl Acad Sci USA. 2000; 97(19):10526-31) have shown that rhEPO has a neuronal protection activity in such a model.

10.9.2 In Vivo Test of the Neuro-Protection Activity in an EAE Model:

The utility of the polypeptides of the present invention in treating demyelinating diseases, e.g. multiple sclerosis (MS) or Guillain-Barre syndrome as hereinabove specified, may be demonstrated in animal test methods, for example in accordance with the methods hereinafter described. The most widely used animal model for multiple sclerosis is Experimental Autoimmune Encephalomyelitis (EAE), based on shared histopathological and clinical features with the human disease:

The chronic EAE model in C57B1/6 mice shares some common traits with the primary progressive (PP) or secondary progressive (SP) forms of MS. Mice are immunized in both flanks at day 0 and day 7 with 200 μg s.c. of myelin oligodendrocyte glycoprotein (MOG) in Complete Freund's Adjuvant (CFA) and followed by two injections (on day 0 and day 2) with 500 ng i.p. of B. pertussis toxin.

Groups are composed of 10 to 13 EAE mice. Clinical scores, overall health status, body weight and mortality are recorded daily. Starting from day 7 the animals are individually examined for the presence of paralysis by means of a clinical score: 0=no sign of disease, 1=tail paralysis, 2=tail paralysis+hindlimb weakness or partial hindlimb paralysis, 3=tail paralysis+complete hindlimb paralysis, 4=tail paralysis+hindlimb paralysis+weakness or partial paralysis of forelimbs, 5=moribund or dead.

Starting from day 10-12, most animals are becoming increasingly paralysed. The pathology is chronic and animals do not show signs of remissions after the first clinical signs of disabilities, and during the following 28 to 30 days of observation.

Therapeutic treatments are started at the onset of the disease, thus once the disease is already established but still progressing and continued for 28 to 30 days. Subcutaneous daily treatment with mIFNβ (Serono Pharmaceutical Research Institute, Geneva) at the dose of 20,000 U/mouse shows beneficial effects on clinical output by significantly reducing the severity of the disease from complete hindlimb to partial hindlimb paralysis. The polypeptides of the present invention can be tested for their activity in this model by daily double treatment of the mice with different doses of the polypeptide to be tested. Control vehicle-treated EAE groups following the same administration routes are included in experiments.

10.9.3 In Vitro Test of the Neuro-Protection Activity:

The neuroprotection activity of the polypeptides of the present invention can be tested in vitro for example by using primary motoneurons in culture as described by Siren A L, et al., (Proc Natl Acad Sci USA. 2001, 98(7):4044-9). Spinal cords are obtained from 15-days old Sprague-Dawley rat embryos. The ventral horn is trypsinized and centrifuged through a 4% BSA cushion for 10 min at 300×g. Cells (representing mixed neuronyglia culture) are seeded at a density of 2,000 cells/cm2 into 24-mm well plates precoated with poly-DL-ornithine and laminin. Motoneurons are further purified by immunopanning (as described by Mettling, C., et al., 1995, J. Neurosci. 15, 3128-3137) and the cells are seeded at low density (20,000 cells/cm2) onto 24-mm well plates precoated with poly-DL-ornithine and laminin, and containing complete culture medium (Neurobasaly/B27 (2%)/0.5 mM L-glutamine/2% horse serum/25 μM 2-mercaptoethanol/25 μM glutamate/1% penicillin and streptomycin/1 ng/ml BDNF). This medium (without glutamate) is readded to cultures on days 4 and 6. Cell death is induced on day 6 in culture by 48 h serum/BDNF deprivation or by incubation for 48 h with kainic acid (5 μM for mixed neuron/glia cultures; 50 μM for purified cultures). The polypeptide of the present invention to be tested, or rhEPO as a positive control (10 units/ml) or vehicle as a negative control is added to the cultures 72 h before induction of cell death, and treatment continued for 48 h. The medium is then discarded and the cells fixed with 4% (vol/vol) paraformaldehyde in PBS for 40 min, permeabilized with 0.2% Triton X-100, blocked with 10% (vol/vol) FCS in PBS, incubated with antibodies against nonphosphorylated neurofilaments (SMI-32; 1:9,000) overnight, and visualized by using the avidin-biotin method with diaminobenzidine. Viability of motoneurons is assessed morphologically by counting SMI-32 positive cells across four sides of the cover slip. Staining for apoptotic bodies is done by using H33258 (as described by Galli, G. & Fratelli, M. (1993) Exp. Cell Res. 204, 54-60.).

Siren A L, et al., (Proc Natl Acad Sci USA. 2001, 98(7):4044-9) have shown that rhEPO has a neuroprotective activity in such a model.

10.10 Cardio-Protection Activity in Vitro and/or in Vivo of the Polypeptides of the Present Invention:

Different tests are described in the art to test the cardioprotection activity of a polypeptide both in vitro and in vivo these techniques are known to those skilled in the art. Example of such techniques are described here below and in the literature cited which is incorporated by reference.

10.10.1 Enhancement of the Survival of Cardiomyocyte Cells in Vitro by the Polypeptides of the Present Invention:

The cardio-protection activity of the polypeptides of the present invention can be tested for example in the following model. Left ventricular cardiomyocytes are isolated from 3-month-old male Sprague-Dawley rats as described (Fiordaliso, F., et al., (2001) Diabetes 50, 2363-2375 and Leri, A., et al., (1998) J. Clin. Invest. 101, 1326-1342). Briefly, under chloral hydrate anesthesia (300 mg/kg of body weight), hearts are excised, and the myocytes are dissociated by collagenase. Cardiomyocytes (98-99% pure) are plated onto Petri dishes coated with 0.5 μg/cm² laminin at a density of 2×10⁴ cells per cm2. Cells are incubated in serum-free medium consisting of modified Eagle's medium (MEM) with nonessential amino acids, transferrin (10 μg/ml), BSA (0.1%), and antibiotics. To remove unattached myocytes, this medium is exchanged for new medium 30 min after plating. Hypoxic conditions are generated by exposure in an air-tight chamber flushed continuously with nitrogen and maintained for 28 h. This procedure reduced oxygen tension in the medium to 5 mmHg (≈3% normal; 1 mmHg=133 Pa). Hypoxia is maintained for 28 h. The polypeptide of the present invention to be tested or rhEPO (100 ng/ml) as a positive control, Hepes (20 mM), or both are added to the medium 30 min before the induction of hypoxia. Hepes is used to correct the acidosis produced by prolonged hypoxia.

Cellular necrosis is quantified by using two independent methods (ethidium monoazide bromide; Molecular Probes) and hairpin oligonucleotide probe with blunt ends (hairpin 2; Synthetic Genetics, San Diego). Apoptosis is assessed by use of a hairpin oligonucleotide probe with single-base 3′ overhangs (hairpin 1; Synthetic Genetics) and a terminal deoxynucleotidyltransferase (TdT) assay (Cigola, E. et al., (1997) Exp. Cell Res. 231, 363-371.). The number of myocytes measured in each preparation is of a minimum of 300.

Calvillo L, et al., (Proc Natl Acad Sci USA. 2003; 100(8):4802-6) have shown that rhEPO has a cardioprotective activity in such a model.

Parsa C J et al. (J Clin Invest. 2003. 112(7):999-1007) and Moon C et al. (Proc Natl Acad Sci USA. 2003;100(20):11612-7) have also shown a cardioprotection activity of rhEPO in vitro using other models. These models can also be used to test the cardio-protection activity of the polypeptides of the present invention.

10.10.2 Enhancement of the Survival of Cardiomyocyte Cells in Vivo by the Polypeptides of the Present Invention:

Male Sprague-Dawley rats (about 250 g) are anesthetized with chloral hydrate (150 mg/kg i.p.) and diethyl ether and are ventilated (61 breaths per min, tidal volume 1.2 ml/100 g of body weight) through an endotracheal cannula. The left anterior descending coronary artery (LAD) is ligated with a 5-S silk suture after exteriorization of the heart through a 15-mm opening at the fifth intercostal space. A plain knot is tied over two pieces of suture, which is removed after 30 min to initiate reperfusion. The thorax is closed under negative pressure, and the rat is weaned from mechanical ventilation under continuous electrocardiographic monitoring. Ischemia is confirmed by the appearance of ventricular ectopy and blanching of the myocardium. Successful reperfusion is indicated by a restoration of normal rubor. Sham-operated rats undergo identical surgical procedures, but without ligation of the LAD. The polypeptide of the present invention to be tested or rhEPO (5,000 units/kg of body weight) as a positive control is administered i.p., either before the induction of ischemia (pretreatment) or at reperfusion (posttreatment). Each animal receive additional dose of the polypeptide of the present invention to be tested or rhEPO daily until study completion.

Rats surviving a 30-min LAD occlusion for 7 days undergo hemodynamic evaluation followed by fixation perfusion of the heart for histomorphometry as described by Calvillo L, et al., (Proc Natl Acad Sci USA. 2003; 100(8):4802-6).

Parsa C J et al. (J Clin Invest. 2003. 112(7):999-1007) and Moon C et al. (Proc Natl Acad Sci USA. 2003;100(20):11612-7) have also shown a cardioprotection activity of rhEPO in vivo using other models. These models can also be used to test the cardio-protection activity of the polypeptides of the present invention.

10.11 Stimulation of the Proliferation of Cancer Cells by the Polypeptides of the Present Invention:

In vitro and in vivo models to test the ability of polypeptides to stimulate the proliferation of cancer cells are well known in the art. For example the proliferation of one or more cell line(s) derived from the cancer to be tested can be measured in vitro by using different techniques well known in the art. Such techniques include for example incorporation of [³H]-thymidine, MTT cell proliferation assay, counting of cell numbers . . . .

In vivo models include for example human tumors cells xenografted onto athymic mice. These models can also be used to test anti-tumorigenic activity of the antibodies of the present invention.

### Example 11

### Neuroprotective Activity of EPOv1, EPOv2, EPOv3 and EPOv of the Present Invention

The neuroprotective effect of the Epo variants was tested using the murine sciatic nerve crush model following protein delivery by ‘Fast Track’. The cDNAs encoding EPOv1, EPOv2, EPOv3 and EPOv were cloned or subcloned in the expression vector pDEST12.12. In the Fast Track procedure, cDNAs cloned into the expression vector pDEST12.12 are electroporated into the muscle of recipient mice and the encoded protein is expressed by the cells of the muscle and secreted into the circulation of the host mouse. In these experiments each cDNA was electroporated into 6 mice to provide statistical significance of the observed effects. Following electroporation, the effects on the red blood cell volume (haematocrit) and the compound muscle action potential (CMAP) measured in the gastrocnemius muscle, were monitored.

Electroporation of Epo Variant cDNAs into Muscle for Sciatic Nerve Crush

In the original Fast Track protocol the gastrocnemius muscle was used as the site for electroporating cDNA. However, since in the sciatic nerve crush model, the electromyographic readout (EMG) is also performed using the gastrocnemius muscle we have tested other sites of electroporation in order not to interfere with the electrophysiological readout. As a result of these tests, the muscles of the upper forelimb were selected and used in the experiments described herein.

On day 0, 4 days before the nerve crush, groups of 6 female C57BL/6 mice, (Elevage Janvier) were anaesthetized with isofluran (isofluran Baxter, Ref: ZDG9623), and a first electroporation into the right fore-limb was performed as follows: cDNA was prepared at 2 mg/ml in 0.9% NaCl, 6 mg/ml L-Glutamate (Sigma Ref: P4761). Before electroporation, 25 μl of hyaluranidase (100 U/ml) was injected into the muscle, followed 20 minutes later by 25 μl (50 mg) cDNA. An echographic gel was applied, and the muscle was held between 2 circular electrodes (0.5 mm diameter) of the ElectroSquarePorator BTX (Ref.: ECM830). An electric field of 75 Volts was applied for 20 ms, and this was repeated 10 times with an interval of 1 second between each pulse.

On day 12 of the experiment, 1 day after the first EMG readout, a second electroporation was performed as described above into the left fore-limb. A second EMG readout was performed on day 18 of the experiment.

Sciatic Nerve Crush in Mice

Female mice (C57BL/6 mice, Elevage Janvier) were anaesthetized with isoflurane and the body temperature was checked. The right sciatic nerve was crushed using a Kocher clamp (2×30 sec). Electromyographic parameters, namely amplitude, latency and duration, were evaluated 7 and 14 days after the crush using a Medtronic apparatus (Keypoint model). Compound muscle action potential (CMAP) was measured in the gastrocnemius muscle after a single 0.2 ms stimulation of the sciatic nerve at a supramaximal intensity (12.8 mA). The amplitude (mV) is related to the number of active motor units and is influenced by the axonal degeneration. The latency (ms) is related to the motor nerve conduction and neuromuscular transmission velocities and is influenced by demyelination. The duration (time needed for one depolarization/repolarization cycle) is a qualitative index of conduction and is also influenced by demyelination.

Results of the Nerve Crush Experiments Following Fast Track Delivery

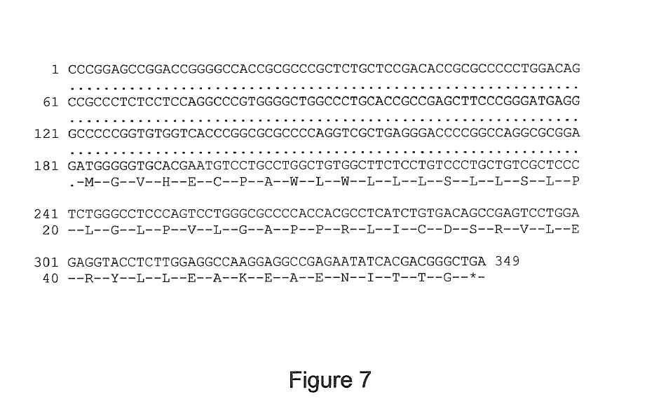

The CMAP parameter most amenable to improvement by erythropoietin and the EPO variants is the latency. In the experiments shown in FIG. 9, all EPO variants show statistically significant improvement in latency comparable to the activity of the wild type protein. On the other hand, neither the vector alone nor the expression of an irrelevant protein, IL4, shows any positive effects on latency. Interestingly, Epov which expresses just the N-terminal 28 amino acids of erythropoietin common to all variants, is as active as the wild type protein, suggesting that all neuroprotective activity is encompassed within this sequence (see FIG. 7 and SEQ ID NO: 13).

EPOv C34S was also tested by Fast Track delivery and had an activity comparable to EPOv as tested at day 7 following nerve crush.

### Example 12

### Effect of EPOv1, EPOv2, EPOv3 and EPOv of the Present Invention on the Hematocrit Level

The hematocrit level of the mouse treated according to the Fast Track Protocol described at example 11 was measured.

Hematocrit Readout:

A blood sample is transferred to a heparinized glass capillary tube, and centrifuged in CritSpin centrifuge at 16000 rpm for 120 sec. The hematocrit reading is taken as the sedimented red blood cell volume expressed as a percentage of the total volume of the sample.

Results of the Haematocrit Experiments:

From the results shown in FIG. 10, we conclude that only wild type erythropoietin is able to activate the haematopoietic pathway while none of the Epo variants (EPOv1, EPOv2, EPOv3 and EPOv) have an effect on the red blood cell count.

### Example 13

### Cell-Based Proliferation Assay Using the Human Erythroleukemia Cell Line TF-1

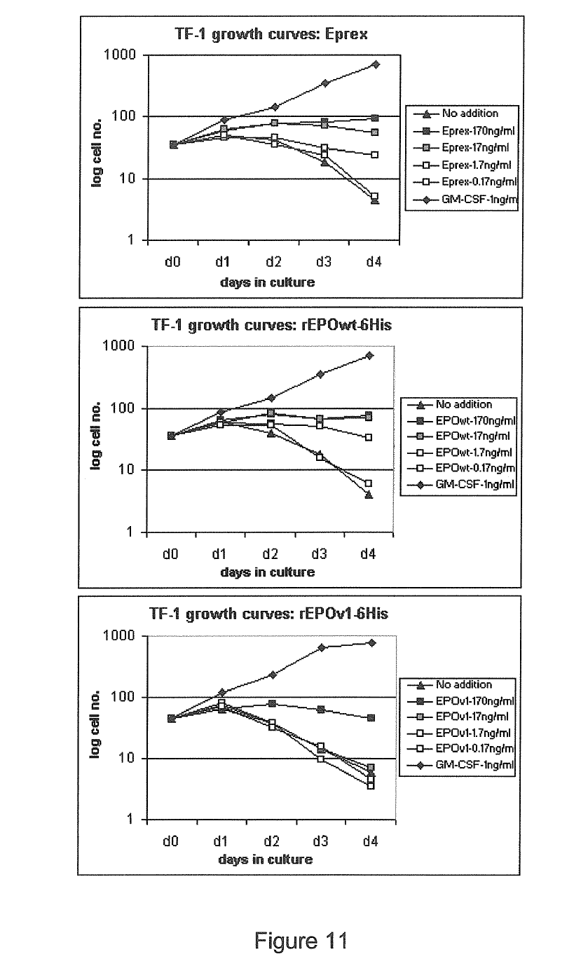

TF-1 (ATCC #CRL-2003) is an erythroleukemia cell line known to be dependent on the presence of cytokines such as GM-CSF for survival and proliferation. Tests were conducted to investigate the effects of erythropoietin on TF-1 cell growth. In FIG. 11 the effects of rEPOwtm (recombinant wild type EPO His-tagged obtained according to a protocol similar to example 2) and rEPOv1m (recombinant EPOv1m His-tagged obtained as described at example 2) were compared to the activity of the commercially available product, Eprex. The results show that while GM-CSF at 1 ng/ml is able to support cell proliferation with a culture doubling time of approximately 20 hr, the commercially available erythropoietin (Eprex1000) shows dose dependent survival of TF-1 cells at concentrations above 0.2 U/ml (1,7 ng/ml), but no proliferation (top panel). The recombinant EPOwtm-6His protein shows an activity identical to that of Eprex (middle panel). On the other hand EPOv1m shows partial protection at 170 ng/ml but no protection at lower doses (bottom panel). We conclude that the activity of EPOv1m assayed in this erythroid cell line is approximately 1% of the activity of the full length EPOwtm. This reduced activity is consistent with the in vivo data showing loss of activity in the haematocrit assay (FIG. 10).

### Example 14

### Neuroprotective Activity of EPOv1 and EPOv of the Present Invention

The effects of subcutaneous injection of EPOwt and EPO variant proteins on recovery from sciatic nerve crush was studied.

EPOv1m-6His was produced in HEK293 cells as described in Example 2. EPOwtm-6His was produced using the same method. EPOvm_C34S-6His peptide was synthesized chemically (Eurogentec). EPOvm_C34S-6His corresponds to EPOvm C34S (amino acids 28 to 55 of SEQ ID NO: 15) linked to a 6His tag. A “shuffled” peptide (same 28 amino acids of EPOvm C34S but in a random order) was also synthesized chemically (Eurogentec).

Equimolar amounts of protein, equivalent to 50 μg/Kg (6000 U/Kg) Eprex were diluted in PBS to give a final volume of 10 μl per gram mouse body weight and injected into upper back muscle. Injections were repeated once daily, 5 days per week for the duration of the experiment. The effects of treatment on the compound muscle action potential (CMAP) measured in the gastrocnemius muscle, were monitored on days 8 and 15 following nerve crush.

Results of the Nerve Crush Experiments Following Delivery of Proteins by Injection

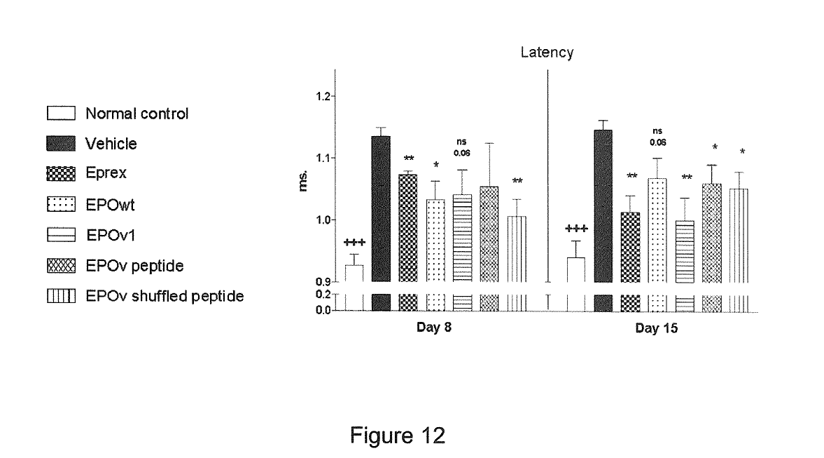

As shown in FIG. 12, injection of purified EPOv1 and EPOv both show an activity comparable to the wild type protein for the latency parameter while no effect was observed following injection of vehicle alone. As found previously in the Fast Track experiments, the effects on duration and amplitude are less pronounced.

The “shuffled” peptide was also tested for activity. This peptide showed activity in this experiment. Without wishing to be limited by speculation, this may be due to either retention of a functional motif, a contamination or an experimental error. Further experiment may be needed to elucidate the observed activity of this “shuffled” peptide.

### Example 15

### Effects of EPOwt and EPO Variant Proteins on MBP Content of Sciatic Nerves Following Crush

Since the main observed effects of EPO and EPO variants on CMAP are on the latency, which is influenced in large part by the degree of myelination, we sought to correlate the electrophysiological readout with biochemical changes taking place in the regenerating myelin sheath. To do this, a novel procedure to determine the level of myelin basic protein (MBP) in sciatic nerves was developed.

Following dissection of the sciatic nerve, the crushed section can be easily identified as thicker scarred tissue which we decided to avoid for the measurement of MBP. For each animal therefore a section of approximately 2 mm of the crushed nerve distal to the crush site was removed, together with the corresponding section of the contralateral nerve. The dissected nerve sections were transferred to a 96-well round-bottom cell culture dish maintained on dry ice. For extraction, 20 μl 10% SDS was added and the plates were sonicated in the X2020 microtiter plate sonicator (Misonix Inc) using 4×10 second pulses with the power setting at level 5. Each sample was then further extracted by addition of 180 μl Trizol (Invitrogen) and incubation at room temperature for 1 h. The microtitre plate was re-sonicated as above at power setting 7, and the contents of each well were transferred to an Eppendorf tube. The tubes were vortexed vigorously after addition of 50 μl chloroform, and phases were separated by centrifugation for 10 m at 14 Krpm. The lower organic phase was carefully removed without disturbing any pelleted insoluble material, and the proteins were precipitated by addition of 5 volumes acetone and incubation for 1 h at room temperature. Precipitated proteins were collected by centrifugation, washed in 70% ethanol, and dried briefly by centrifugation under vacuum. Pellets were solubilized by incubation in 0.2% SDS, 140 mM NaCl, 50 mM Tris pH8.0 at 4° C. overnight followed by 95° C. for 10 m. A further 50 μl 2% NP40, 1% deoxycholate, 140 mM NaCl, 50 mM Tris pH8.0 was then added to reconstitute triple detergent buffer used for the MBP Elisa.

Using this procedure, on day 16 of the experiment described in Example 14 after completion the second CMAP measurements, proteins were extracted from the sciatic nerves of both limbs of all 36 animals.

MBP Elisa

Protein concentrations in each sample were determined using the BCA protein assay (Pierce) according to the procedure recommended by the manufacturer. All samples were diluted to the same protein concentration and the MBP content of serial dilutions of each sample was determined by Enzyme-Linked ImmunoSorbent Assay (ELISA). For the Elisa, 96-well plates (Nunc Immunoplate, #439545) were coated overnight with antiMBP monoclonal antibody (Mab382, Chemicon) diluted 1:5000 in PBS. Plates were emptied and blocked with 0.1% BSA (Sigma, A9647) in PBS. Serial dilutions of the nerve extracts in Triple Detergent buffer (1% NP40, 0.5% deoxycholate, 0.1% SDS, 140 mM NaCl. 50 mM Tris pH 8.0) were added to the coated wells and incubated on a rocker platform for 2 h at room temperature. Serial dilutions of purified mouse MBP (Invitrogen, 13228-010) were included in the plate as calibration standard. After incubation, the wells were washed 3 times with PBS containing 0.5% Tween 20, and the detection antibody, rabbit antiMBP polyclonal antibody (Zymed 18-0038) diluted 1:300 in PBS, 0.1% BSA, was added and incubated for 2h on the rocker platform. The detection antibody was removed, and wells were again washed in PBS, Tween-20 as above. A biotinylated goat anti-rabbit polyclonal (Vector, BA-1000) diluted 1:10,000 in PBS 0.1% BSA was added for 1 h, wells were washed as above, and streptavidin-HRP (Amersham, RPN1051V) diluted 1:8000 was added. After 1 h, wells were washed 3 times in PBS, Tween-20 as above and the signal was developed by incubation for 30 min at room temperature with Sigmafast OPD (Sigma, P9187). At the end of this time, the reaction was stopped by addition of an equal volume of 2M H2SO4, and the plates were read at 492 nm.

The quantity MBP content of each sample was determined by reference to the MBP standard curve and expressed as ngMBP/μg total protein. The values obtained for crushed nerves were normalized to the values obtained for the corresponding contralateral nerves. Statistical analysis was performed using the ANOVA test.

Results on MBP Content of Crushed Sciatic Nerve Treated with EPOwt and EPO Variants.

The MBP content of sciatic nerves determined as described above was highly reproducible as judged by the small standard deviations within each group. In normal vehicle-treated sciatic nerves, MBP represents approximately 5% total extracted protein while in the distal section of crushed, vehicle-treated sciatic nerves 16 days post-crush, MBP represents approximately 1% total extracted protein. Groups treated with EPOwtm, EPOv1m or EPOvm C34S all show an increased level of MBP in the crushed nerve compared to the vehicle-treated group. We conclude that EPOwt and the EPO variants described in the present invention are able to stimulate MBP production, probably reflecting improved myelin regeneration following sciatic nerve crush. The electrophysiological consequences of EPOwt and the EPO variants as determined by the CMAP readout, thus correlate with an increased MBP and thus probably myelin content.

The “shuffled” peptide also showed activity in this experiment. Without wishing to be limited by speculation, this may be due to either retention of a functional motif, a contamination or an experimental error. Further experiment may be needed to elucidate the observed activity of this “shuffled” peptide.

1-43. (canceled)

44. An isolated polypeptide selected from the group consisting of:
(a) a polypeptide comprising an amino acid sequence differing from SEQ ID NO: 3 by the lack of at least one of the amino acids at positions 56 to 193;
(b) a polypeptide comprising an amino acid sequence differing from SEQ ID NO: 3 by the lack of amino acids 1 to 27 and the lack of at least one of the amino acids at positions 56 to 193;
(c) a polypeptide comprising SEQ ID NO: 13;
(d) a polypeptide comprising amino acids 28 to 55 of SEQ ID NO: 13;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations selected from the group consisting of: I33A, C34S, C34A, R37I, VI38S, I39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T1331, T134A, T134L, L135K, L135A, L135S, K143A, S153A, T159A, 1160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3; and
(g) a polypeptide differing from a), b), c), d) e) or f) by including at least one additional N-linked glycosylation site at position 84, 96, 113, 115, 116 or 141, said position being defined by reference to the position of the amino acid in SEQ ID NO: 3.

45. The isolated polypeptide according to claim 44, wherein said polypeptide is selected from the group consisting of:
(a) a polypeptide differing from SEQ ID NO: 3 by the lack of at least one of the amino acids at positions 56 to 193;
(b) a polypeptide differing from SEQ ID NO: 3 by the lack of amino acids 1 to 27 and the lack of at least one of the amino acids at positions 56 to 193;
(c) a polypeptide consisting of SEQ ID NO: 13;
(d) a polypeptide consisting of amino acids 28 to 55 of SEQ ID NO: 13;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N 51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3;
(g) a polypeptide differing from a), b), c), d) e) or f) by including at least one additional N-linked glycosylation site at position 84, 96, 113, 115, 116 or 141, said position being defined by reference to the position of the amino acid in SEQ ID NO: 3;
(h) a polypeptide comprising a polypeptide having at least 80% amino acid sequence identity with the polypeptide of a), b), c), d), e), f) or g); and
(i) a polypeptide having at least 80% amino acid sequence identity with the polypeptide of a), b), c), d), e), f) or g).

46. The isolated polypeptide according to claim 44, wherein said polypeptide is selected from the group consisting of:
(a) a polypeptide comprising a polypeptide differing from SEQ ID NO: 3 by the lack of at least one of the amino acids at positions 54 (Threonine) to 82 (Glutamic acid) of SEQ ID NO: 3;
(b) a polypeptide comprising a polypeptide differing from SEQ ID NO: 3 by the lack of at least one of the amino acids at positions 54 (Threonine) to 82 (Glutamic acid) and of amino acids 1 to 27 of SEQ ID NO: 3;
(c) a polypeptide comprising SEQ ID NO: 4;
(d) a polypeptide comprising amino acids 28 to 164 of SEQ ID NO: 4;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N51 K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3; and
(g) a polypeptide differing from a), b), c), d), e) or f) by including at least one additional N-linked glycosylation site at position 84, 96, 113, 115, 116 or 141, said position being defined by reference to the position of the amino acid in SEQ ID NO: 3.

47. The isolated polypeptide according to claim 44, wherein said polypeptide is selected from the group consisting of:
(a) a polypeptide consisting of an amino acid sequence differing from SEQ ID NO: 3 by the lack of at least one of the amino acids at positions 54 (Threonine) to 82 (Glutamic acid) of SEQ ID NO: 3;
(b) a polypeptide consisting of an amino acid sequence differing from SEQ ID NO: 3 by the lack of at least one of the amino acids 54 (Threonine) to 82 (Glutamic acid) and of the amino acid 1 to 27 of SEQ ID NO: 3 or;
(c) a polypeptide consisting of SEQ ID NO: 4;
(d) a polypeptide consisting of amino acids 28 to 164 of SEQ ID NO: 4;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, I160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3;
(g) a polypeptide differing from a), b), c), d) or e) by including at least one additional N-linked glycosylation site at position 84, 96, 113, 115, 116 or 141, said position being defined by reference to the position of the amino acid in SEQ ID NO: 3; and
(h) a polypeptide that has at least 80% amino acid sequence identity with the polypeptide of a), b), c), d), e), f) or g).

48. The isolated polypeptide according to claim 44, wherein said polypeptide is selected from the group consisting of:
(a) a polypeptide comprising an amino acid sequence differing from SEQ ID NO: 3 by the lack of at least one of the amino acids at positions 54 (Threonine) to 142 (Glutamine) of SEQ ID NO: 3;
(b) a polypeptide comprising an amino acid sequence differing from SEQ ID NO: 3 by the lack of at least one of the amino acids at positions 54 (Threonine) to 142 (Glutamine) and of the amino acid 1 to 27 of SEQ ID NO: 3;
(c) a polypeptide comprising at SEQ ID NO: 6;
(d) a polypeptide comprising amino acids 28 to 104 of SEQ ID NO: 6;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations chosen in the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, 1160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3; and
(g) a polypeptide differing from a), b), c), d), e) or f) by including at least one additional N-linked glycosylation site at position 84, 96, 113, 115, 116 or 141, said position being defined by reference to the position of the amino acid in SEQ ID NO: 3.

49. The isolated polypeptide according to claim 44, wherein said polypeptide is selected from the group consisting of:
(a) an amino acid sequence differing from SEQ ID NO: 3 by the lack of at least one of the amino acids at positions 54 (Threonine) to 142 (Glutamine) of SEQ ID NO: 3;
(b) a polypeptide differing from SEQ ID NO: 3 by the lack of at least one of the amino acids at positions 54 (Threonine) to 142 (Glutamine) and amino acids 1 to 27 of SEQ ID NO: 3;
(c) a polypeptide consisting of SEQ ID NO: 6;
(d) a polypeptide consisting of amino acids 28 to 104 of SEQ ID NO: 6;
(e) a polypeptide differing from a), b), c) or d) by one, two, three, four, five, six, seven, eight, nine or ten mutations selected from the group consisting of: E40Q, Q85QQ, G104S, L129G, L129P, L129S, S131N, L132F, SL131-132NF, T134D, G140R and S147C, the position of said mutations being defined by reference to the position of the amino acid in SEQ ID NO: 3;
(f) a polypeptide differing from a), b), c) or d) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A, L135S, K143A, S153A, T159A, 1160A, T161A, K167A, F169I, R170A, S173A, N174K, N174A, F175Y, F175A, L176A, R177A, R177E, G178A, K179A, K179W, L180A, K181A, L182A, G185A, C187S, C188A, and R189A, the position of said mutations being defined by reference to the position of the amino acid in SEQ ID NO: 3;
(g) a polypeptide differing from a), b), c), d), e) or f) by including at least one additional N-linked glycosylation site at position 84, 96, 113, 115, 116 or 141, said position being defined by reference to the position of the amino acid in SEQ ID NO: 3; and
(h) a polypeptide having at least 80% amino acid sequence identity with the polypeptide of a), b), c), d), e), f) or g).

50. The isolated polypeptide according to claim 44, wherein said polypeptide is selected from the group consisting of:
(a) SEQ ID NO: 8; and
(b) a polypeptide that is at least 75% identical to SEQ ID NO: 8.

51. The isolated polypeptide according to claim 44, wherein said polypeptide is selected from the group consisting of:
(a) a polypeptide comprising SEQ ID NO: 9;
(b) a polypeptide comprising amino acids 28 to 1 54 of SEQ ID NO: 9;
(c) a polypeptide differing from a) or b) by one, two, three, four, five, six, seven, eight, nine or ten mutations selected from the group consisting of: E40Q, D70N, Q85QQ, G104S, L129G, L129P, L129S, S131N, L132F, T134D, G140R and SL131-132NF;
(d) a polypeptide polypeptide differing from a) or b) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128I, L129A, R130A, S131A, S131I, L132A, T133A, T133I, T134A, T134L, L135K, L135A and L135S;
(e) a polypeptide differing from a), b), c) or d) by including at least one additional N-linked glycosylation site at position 57, 78, 79, 80, 82, 84, 96, 113, 115, 116 or 141; and
(f) a polypeptide having at least 80% amino acid sequence identity with the polypeptide of a), b), c), d) or e).

52. The isolated polypeptide according to claim 50, wherein said polypeptide is selected from the group consisting of:
(a) a polypeptide consisting of SEQ ID NO: 8; and
(b) a polypeptide consisting of a polypeptide having at least 75% amino acid sequence identity with SEQ ID NO: 8.

53. The isolated polypeptide according to claim 51, wherein said polypeptide is selected from the group consisting of:
(a) a polypeptide consisting of SEQ ID NO: 9;
(b) a polypeptide consisting of amino acids 28 to 154 of SEQ ID NO: 9;
(c) a polypeptide differing from a) or b) by one, two, three, four, five, six, seven, eight, nine or ten mutations selected from the group consisting of: E40Q, D70N, Q85QQ, G104S, L129G, L129P, L129S, S131N, L132F, T134D, G140R and SL131-132NF;
(d) a polypeptide differing from a) or b) by one, two, three, four, five, six, seven, eight, nine or ten mutations selected from the group consisting of: I33A, C34S, C34A, R37I, VI38S, L39A, E40A, R41A, R41B, R41E, R41Q, Y42A, Y42F, Y42I, K47A, K47E, E48A, N51K, C56S, C56Y, A57N, H59T, C60S, C60Y, N65K, P69N, P69A, D70A, T71I, K72A, K72D, V73A, N74A, F75A, F75I, Y76A, Y76S, W78F, W78N, K79A, Q86N, E89T, L94S, L97A, N110K, D123R, K124A, S127R, S127E, S127A, S127T, G128A, G128T, L129A, R130A, S131A, S131I, L132A, T133A, T133I, T134A, T134L, L135K, L135A and L135S;
(e) a polypeptide differing from a), b), c) or d) by including at least one additional N-linked glycosylation site at position 57, 78, 79, 80, 82, 84, 96, 1I13, 115, 116 or 141; and
(f) a polypeptide having at least 80% amino acid sequence identity with the polypeptide of a), b), c), d) or e).

54. The isolated polypeptide according to claim 44, wherein said polypeptide: can induce a reduction of the compound muscle action potential (CMAP) latency of at least about 0.02 ms following nerve crush; can stimulate the production of Myelin Basic Protein (MBP) by at least about 5%; retains less than 50% of the hematotrophic activity of wild-type EPO; or increases hematocrit level by less than about 10% compared to the baseline hematocrit level.

55. The isolated polypeptide according to claim 44, further comprising an heterologous moiety selected from the group consisting of cytotoxic agents, labels, drugs, therapeutic agents, natural polymers and synthetic polymers.

56. A fusion protein comprising a polypeptide according to claim 44 operably linked to an additional amino acid domain.

57. The fusion protein according to claim 56, wherein the polypeptide is operably linked to the GST sequence, a His tag sequence, a multimerication domain, the constant region of an immunoglobulin molecule or a heterodimeric protein hormone.

58. An isolated nucleic acid molecule encoding a polypeptide according to claim 44.

59. The isolated nucleic acid molecule according to claim 58, wherein said isolated nucleic acid molecules is a cDNA molecule or a vector.

60. The isolated nucleic acid molecule according to claim 58, wherein said nucleic acid molecules comprise or consist of: SEQ ID NO: 5, SEQ ID NO: 7, SEQ ID NO: 10, SEQ ID NO: 11, SEQ ID NO: 12 or a nucleic acid molecule complementary thereto.

61. A recombinant host cell comprising a nucleic acid molecule according to claim 59.

62. The host cell according to claim 61, wherein said host cell is prokaryotic or eukaryotic.

63. A composition comprising an isolated antibody or antigen binding fragment thereof that selectively binds to a polypeptide according to claim 44.

64. The composition according to claim 63, wherein said antibody or antigen binding fragment thereof further comprises a heterologous moiety.

65. A pharmaceutical composition comprising a polypeptide according to claim 44.

66. A method of producing a polypeptide comprising culturing a recombinant host cell according to claim 61 under conditions allowing expression of the nucleic acid molecule, and recovering the polypeptide produced.

67. A method of treating a disregulation of EPO expression or activity in an individual comprising administering to the patient a pharmaceutical composition of claim 65.

68. A method of treating a disorder in a patient comprising administering to the patient a therapeutically effective amount of a polypeptide according to claim 44, wherein the disorder is selected from the group consisting of: anemia, Chronic Renal Failure patients hypertension, Pediatric patients on dialysis, diseases or conditions associated with insufficient hematocrit levels, disorders connected with chemotherapy treatments, cancers, cardiovascular diseases, and diseases of the central nervous system (CNS) or peripheral nervous system which have primarily neurological or psychiatric symptoms.

69. The method according to claim 68, wherein the disorder is selected from the group consisting of anemia, cancer, Alzheimer's disease, Parkinson's disease, Leigh's disease, amyotrophic lateral sclerosis, multiple sclerosis, ischemia-reperfusion injury, and myocardial infarction.

70. The method according to claim 68, wherein the disorder is an anemia selected from the group consisting of: anemia associated with Chronic Renal Failure (CRF), anemia in Zidovudine-treated HTV-infected patients, anemia in cancer patients on Chemotherapy or radiotherapy, anemia associated with the progression of non-myeloid cancers, anemia associated with viral infection (such as HIV) and anemia of chronic disease or a cancer selected from the group consisting of: adenocarcinoma of the kidney, prostate, ovary or breast, lymphoma, leukaemia, multiple mycloma, tumors affecting the Central Nervous System.

71. A method of treating cancer in a subject comprising administering to the subject an effective amount of an antibody composition according to claim 63.
